(19) United States
(12) Patent Application Publication
(10) Pub. No.: US 2025/0289856 A1
(43) Pub. Date: Sep. 18, 2025

(54) COMPOSITIONS AND METHODS FOR PRODUCING HIGH SECRETED YIELDS OF RECOMBINANT PROTEINS

(71) Applicant: BOLT THREADS, INC., Emeryville, CA (US)

(72) Inventors: Joshua Kittleson, Berkeley, CA (US); Thomas Stevens, Emeryville, CA (US); Rena Hill, Emeryville, CA (US); Carlos Gustavo Pesce, Emeryville, CA (US); David N. Breslauer, San Francisco, CA (US); Daniel M. Widmaier, San Francisco, CA (US)

(21) Appl. No.: 19/223,644

(22) Filed: May 30, 2025

Related U.S. Application Data
(63) Continuation of application No. 18/341,669, filed on Jun. 26, 2023, now Pat. No. 12,325,730.
Application No. 18/341,669 is a continuation of application No. 17/695,219, filed on Mar. 15, 2022, now Pat. No. 11,725,030.
Application No. 17/695,219 is a continuation of application No. 15/920,291, filed on Mar. 13, 2018, now Pat. No. 11,306,127.
Application No. 15/920,291 is a continuation of application No. PCT/US18/21812, filed on Mar. 9, 2018.
(60) Provisional application No. 62/470,144, filed on Mar. 10, 2017.

(51) Int. Cl. C07K 14/435; C07K 14/395; C12P 21/02

(52) U.S. Cl. CPC C07K 14/43586; C07K 14/395; C12P 21/02; C07K 2319/02

(57) ABSTRACT

The present disclosure relates to methods for producing recombinant proteins, as well as compositions used in and produced by such methods. Specifically, the present disclosure relates to methods for producing high secreted yields of recombinant proteins, and the compositions provided herein include expression constructs, recombinant vectors, and recombinant host cells that comprise polynucleotide sequences encoding proteins operably linked to recombinant secretion signals that comprise the leader peptide of the α-mating factor (αMF) of Saccharomyces cerevisiae and a non-αMF signal peptide.

### CROSS-REFERENCE TO RELATED APPLICATIONS

This application is a continuation of U.S. patent application Ser. No. 18/341,669, filed on Jun. 26, 2023, which is a continuation of U.S. patent application Ser. No. 17/695,219, filed on Mar. 15, 2022, which is a continuation of U.S. patent application Ser. No. 15/920,291, filed on Mar. 13, 2018, which is a bypass continuation of International Application No. PCT/US2018/021812, filed Mar. 9, 2018, which claims the benefit of U.S. Provisional Patent Application No. 62/470,144, filed Mar. 10, 2017, the disclosures of which are incorporated herein by reference in their entirety.

### SEQUENCE LISTING

The instant application contains a Sequence Listing which has been submitted electronically in xml format and is hereby incorporated by reference in its entirety. Said listing, created on May 23, 2025, is named “50008D_SeqListing.xml” and is 268,711 bytes in size.

### FIELD OF THE INVENTION

The present disclosure relates to methods for producing recombinant proteins, as well as compositions used in and produced by such methods. Specifically, the present disclosure relates to methods for producing high secreted yields of recombinant proteins, as well as expression constructs, recombinant vectors, recombinant host cells, and fermentations used in such methods.

### BACKGROUND OF THE INVENTION

Many proteins needed for research, industrial, or therapeutic purposes (e.g., enzymes, vaccines, hormones, and biopharmaceutical proteins) are produced industrially in recombinant host cells. Yeasts, in particular budding yeasts, are favored eukaryotic host organisms for such application. Yeast cells grow rapidly to high cell density in inexpensive media, and comprise cellular machinery for protein folding and post-translational modification (e.g., proteolytic maturation, disulfide bond formation, phosphorylation, O- and N-linked glycosylation). The most commonly used yeast species for production of recombinant proteins include Saccharomyces cerevisiae, Pichia pastoris, Hansenula polymorpha, and Kluyveromyces lactis. Of these, Pichia pastoris is particularly suitable for applications in which recombinant proteins are to be produced at larger (e.g., industrial) scale because it can achieve high density cell growth.

Industrial scale production of recombinant proteins in recombinant host cells is facilitated when the recombinant proteins are secreted from the cells because secreted proteins are readily separated from intact cells, obviating the need for cellular lysis and subsequent separation of the proteins from cellular debris. Pichia pastoris is particularly suitable for production of secreted recombinant proteins because it can grow in minimal salt media, which permits isolation of secreted proteins via filtration and chromatography at low conductivity, and because Pichia pastoris natively secretes relatively few fermentative products (i.e., small proteins), which further facilitates isolation and purification of secreted recombinant proteins.

Recombinant host cells used for production of secreted recombinant proteins ideally produce large quantities of the recombinant protein, and secrete large fractions of the recombinant protein produced. The former is typically achieved by employing strategies well known in the art, such as, for example, codon optimizing the polynucleotide sequences that are engineered into the recombinant host cells and that encode the recombinant proteins, placing the transcription of such polynucleotide sequences under the control of strong promoters and effective terminators, optimizing translation by introducing suitable ribose binding sites, and increasing the copy number of the polynucleotide sequences in the recombinant host cells (e.g., by engineering host cells that comprise 2 or more copies of a particular polynucleotide sequence). These strategies, however, tend to reach a natural limit in their effectiveness as high copy numbers genetically destabilize the recombinant host cells, and strong promoters yield higher levels of the recombinant proteins than the recombinant host cells can properly fold and/or secrete (Damasceno et al. Appl Microbiol Biotechnol 93:31-39; Parekh et al.

Protein Expr Purif. 6 (4): 537-45; Zhu et al. J Appl Microbiol 107:954-963; Liu et al.

Protein Expr. Purif. 30:262-274). As a result, yields of the recombinant proteins tend to plateau or even decline as unfolded or mis-folded recombinant proteins accumulate inside the recombinant host cells and the recombinant host cells activate molecular stress responses (e.g., the unfolded protein response [UPR] or the ER-associated protein degradation pathway [ERAD] (Hohenblum et al. Biotechnol Bioeng. 12:367-375; Vassileva et al. J Biotechnol. 12:21-35; Inan et al. Biotechnol Bioeng. 12:771-778; Zhu et al. J Appl Microbiol. 12 (3): 954-963). Indeed, up-regulation of chaperone proteins or of the main UPR transcriptional regulator (Hac1p) have been shown to reduce the effects of the UPR and to boost recombinant protein yields (Zhang et al. Biotechnol Prog. 12:1090-1095; Lee et al. Process Biochem. 12:2300-2305; Valkonen et al. Appl Environ Microbiol. 12:6979-6986). However, such measures have produced mixed results (Guerfal et al. Microb Cell Fact. 12:49) and still do not completely eliminate the saturation of the secretory pathways of recombinant host cells (Inan et al. Biotechnol Bioeng. 12:771-778). The capacity of the secretion machinery of recombinant host cells thus remains a major bottleneck for production of recombinant proteins.

What is needed therefore, are methods and compositions that allow increased expression of desirable recombinant proteins while alleviating the negative impact of overexpression on the recombinant host cells.

### BRIEF DESCRIPTION OF THE DRAWINGS

FIG. 1 is a flow diagram of methods for producing high secreted yields of recombinant proteins.

FIG. 2 is an illustrative map of a recombinant vector that comprises a polynucleotide sequence that encodes a silk-like protein operably linked to an N-terminal recombinant secretion signal comprising a functional variant of the leader peptide of the α-mating factor of Saccharomyces cerevisiae (*pro-αMF (sc)) and a signal peptide. The amino acid sequences for the various signal peptides and recombinant secretion signals used are given in Tables A and B.

FIGS. 3 through 6 show extracellular (secreted) yields of the recombinant silk-like protein produced by various Pichia pastoris recombinant host cells as assayed by ELISA.

FIG. 7 is a diagram of a recombinant vector comprising an expression construct for expressing a polypeptide with recombinant secretion signals, according to an embodiment of the invention.

FIG. 8 illustrates secretion levels of alpha-amylase from Pichia pastoris transformed to express recombinant alpha-amylase with various recombinant secretion signals.

FIG. 9 illustrates secretion levels of fluorescent protein from Pichia pastoris transformed to express recombinant fluorescent protein with various recombinant secretion signals.

The figures depict various embodiments of the present disclosure for purposes of illustration only. One skilled in the art will readily recognize from the following discussion that alternative embodiments of the structures and methods illustrated herein may be employed without departing from the principles described herein.

### DETAILED DESCRIPTION OF THE INVENTION

### Definitions

Unless defined otherwise, all technical and scientific terms used herein have the same meaning as is commonly understood by one of ordinary skill in the art to which this disclosure pertains

The terms “a” and “an” and “the” and similar referents as used herein refer to both the singular and the plural, unless otherwise indicated herein or clearly contradicted by context.

Amino acids can be referred to by their single-letter codes or by their three-letter codes. The single-letter codes, amino acid names, and three-letter codes are as follows: G-Glycine (Gly), P-Proline (Pro), A-Alanine (Ala), V-Valine (Val), L-Leucine (Leu), I-Isoleucine (IIe), M-Methionine (Met), C-Cysteine (Cys), F-Phenylalanine (Phe), Y-Tyrosine (Tyr), W-Tryptophan (Trp), H-Histidine (His), K-Lysine (Lys), R-Arginine (Arg), Q-Glutamine (Gln), N Asparagine (Asn), E-Glutamic Acid (Glu), D-Aspartic Acid (Asp), S-Serine (Ser), T-Threonine (Thr).

The term “functional variant” as used herein refers to a protein that differs in composition from a native protein, where the functional properties are preserved to within 10% of the native protein properties. In some embodiments, the difference between the functional variant and the native protein can be in primary amino acid sequence (e.g., one or more amino acids are removed, inserted, or substituted) or post-translation modifications (e.g., glycosylation, phosphorylation). Amino acid insertions may comprise N-terminal and/or C-terminal fusions as well as intra-sequence insertions of single or multiple amino acids. Amino acid substitution includes non-conservative and conservative substitutions, where conservative amino acid substitution tables are well known in the art (see, for example, Creighton (1984) Proteins. W. H. Freeman and Company (Eds)). In some embodiments, the functional variant and the native protein have an at least 80%, at least 85%, at least 90%, at least 95%, or at least 99% amino acid or nucleotide sequence identity.

The terms “identity” or “identical” in the context of nucleic acid or amino acid sequences as used herein refer to the nucleotide or amino acid residues in the two sequences that are the same when the sequences are aligned for maximum correspondence. Depending on the application, the percent “identity” can exist over a region of the sequences being compared (i.e., subsequence [e.g., over a functional domain]) or, alternatively, exist over the full length of the sequences. A “region” is considered to be a continuous stretch of at least 9, 20, 24, 28, 32, or 36 nucleotides, or at least 6 amino acids. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm (see, for example, Altschul et al. J. Mol. Biol. 215:403-410; Gish & States. Nature Genet. 3:266-272; Madden et al. Meth. Enzymol. 266:131-141; Altschul et al. Nucleic Acids Res. 25:3389-3402; Zhang 7 Madden. Genome Res. 7:649-656). Software for performing BLAST analyses is publicly available through the National Center for Biotechnology Information. Such software also can be used to determine the mole percentage of any specified amino acid found within a polypeptide sequence or within a domain of such a sequence. As the person of ordinary skill will recognize such percentages also can be determined through inspection and manual calculation.

The terms “including,” “includes,” “having,” “has,” “with,” or variants thereof are intended to be inclusive in a manner similar to the term “comprising”.

The term “microbe” as used herein refers to a microorganism, and refers to a unicellular organism. As used herein, the term includes all bacteria, all archaea, unicellular protista, unicellular animals, unicellular plants, unicellular fungi, unicellular algae, all protozoa, and all chromista.

The term “native” as used herein refers to what is found in nature in its natural, unmodified state.

The term “operably linked” as used herein refers to polynucleotide or amino acid sequences that are in contiguous linkage with a polynucleotide sequence encoding a protein or a protein, as well as to polynucleotide or amino acid sequences that act in trans or at a distance to a polynucleotide sequence encoding a protein, and that control the transcription, translation, folding, secretion, or other functional aspect of the polynucleotide encoding the protein or the protein.

The terms “optional” or “optionally” mean that the feature or structure may or may not be present, or that an event or circumstance may or may not occur, and that the description includes instances where a particular feature or structure is present and instances where the feature or structure is absent, or instances where the event or circumstance occurs and instances where the event or circumstance does not occur.

The term “protein” as used herein refers to both a polypeptide without functional structure and a polypeptide that folds into an active structure.

The term “recombinant protein” as used herein refers to a protein that is produced in a recombinant host cell, or to a protein that is synthesized from a recombinant nucleic acid.

The term “recombinant host cell” as used herein refers to a host cell that comprises a recombinant nucleic acid.

The term “recombinant nucleic acid” as used herein refers to a nucleic acid that is removed from its naturally occurring environment, or a nucleic acid that is not associated with all or a portion of a nucleic acid abutting or proximal to the nucleic acid when it is found in nature, or a nucleic acid that is operatively linked to a nucleic acid that it is not linked to in nature, or a nucleic acid that does not occur in nature, or a nucleic acid that contains a modification that is not found in that nucleic acid in nature (e.g., insertion, deletion, or point mutation introduced artificially, e.g., by human intervention), or a nucleic acid that is integrated into a chromosome at a heterologous site. The term includes cloned DNA isolates and nucleic acids that comprise chemically-synthesized nucleotide analog.

The term “recombinant secretion signal” as used herein refers to a secretion signal that comprises a non-native combination of a signal peptide and a leader peptide.

The term “recombinant vector” as used herein refers to a nucleic acid molecule capable of transporting another nucleic acid molecule to which it has been linked. The term includes “plasmids”, which generally refers to a circular double stranded DNA loop into which additional DNA segments can be ligated, and linear double-stranded molecules, such as those resulting from amplification by the polymerase chain reaction (PCR) or from treatment of a plasmid with a restriction enzyme. Other non-limiting examples of vectors include bacteriophages, cosmids, bacterial artificial chromosomes (BAC), yeast artificial chromosomes (YAC), and viral vectors (i.e., complete or partial viral genomes into which additional DNA segments are ligated). Certain vectors are capable of autonomous replication in a recombinant host cell into which they are introduced (e.g., vectors having an origin of replication that functions in the cell). Other vectors upon introduction can be integrated into the genome of a recombinant host cell, and are thereby replicated along with the cell genome.

The term “secreted recombinant protein” as used herein refers to a recombinant protein that is exported across the cellular membrane and/or cell wall of a recombinant host cell that produces the recombinant protein.

The term “secreted yield” as used herein refers to the amount of secreted protein produced by a host cell based on a fixed amount of carbon supplied to a fermentation comprising the host cell.

The term “total yield” as used herein refers to the amount of total protein produced by a host cell based on a fixed amount of carbon supplied to a fermentation comprising the host cell.

The term “truncated” as used herein refers to a protein sequence that is shorter in length than a native protein. In some embodiments, the truncated protein can be greater than 10%, or greater than 20%, or greater than 30%, or greater than 40%, or greater than 50%, or greater than 60%, or greater than 70%, or greater than 80%, or greater than 90% of the length of the native protein.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Wherever a range of values is recited, that range includes every value falling within the range, as if written out explicitly, and further includes the values bounding the range. Thus, a range of “from X to Y” includes every value falling between X and Y, and includes X and Y.

### Compositions and Methods for Producing High Secreted Yields of Recombinant Proteins

Provided herein are expression constructs, recombinant vectors, recombinant host cells, and fermentations, and methods that use such expression constructs, recombinant vectors, recombinant host cells, and fermentations for producing high secreted yields of recombinant proteins.

Advantages of the compositions and methods provided herein include that they provide cost-effective means for producing large quantities of recombinant proteins. The large quantities are obtained using recombinant host cells that secrete recombinant proteins via their secretory pathways. Such secretion of recombinant proteins a) avoids toxicity from intracellular accumulation of recombinant proteins; b) simplifies purification by eliminating cell disruption, separation from cellular components, and protein refolding processes; and c) provides properly folded recombinant proteins with post-translational modifications that may be critical to the activity/function of the recombinant proteins.

### Expression Constructs

Provided herein are expression constructs comprising polynucleotide sequences that encode proteins operably linked to recombinant secretion signals provided herein. The recombinant secretion signals are typically operably linked to the N-termini of the proteins.

### Recombinant Secretion Signals

To be secreted, a protein has to travel through the intracellular secretory pathway of a cell that produces it. The protein is directed to this pathway, rather than to alternative cellular destinations, via an N-terminal secretion signal. At a minimum, a secretion signal comprises a signal peptide. Signal peptides typically consist of 13 to 36 mostly hydrophobic amino acids flanked by N-terminal basic amino acids and C-terminal polar amino acids. The signal peptide interacts with the signal recognition particle (SRP) or other transport proteins (e.g., SND, GET) that mediates the co- or post-translational translocation of the nascent protein from the cytosol into the lumen of the ER. In the ER, the signal peptide is typically cleaved off and the protein folds and undergoes post-translational modifications. The protein is then delivered from the ER to the Golgi apparatus and then on to secretory vesicles and the cell exterior. In addition to a signal peptide, a subset of nascent proteins natively destined for secretion carry a secretion signal that also comprises a leader peptide. Leader peptides typically consist of hydrophobic amino acids interrupted by charged or polar amino acids. Without wishing to be bound by theory, it is believed that the leader peptide slows down transport and ensures proper folding of the protein, and/or facilitates transport of the protein from the ER to the Golgi apparatus, where the leader peptide is typically cleaved off.

The amount of protein that is secreted from a cell varies significantly between proteins, and is dependent in part on the secretion signal that is operably linked to the protein in its nascent state. A number of secretion signals are known in the art, and some are commonly used for production of secreted recombinant proteins. Prominent among these is the secretion signal of the a-mating factor (αMF) of Saccharomyces cerevisiae, which consists of a N-terminal 19-amino-acid signal peptide (also referred to herein as pre-αMF (sc)) followed by a 70-amino-acid leader peptide (also referred to herein as pro-αMF (sc); SEQ ID NO: 1). Inclusion of pro-αMF (sc) in the secretion signal of the αMF of Saccharomyces cerevisiae (also referred to herein as pre-αMF (sc)/pro-αMF (sc) (SEQ ID NO: 115) has proven critical for achieving high secreted yields of proteins (see, for example, Fitzgerald & Glick Microb Cell Fact 28; 13 (1): 125; Fahnestock et al. J Biotechnol 74 (2): 105). Addition of pro-αMF (sc) or functional variants thereof to signal peptides other than pre-αMF (sc) has also been explored as a means of achieving secretion of recombinant proteins, but has shown variable degrees of effectiveness, increasing secretion for certain recombinant proteins in certain recombinant host cells but having no effect or decreasing secretion for other recombinant proteins (Fitzgerald & Glick. Microb Cell Fact 28; 13 (1): 125; Liu et al. Biochem Biophys Res Commun. 326 (4): 817-24; Obst et al. ACS Synth Biol. 2017 Mar. 2).

The invention provided herein is based on the identification by the inventors of recombinant secretion signals comprising a functional variant of native pro-αMF (sc) (referred to herein as *pro-αMF (sc)) in combination with certain signal peptides other than pre-αMF (sc) that provide for various secreted yields of recombinant proteins. In some embodiments, the recombinant secretion signals provide larger secreted yields of recombinant proteins than is achieved with the secretion signal of the a-mating factor (αMF) of Saccharomyces cerevisiae and/or the recombinant secretion signals in the prior art (e.g., pre-OST1 (sc)/pro-αMF (sc); see Fitzgerald & Glick. Microb Cell Fact 28; 13 (1): 125; Liu et al. Biochem Biophys Res Commun. 326 (4): 817-24; Obst et al. ACS Synth Biol. 2017 Mar. 2). In other embodiments, the recombinant secretion signals provide smaller secreted yields of recombinant proteins than is achieved with the secretion signal of the a-mating factor (αMF) of Saccharomyces cerevisiae.

Accordingly, in various embodiments, the expression constructs provided herein comprise polynucleotide sequences that encode proteins operably linked to recombinant secretion signals that comprise a leader peptide and a signal peptide, wherein the leader peptide is pro-αMF (sc) (SEQ ID NO: 1) or a functional variant thereof than has an at least 80% amino acid sequence identity to SEQ ID NO: 1, and wherein the signal peptide does not comprise pre-αMF (sc).

In some embodiments, the functional variant is native pro-αMF (sc) comprising one or two substituted amino acids. In some embodiments, the functional variant is *pro-αMF (SEQ ID NO: 2). In some embodiments, the functional variant has an at least 85%, at least 90%, at least 95%, or at least 99% amino acid sequence identity to SEQ ID NO: 1. In some embodiments, the functional variant is αMF_no_EAEA or αMFΔ or αMFΔ_no_Kex (Obst et al. ACS Synth Biol. 2017 Mar. 2).

In some embodiments, the signal peptide selected from Table 1 or is a functional variant that has an at least 80% amino acid sequence identity to a signal peptide selected from Table 1. In some embodiments, the functional variant is a signal peptide selected from Table 1 that comprises one or two substituted amino acids. In some such embodiments, the functional variant has an at least 85%, at least 90%, at least 95%, or at least 99% amino acid sequence identity to a signal peptide selected from Table 1. In some embodiments, the signal peptide mediates translocation of the nascent recombinant protein into the ER post-translationally (i.e., protein synthesis precedes translocation such that the nascent recombinant protein is present in the cell cytosol prior to translocating into the ER). In other embodiments, the signal peptide mediates translocation of the nascent recombinant protein into the ER co-translationally (i.e., protein synthesis and translocation into the ER occur simultaneously). An advantage of using a signal peptide that mediates co-translational translocation into the ER is that recombinant proteins prone to rapid folding are prevented from assuming conformations that hinder translocation into the ER and thus secretion.

TABLE 1
Signal Peptides

| Source Gene ID | Species | Name | SEQ ID | Amino Acid Sequence |
|---|---|---|---|---|
| P01239 | Bos taurus | pre-PRL(bt) | 3 | MDSKGSSQKGSRLLLLLVVSNLLLCSA |
| P00698 | Gallus gallus | pre-CLSP(gg) | 4 | MRSLLILVLCFLPLAALG |
| PRC1 | Saccharomyces | pre-CPY(sc) | 5 | MKAFTSLLCGLGLSTTLAKA |
| | cerivisae | | | |
| PAS_chr2-2_0084 | Pichia pastoris | pre- | 6 | MDSEPLLPNPNDSRKPANWRRIIKYISLTLAWIGIFSYVYIYHGTA |
| | | PHO8(pp) | | |
| PEP4 | Saccharomyces | pre-PEP4(sc) | 7 | MFSLKALLPLALLLVSANQVAA |
| | cerevisiae | | | |
| SUC2 | Saccharomyces | pre-SUC2(sc) | 8 | MLLQAFLFLLAGFAAKISA |
| | cerevisiae | | | |
| EPX1 | Pichia pastoris | pre-EPX1(pp) | 9 | MKLSTNLILAIAAASAVVSA |
| DAP2 | Saccharomyces | pre-DAP2(sc) | 116 | MEGGEEEVERIPDELFDTKKKHLLDKLIRVGIILVLLIWGTVLLLKSI |
| | cerivisae | | | |
| KAR2 | Saccharomyces | pre- | 117 | MFFNRLSAGKLLVPLSVVLYALFVVILPLQNSFHSSNVLVRGA |
| | cerivisae | KAR2(sc) | | |
| NCP1 | Saccharomyces | pre-NCP1(sc) | 118 | MPFGIDNTDFTVLAGLVLAVLLYVKR |
| | cerivisae | | | |
| RRT12 | Saccharomyces | pre- | 119 | MKPQCILISLLVNLAYA |
| | cerivisae | RRT12(sc) | | |
| PGU1 | Saccharomyces | pre-PGU1(sc) | 120 | MISANSLLISTLCAFAIATPLSKR |
| | cerivisae | | | |
| SRL1 | Saccharomyces | pre-SRL1(sc) | 121 | MLQSVVFFALLTFASSVSA |
| | cerivisae | | | |
| P08721 | Rattus | pre-OSP(m) | 122 | MRLAVVCLCLFGLASCLPVKV |
| | norvegicus | | | |
| PAS_chr2-1_0140 | Pichia pastoris | pre- | 123 | MLSLKPSWLTLAALMYAMLLVVVPFAKPVRA |
| | | KAR2(pp) | | |
| PAS_chr1-1_0130 | Pichia pastoris | pre-DSE4(pp) | 124 | MSFSSNVPQLFLLLVLLTNIVSG |
| PAS_chr2-1_0454 | Pichia pastoris | pre- | 125 | MNLYLITLLFASLCSA |
| | | EXG1(pp) | | |
| GET1 | Saccharomyces | pre-GET1(sc) | 126 | MHWAAAVAIFFIVVTKFLQ |
| | cerivisae | | | |
| PAS_chr1-3_0229 | Pichia pastoris | pre- | 127 | MRFSNFLTVSALLTGALG |
| | | SCW10(pp) | | |
| CTS1 | Saccharomyces | pre-CTS1(sc) | 128 | MSLLYIILLFTQFLLLPTDA |
| | cerivisae | | | |
| PAS_chr1-3_0251 | Pichia pastoris | pre- | 129 | MAKADGSLLYYNPHNPPRRYYFYMAIFAVSVICVLYGPSQQLSS |
| | | OCH1(pp) | | |

Thus, in some embodiments, the expression constructs comprise polynucleotide sequences that encode proteins operably linked to recombinant secretion signal that are selected from Table 2 or are functional variants that have an at least 80% amino acid sequence identity to a recombinant secretions signal selected from Table 2. In some such embodiments, the functional variant has an at least 85%, at least 90%, at least 95%, or are at least 99% amino acid sequence identity to a recombinant secretion signal selected from Table 2.

In some embodiments, the expression constructs provided herein comprise the polynucleotide sequences in multiple (e.g., 2, 3, 4, 5, etc.) copies. In some such embodiments, the polynucleotide sequences are identical. In other such embodiments, at least 2 of the polynucleotide sequences are not identical. In embodiments in which at least 2 of the polynucleotide sequences are not identical, the at least 2 polynucleotide sequences may differ from each other in the proteins and/or the recombinant secretion signals and/or optional tag peptides or polypeptides (see below) they encode.

TABLE 2
Recombinant Secretion Signals

| Name | SEQ ID NO | Amino Acid Sequence |
|---|---|---|
| pre-PRL(bt)/ | 10 | MDSKGSSQKGSRLLLLLVVSNLLLCSAAPVNTTTEDETAQIPAEAVIGYSDLEGDFDVAVLP |
| *pro-αMF(sc) | | FSNSTNNGLLFINTTIASIAAKEEGVSLEKREAEA |
| pre-CLSP(gg)/ | 11 | MRSLLILVLCFLPLAALGAPVNTTTEDETAQIPAEAVIGYSDLEGDFDVAVLPFSNSTNNGLL |
| *pro-αMF(sc) | | FINTTIASIAAKEEGVSLEKREAEA |
| pre-CPY(sc)/ | 12 | MKAFTSLLCGLGLSTTLAKAAPVNTTTEDETAQIPAEAVIGYSDLEGDFDVAVLPFSNSTNN |
| *pro-αMF(sc) | | GLLFINTTIASIAAKEEGVSLEKREAEA |
| pre-PH08(pp)/ | 13 | MDSEPLLPNPNDSRKPANWRRIIKYISLTLAWIGIFSYVYIYHGTAAPVNTTTEDETAQIPAEA |
| *pro-αMF(sc) | | VIGYSDLEGDFDVAVLPFSNSTNNGLLFINTTIASIAAKEEGVSLEKREAEA |
| pre-PEP4(sc)/ | 14 | MFSLKALLPLALLLVSANQVAAAPVNTTTEDETAQIPAEAVIGYSDLEGDFDVAVLPFSNST |
| *pro-αMF(sc) | | NNGLLFINTTIASIAAKEEGVSLEKREAEA |
| pre-SUC2(sc)/ | 15 | MLLQAFLFLLAGFAAKISAAPVNTTTEDETAQIPAEAVIGYSDLEGDFDVAVLPFSNSTNNGL |
| *pro-αMF(sc) | | LFINTTIASIAAKEEGVSLEKREAEA |
| pre-OCH1(pp)/ | 16 | MAKADGSLLYYNPHNPPRRYYFYMAIFAVSVICVLYGPSQQLSSAPVNTTTEDETAQIPAEA |
| *pro-αMF(sc) | | VIGYSDLEGDFDVAVLPFSNSTNNGLLFINTTIASIAAKEEGVSLEKREAEA |
| pre-EPX1(pp)/ | 130 | MKLSTNLILAIAAASAVVSAAPVNTTTEDETAQIPAEAVIGYSDLEGDFDVAVLPFSNSTNNG |
| *pro-αMF(sc) | | LLFINTTIASIAAKEEGVSLEKREAEA |
| pre-DAP2(sc)/ | 131 | MEGGEEEVERIPDELFDTKKKHLLDKLIRVGIILVLLIWGTVLLLKSIAPVNTTTEDETAQIPA |
| *pro-αMF(sc) | | EAVIGYSDLEGDFDVAVLPFSNSTNNGLLFINTTIASIAAKEEGVSLEKREAEA |
| pre-KAR2(sc)/ | 132 | MFFNRLSAGKLLVPLSVVLYALFVVILPLQNSFHSSNVLVRGAAPVNTTTEDETAQIPAEAVI |
| *pro-αMF(sc) | | GYSDLEGDFDVAVLPFSNSTNNGLLFINTTIASIAAKEEGVSLEKREAEA |
| pre-NCP1(sc)/ | 133 | MPFGIDNTDFTVLAGLVLAVLLYVKRAPVNTTTEDETAQIPAEAVIGYSDLEGDFDVAVLPF |
| *pro-αMF(sc) | | SNSTNNGLLFINTTIASIAAKEEGVSLEKREAEA |
| pre-RRT12(sc)/ | 134 | MKPQCILISLLVNLAYAAPVNTTTEDETAQIPAEAVIGYSDLEGDFDVAVLPFSNSTNNGLLF |
| *pro-αMF(sc) | | INTTIASIAAKEEGVSLEKREAEA |
| pre-PGUl(sc)/ | 135 | MISANSLLISTLCAFAIATPLSKRAPVNTTTEDETAQIPAEAVIGYSDLEGDFDVAVLPFSNST |
| *pro-αMF(sc) | | NNGLLFINTTIASIAAKEEGVSLEKREAEA |
| pre-SRL1(sc)/ | 136 | MLQSVVFFALLTFASSVSAAPVNTTTEDETAQIPAEAVIGYSDLEGDFDVAVLPFSNSTNNGL |
| *pro-αMF(sc) | | LFINTTIASIAAKEEGVSLEKREAEA |
| pre-OSP(m)/ | 137 | MRLAVVCLCLFGLASCLPVKVAPVNTTTEDETAQIPAEAVIGYSDLEGDFDVAVLPFSNSTN |
| *pro-αMF(sc) | | NGLLFINTTIASIAAKEEGVSLEKREAEA |
| pre-KAR2(pp)/ | 138 | MLSLKPSWLTLAALMYAMLLVVVPFAKPVRAAPVNTTTEDETAQIPAEAVIGYSDLEGDFD |
| *pro-αMF(sc) | | VAVLPFSNSTNNGLLFINTTIASIAAKEEGVSLEKREAEA |
| pre-DSE4(pp)/ | 139 | MSFSSNVPQLFLLLVLLTNIVSGAPVNTTTEDETAQIPAEAVIGYSDLEGDFDVAVLPFSNST |
| *pro-αMF(sc) | | NNGLLFINTTIASIAAKEEGVSLEKREAEA |
| pre-EXG1(pp)/ | 140 | MNLYLITLLFASLCSAAPVNTTTEDETAQIPAEAVIGYSDLEGDFDVAVLPFSNSTNNGLLFI |
| *pro-αMF(sc) | | NTTIASIAAKEEGVSLEKREAEA |
| pre-GET1(sc)/ | 141 | MHWAAAVAIFFIVVTKFLQAPVNTTTEDETAQIPAEAVIGYSDLEGDFDVAVLPFSNSTNNG |
| *pro-αMF(sc) | | LLFINTTIASIAAKEEGVSLEKREAEA |
| pre-SCW10(pp)/ | 142 | MRFSNFLTVSALLTGALGAPVNTTTEDETAQIPAEAVIGYSDLEGDFDVAVLPFSNSTNNGL |
| *pro-αMF(sc) | | LFINTTIASIAAKEEGVSLEKREAEA |
| pre-CTS1(sc)/ | 143 | MSLLYIILLFTQFLLLPTDAAPVNTTTEDETAQIPAEAVIGYSDLEGDFDVAVLPFSNSTNNGL |
| *pro-αMF(sc) | | LFINTTIASIAAKEEGVSLEKREAEA |

### Recombinant Proteins

The proteins encoded by the polynucleotide sequences comprised in the expression constructs provided herein may be any protein.

In some embodiments, the proteins are silk or silk-like proteins. Such silk or silk-like proteins can be selected from a vast array of full-length or truncated native silk proteins or of functional variants of full-length or truncated native silk proteins, or comprise domains of native silk proteins or of functional variants of silk proteins. Putative native silk proteins can be identified by searching sequence databases (e.g., GenBank) for relevant terms (e.g., silkworm silk, spider silk, spidroin, fibroin, MaSp), and translating any nucleotide sequences into amino acid sequences.

In some embodiments, the silk or silk or silk-like proteins are full-length or truncated native silk proteins of a silkworm, or functional variants of full-length or truncated native silk proteins of a silkworm, or comprise domains of native or functional variants of native silk proteins of a silkworm. In some such embodiments, the silkworm is Bombyx mori.

In some embodiments, the silk or silk or silk-like proteins are full-length or truncated native silk proteins of a spider, or functional variants of full-length or truncated native silk proteins of a spider, or comprise domains of native or functional variants of native silk proteins of a spider. In some embodiments, the native silk proteins are selected from the group consisting of Major Ampullate spider fibroin (MaSp, also called dragline; e.g., MaSp1, MaSp2) silk proteins, Minor Ampullate spider fibroin (MiSp) silk proteins, Flagelliform spider fibroin (Flag) silk proteins, Aciniform spider fibroin (AcSp) silk proteins, Tubuliform spider fibroin (TuSp) silk proteins, and Pyriform spider fibroin (PySp) silk proteins of orb weaving spiders. In some embodiments, the spider is selected from the group consisting of Agelenopsis aperta, Aliatypus gulosus, Aphonopelma seemanni, Aptostichus sp. AS21 7, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

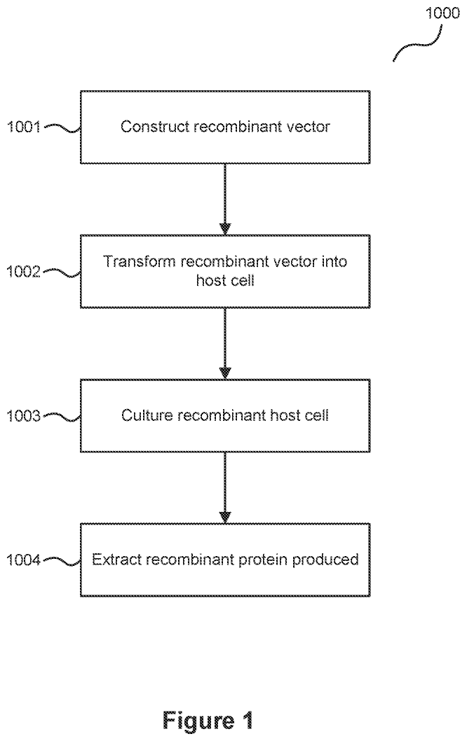

Typically, silk proteins are large proteins (>150 kDa, >1000 amino acids) that can be broken down into 3 domains: an N-terminal non-repetitive domain (NTD), a repeat domain (REP), and a C-terminal non-repetitive domain (CTD). The REP comprises blocks of amino acid sequences (“repeat units”) that are at least 12 amino acids long and that are repeated either perfectly (“exact-repeat units”) or imperfectly (“quasi-repeat units”), and that can comprise 2 to 10 amino acid long sequence motifs (see FIG. 1). REPs typically make up about 90% of the native spider silk proteins, and assemble into the alanine-rich nano-crystalline (<10 nm) domains (likely made up of alternating beta sheets) and glycine-rich amorphous domains (possibly containing alpha-helices and/or beta-turns) that, without wanting to be bound by theory, are believed to confer strength and flexibility to spider silk fibers, respectively. The lengths and compositions of the REPs are known to vary among different spider silk proteins and across different spider species, giving rise to a broad range of silk fibers with specific properties.

In some embodiments, the silk or silk-like proteins comprise one or more native or functional variants of native REPs (e.g., 1, 2, 3, 4, 5, 6, 7, 8), zero or more native or functional variants of NTDs (e.g., 0, 1), and zero or more native or functional variants of native CTDs (e.g., 0, 1). In some embodiments, the silk or silk-like proteins comprise one or more NTDs that each comprise from 75 to 350 amino acids. In some embodiments, the silk or silk or silk-like proteins comprise one or more CTDs that each comprise from 75 to 350 amino acids. In some embodiments, the silk or silk or silk-like proteins comprise one or more REPs that comprise repeat units that each comprise more than 60, more than 100, more than 150, more than 200, more than 250, more than 300, more than 350, more than 400, more than 450, more than 500, more than 600, more than 700, more than 800, more than 900, more than 1000, more than 1250, more than 1500, more than 1750, or more than 2000; from 60 to 2000, to 1750, to 1500, to 1250, to 1000, to 900, to 800, to 700, to 600, to 500, to 450, to 400, to 350, to 300, to 250, to 200, to 150, or to 100; from 100 to 2000, to 1750, to 1500, to 1250, to 1000, to 900, to 800, to 700, to 600, to 500, to 450, to 400, to 350, to 300, to 250, to 200, or to 150; from 150 to 2000, to 1750, to 1500, to 1250, to 1000, to 900, to 800, to 700, to 600, to 500, to 450, to 400, to 350, to 300, to 250, or to 200; from 200 to 2000, to 1750, to 1500, to 1250, to 1000, to 900, to 800, to 700, to 600, to 500, to 450, to 400, to 350, to 300, or to 250; from 250 to 2000, to 1750, to 1500, to 1250, to 1000, to 900, to 800, to 700, to 600, to 500, to 450, to 400, to 350, or to 300; from 300 to 2000, to 1750, to 1500, to 1250, to 1000, to 900, to 800, to 700, to 600, to 500, to 450, to 400, or to 350; from 350 to 2000, to 1750, to 1500, to 1250, to 1000, to 900, to 800, to 700, to 600, to 500, to 450, or to 400; from 400 to 2000, to 1750, to 1500, to 1250, to 1000, to 900, to 800, to 700, to 600, to 500, or to 450; from 450 to 2000, to 1750, to 1500, to 1250, to 1000, to 900, to 800, to 700, to 600, or to 500; from 500 to 2000, to 1750, to 1500, to 1250, to 1000, to 900, to 800, to 700, or to 600; from 600 to 2000, to 1750, to 1500, to 1250, to 1000, to 900, to 800, or to 700; from 700 to 2000, to 1750, to 1500, to 1250, to 1000, to 900, or to 800; from 800 to 2000, to 1750, to 1500, to 1250, to 1000, or to 900; from 900 to 2000, to 1750, to 1500, to 1250, or to 1000; from 1000 to 2000, to 1750, to 1500, or to 1250; from 1250 to 2000, to 1750, or to 1500; from 1500 to 2000, or to 1750; or from 1750 to 2000 amino acid residues.

In some embodiments, the silk or silk or silk-like proteins comprise greater than 2, greater than 4, greater than 6, greater than 8, greater than 10, greater than 12, greater than 14, greater than 16, greater than 18, greater than 20, greater than 22, greater than 24, greater than 26, greater than 28, or greater than 30; from 2 to 30, to 28, to 26, to 24, to 22, to 20, to 18, to 16, to 14, to 12, to 10, to 8, to 6, or to 4; from 4 to 30, to 28, to 26, to 24, to 22, to 20, to 18, to 16, to 14, to 12, to 10, to 8, or to 6; from 6 to 30, to 28, to 26, to 24, to 22, to 20, to 18, to 16, to 14, to 12, to 10, or to 8; from 8 to 30, to 28, to 26, to 24, to 22, to 20, to 18, to 16, to 14, to 12, or to 10; from 10 to 30, to 28, to 26, to 24, to 22, to 20, to 18, to 16, to 14, or to 12; from 12 to 30, to 28, to 26, to 24, to 22, to 20, to 18, to 16, or to 14; from 14 to 30, to 28, to 26, to 24, to 22, to 20, to 18, or to 16; from 16 to 30, to 28, to 26, to 24, to 22, to 20, or to 18; from 18 to 30, to 28, to 26, to 24, to 22, or to 20; from 20 to 30, to 28, to 26, to 24, or to 22; from 22 to 30, to 28, to 26, or to 24; from 24 to 30, to 28, or to 26; from 26 to 30, or to 28; from 28 to 30 exact-repeat and/or quasi-repeat units that each have molecular weights of greater than 5 kDa, greater than 10 kDa, greater than 20 kDa, greater than 30 kDa, greater than 40 kDa, greater than 50 kDa, greater than 60 kDa, greater than 70 kDa, greater than 80 kDa, or greater than 90 kDa; from 5 kDa to 100 kDa, to 90 kDa, to 80 kDa, to 70 kDa, to 60 kDa, to 50 kDa, to 40 kDa, to 30 kDa, to 20 kDa, or to 10 kDa; from 10 kDa to 100 kDa, to 90 kDa, to 80 kDa, to 70 kDa, to 60 kDa, to 50 kDa, to 40 kDa, to 30 kDa, or to 20 kDa; from 20 kDa to 100 kDa, to 90 kDa, to 80 kDa, to 70 kDa, to 60 kDa, to 50 kDa, to 40 kDa, or to 30 kDa; from 30 kDa to 100 kDa, to 90 kDa, to 80 kDa, to 70 kDa, to 60 kDa, to 50 kDa, or to 40 kDa; from 40 kDa to 100 kDa, to 90 kDa, to 80 kDa, to 70 kDa, to 60 kDa, or to 50 kDa; from 50 kDa to 100 kDa, to 90 kDa, to 80 kDa, to 70 kDa, or to 60 kDa; from 60 kDa to 100 kDa, to 90 kDa, to 80 kDa, or to 70 kDa; from 70 kDa to 100 kDa, to 90 kDa, or to 80 kDa; from 80 kDa to 100 kDa, or to 90 kDa; or from 90 kDa to 100 kDa. In some such embodiments, the order of the 2 or more exact-repeat or quasi-repeat units within the silk or silk or silk-like proteins is not native.

In some embodiments, the silk or silk or silk-like proteins comprise more than 1, more than 2, more than 4, more than 6, more than 8, more than 10, more than 15, more than 20, or more than 25; from 1 to 30, to 25, to 20, to 15, to 10, to 8, to 6, to 4, or to 2; from 2 to 30, to 25, to 20, to 15, to 10, to 8, to 6, or to 4; from 4 to 30, to 25, to 20, to 15, to 10, to 8, or to 6; from 6 to 30, to 25, to 20, to 15, to 10, or to 8; from 8 to 30, to 25, to 20, to 15, or to 10; from 10 to 30, to 25, to 20, or to 15; from 15 to 30, to 25, or to 20; from 20 to 30, or to 25; or from 25 to 30 exact-repeat and/or quasi-repeat units that are glycine-rich. In some such embodiments, one or more of the glycine-rich exact-repeat and/or quasi-repeat units comprise more than 4, more than 6, more than 8, more than 10, more than 12, more than 15, more than 18, more than 20, more than 25, more than 30, more than 40, more than 50, more than 60, more than 70, more than 80, more than 90, more than 100, or more than 150; from 4 to 200, to 150, to 100, to 90, to 80, to 70, to 60, to 50, to 40, to 30, to 25, to 20, to 18, to 15, to 12, to 10, to 8, or to 6; from 6 to 200, to 150, to 100, to 90, to 80, to 70, to 60, to 50, to 40, to 30, to 25, to 20, to 18, to 15, to 12, to 10, or to 8; from 8 to 200, to 150, to 100, to 90, to 80, to 70, to 60, to 50, to 40, to 30, to 25, to 20, to 18, to 15, to 12, or to 10; from 10 to 200, to 150, to 100, to 90, to 80, to 70, to 60, to 50, to 40, to 30, to 25, to 20, to 18, to 15, or to 12; from 12 to 200, to 150, to 100, to 90, to 80, to 70, to 60, to 50, to 40, to 30, to 25, to 20, to 18, or to 15; from 15 to 200, to 150, to 100, to 90, to 80, to 70, to 60, to 50, to 40, to 30, to 25, to 20, or to 18; from 18 to 200, to 150, to 100, to 90, to 80, to 70, to 60, to 50, to 40, to 30, to 25, or to 20; from 20 to 200, to 150, to 100, to 90, to 80, to 70, to 60, to 50, to 40, to 30, or to 25; from 25 to 200, to 150, to 100, to 90, to 80, to 70, to 60, to 50, to 40, or to 30; from 30 to 200, to 150, to 100, to 90, to 80, to 70, to 60, to 50, or to 40; from 40 to 200, to 150, to 100, to 90, to 80, to 70, to 60, or to 50; from 50 to 200, to 150, to 100, to 90, to 80, to 70, or to 60; from 60 to 200, to 150, to 100, to 90, to 80, or to 70; from 70 to 200, to 150, to 100, to 90, or to 80; from 80 to 200, to 150, to 100, or to 90; from 90 to 200, to 150, or to 100; from 100 to 200, or to 150; or from 150 to 200 consecutive amino acids that are more than 30%, more than 40%, more than 45%, more than 50%, more than 55%, more than 60%, more than 70%, or more than 80%; from 30% to 100%, to 90%, to 80%, to 70%, to 60%, to 55%, to 50%, to 45%, or to 40%; from 40% to 100%, to 90%, to 80%, to 70%, to 60%, to 55%, to 50%, or to 45%; from 45% to 100%, to 90%, to 80%, to 70%, to 60%, to 55%, or to 50%; from 50% to 100%, to 90%, to 80%, to 70%, to 60%, or to 55%; from 55% to 100%, to 90%, to 80%, to 70%, or to 60%; from 60% to 100%, to 90%, to 80%, or to 70%; from 70% to 100%, to 90%, or to 80%; from 80% to 100%, or to 90%; or from 90% to 100% glycine.

In some embodiments, the silk or silk or silk-like proteins comprise more than 1, more than 2, more than 4, more than 6, more than 8, more than 10, more than 15, more than 20, or more than 25; from 1 to 30, to 25, to 20, to 15, to 10, to 8, to 6, to 4, or to 2; from 2 to 30, to 25, to 20, to 15, to 10, to 8, to 6, or to 4; from 4 to 30, to 25, to 20, to 15, to 10, to 8, or to 6; from 6 to 30, to 25, to 20, to 15, to 10, or to 8; from 8 to 30, to 25, to 20, to 15, or to 10; from 10 to 30, to 25, to 20, or to 15; from 15 to 30, to 25, or to 20; from 20 to 30, or to 25; or from 25 to 30 exact-repeat and/or quasi-repeat units that are alanine-rich. In some such embodiments, one or more of the alanine-rich exact-repeat and/or quasi-repeat units comprise more than 4, more than 6, more than 8, more than 10, more than 12, more than 15, or more than 18; from 4 to 20, to 18, to 15, to 12, to 10, to 8, or to 6; from 6 to 20, to 18, to 15, to 12, to 10, or to 8; from 8 to 20, to 18, to 15, to 12, or to 10; from 10 to 18, to 15, or to 12; from 12 to 20, to 18, or to 15; from 15 to 20, or to 18; or from 18 to 20; consecutive amino acids that are more than 70%, more than 75%, more than 80%, more than 85%, or more than 90%; from 70% to 100%, to 90%, to 85%, to 80%, or to 75%; from 75% to 100%, to 90%, to 85%, or to 80%; from 80% to 100%, to 90%, or to 85%; from 85% to 100%, or to 90%; or from 90% to 100% alanine.

In some embodiments, the silk or silk or silk-like proteins comprise one or more glycine-rich exact-repeat and/or quasi-repeat units that are from 20 to 100 amino acids long and that are concatenated with poly-alanine-rich regions that are from 4 to 20 amino acids long. In some embodiments, the silk or silk or silk-like proteins comprise 5-25% poly-alanine regions (from 4 to 20 poly-alanine residues). In some embodiments, the silk or silk or silk-like proteins comprise 25-50% glycine. In some embodiments, the silk or silk or silk-like proteins comprise 15-35% GGX, where X is any amino acid. In some embodiments, the silk or silk or silk-like proteins comprise 15-60% GPG. In some embodiments, the silk or silk or silk-like proteins comprise 10-40% alanine. In some embodiments, the silk or silk or silk-like proteins comprise 0-20% proline. In some embodiments, the silk or silk or silk-like proteins comprise 10-50% beta-turns. In some embodiments, the silk or silk or silk-like proteins comprise 10-50% alpha-helix composition. In some embodiments, all of these compositional ranges apply to the same silk or silk or silk-like protein. In some embodiments, 2 or more of these compositional ranges apply to the same silk or silk or silk-like protein.

In some embodiments, the structure of the silk or silk or silk-like proteins form beta-sheet structures, beta-turn structures, or alpha-helix structures. In some embodiments, the secondary, tertiary, and quaternary structures of the silk or silk or silk-like proteins have nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. In some embodiments, the silk or silk or silk-like proteins are highly crystalline. In other embodiments, the silk or silk or silk-like proteins are highly amorphous. In some embodiments, the silk or silk or silk-like proteins comprise both crystalline and amorphous regions. In some embodiments, the silk or silk or silk-like proteins comprise from 10% to 40% crystalline material by volume.

In some embodiments, the silk or silk or silk-like proteins comprise one or more exact-repeat or quasi-repeat units that have at least 80%, at least 85%, at least 90%, at least 95%, or at least 99% amino acid sequence identity to a repeat unit of a native spider silk protein. In some embodiments, the silk or silk or silk-like proteins comprise one or more exact-repeat or quasi-repeat units that have an at least 80%, at least 85%, at least 90%, at least 95%, or at least 99% amino acid sequence identity to a repeat unit of a native spider dragline silk protein. In some embodiments, the silk or silk or silk-like proteins comprise one or more exact-repeat or quasi-repeat units that have at least 80%, at least 85%, at least 90%, at least 95%, or at least 99% amino acid sequence identity to a repeat unit of a native MA dragline silk protein. In some embodiments, the silk or silk or silk-like proteins comprise one or more exact-repeat or quasi-repeat units that have at least 80%, at least 85%, at least 90%, at least 95%, or at least 99% amino acid sequence identity to a repeat unit of a native MaSp2 dragline silk protein.

In some embodiments, the silk or silk or silk-like proteins comprise one or more quasi-repeat units, wherein the amino acid sequence of each quasi-repeat unit is described by Equation 1, wherein the amino acid sequence of X1 (termed a “motif”) is described by Equation 2 and can vary randomly within each quasi-repeat unit. The sequence [GPG-X1]ₙ₁ (SEQ ID NO: 147) is referred to as “first region”, and is glycine-rich. The sequence (A)ₙ₂ (SEQ ID NO: 148) is referred to as “second region”, and is alanine-rich. In some embodiments, the value of n1 is any one of 4, 5, 6, 7, or 8. In some embodiments, the value of n2 is any one of 6, 7, 8, 9, 10, 11, 12, 13, 14, 15, 16, 17, 18, 19, or 20. In some embodiments, the value of n3 is any one from 2 to 20. In some embodiments, the silk or silk or silk-like proteins comprise one or more of quasi-repeat units that have at least 80%, 90%, 95%, or 99% sequence identity to a quasi-repeat unit described by Equations 1 and 2.

(Equation 1)
(SEQ ID NO: 149)
{GGY - [GPG-X1]ₙ₁ - GPS-(A)ₙ₂}ₙ₃
(Equation 2)
X1 =
(SEQ ID NO: 150)
SGGQQ
or
(SEQ ID NO: 151)
GAGQQ
or
(SEQ ID NO: 152)
GQGPY
or
(SEQ ID NO: 153)
AGQQ
or
SQ

In some embodiments, the silk or silk or silk-like proteins comprise quasi-repeat units as described by Equation 1 and Equation 2, wherein n1 is 4 or 5 for at least half of the quasi-repeat units. In some embodiments, the silk or silk or silk-like proteins comprise quasi-repeat units as described by Equation 1 and Equation 2, wherein n2 is from 5 to 8 for at least half of the quasi-repeat units.

The term “short quasi-repeat unit” as used herein refers to a repeat unit in which n1 is 4 or 5 (as shown in Equation 1). The term “long quasi-repeat unit” as used herein refers to a repeat in which n1 is 6, 7, or 8 (as shown in Equation 1). In some embodiments, n1 is from 4 to 5 for at least half of the quasi-repeat units. In some embodiments, n2 is from 5 to 8 for at least half of the quasi-repeat units. In some embodiments, the silk or silk or silk-like proteins comprise 3 “long quasi-repeat units” followed by 3 “short quasi-repeat units”. In some embodiments, the silk or silk or silk-like proteins comprise quasi-repeat units that do not have the same X1 motifs more than twice in a row, or more than 2 times, in a single quasi-repeat. In some embodiments, the silk or silk or silk-like proteins comprise quasi-repeat units that comprise the same X1 motifs in the same location. In some embodiments, the silk or silk or silk-like proteins comprise quasi-repeat units that comprise the same Equation 2 sequence in the same location. In some embodiments, the silk or silk or silk-like proteins comprise quasi-repeat units wherein no more than 3 quasi-repeat units out of 6 share the same X1.

In some embodiments, the silk or silk or silk-like proteins comprise Xqr quasi-repeat units, wherein

$$Xqr = Xsqr + Xlqr , \qquad ( Equation 3 )$$

wherein Xqr is a number from 2 to 20; Xsqr is the number of short quasi-repeats, and a number from 1 to (Xqr-1); and Xlqr is the number of long quasi-repeats, and a number from 1 to (Xqr-1). In some embodiments, X_{qr} is a number from 2 to 20. Non-limiting examples of amino acid sequences of repeat units are given in Table 3.

TABLE 3
Exemplary Repeat Units of Silk or Silk-Like Proteins

| SEQ | |
|---|---|
| ID | |
| NO | Amino Acid Sequence |
| 17 | GGYGPGAGQQGPGSGGQQGPGGQGPYGSGQQGPGGAGQQGPGGQGPYGPGAAAAAAAAAGGYGPGAGQQGPGGAGQQGP |
| | GSQGPGGQGPYGPGAGQQGPGSQGPGSGGQQGPGGQGPYGPSAAAAAAAAAGGYGPGAGQRSQGPGGQGPYGPGAGQQGPG |
| | SQGPGSGGQQGPGGQGPYGPSAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGGQGPYGPSAAAAAAAA |
| 18 | GGQGGRGGFGGLGSQGAGGAGQGGAGAAAAAAAAGGDGGSGLGGYGAGRGHGVGLGGAGGAGAASAAAAAGGQGGRGG |
| | FGGLGSQGAGGAGQGGAGAAAAAAAAGGDGGSGLGGYGAGRGHGAGLGGAGGAGAASAAAAAGGQGGRGGFGGLGSQGS |
| | GGAGQGGSGAAAAAAAAGGDGGSGLGGYGAGRGYGAGLGGAGGAGAASAAAAAGGQGGRGGFGGLGSQGAGGAGQGGS |
| | GAAAAAAAAVADGGSGLGGYGAGRGYGAGLGGAGGAGAASAAAAT |
| 19 | GSAPQGAGGPAPQGPSQQGPVSQGPYGPGAAAAAAAAGGYGPGAGQQGPGSQGPGSGGQQGPGSQGPGSGGQQGPGGQGPY |
| | GPSAAAAAAAAAGGYGPGAGQQGPGSQGPGSGGQQGPGGQGPYGPGAAAAAAAVGGYGPGAGQQGPGSQGPGSGGQQGPG |
| | GQGPYGPSAAAAAAAAGGYGPGAGQQGPGSQGPGSGGQQGPGGQGPYGPSAAAAAAAAGGYGPGAGQQGPGSGGQQGPGG |
| | QGPYGSGQQGPGGAGQQGPGGQGPYGPGAAAAAAAAA |
| 20 | GGYGPGAGQQGPGSGGQQGPGGQGPYGSGQQGPGGAGQQGPGGQGPYGPGAAAAAAAAAGGYGPGAGQQGPGGAGQQGP |
| | GSQGPGGQGPYGPGAGQQGPGSQGPGSGGQQGPGGQGPYGPSAAAAAAAAGGYGPGAGQQGPGSQGPGSGGQQGPGGQGPY |
| | GPSAAAAAAAAGGYGPGAGQQGPGSGGQQGPGGQGPYGSGQQGPGGAGQQGPGGQGPYGGGYGPGAGQQGPGSQGPGSGG |
| | QQGPGGQGPYGPSAAAAAAAA |
| 21 | GPGARRQGPGSQGPGSGGQQGPGGQGPYGSGQQGPGGAGQQGPGGQGPYGPGAAAAAAAAAGGYGPGAGQQGPGGAGQQG |
| | PGSQGPGGQGPYGPGAGQQGPGSQGPGSGGQQGPGGQGPYGPSAAAAAAAAAGGYGPGAGQQGPGSQGPGSGGQQGPGGQG |
| | PYGPGAAAAAAAVGGYGPGAGQQGPGSQGPGSGGQQGPGGQGPYGPSAAAAAAAAGGYGPGAGQQGPGSQGPGSGGQQGP |
| | GGQGPYGPSAAAAAAAA |
| 22 | GPGARRQGPGSQGPGSGGQQGPGGQGPYGSGQQGPGGAGQQGPGGQGPYGPGAAAAAAAAAGGYGPGAGQQGPGGAGQQG |
| | PGSQGPGGQGPYGPGAGQQGPGSQGPGSGGQQGPGGQGPYGPSAAAAAAAAGGYGPGAGQQGPGSQGPGSGGQQGPGGQGP |
| | YGPGAAAAAAAVGGYGPGAGQQGPGSQGPGSGGQQGPGGQGPYGPSAAAAAAAAGGYGPGAGQQGPGSQGPGSGGQQGPG |
| | GQGPYGPSAAAAAAAA |
| 23 | GGYGPGAGQQGPGSGGQQGPGGQGPYGSGQQGPGGAGQQGPGGQGPYGPGAAAAAAAAAGGYGPGAGQQGPGGAGQQGPE |
| | GPGSQGPGSGGQQGPGGQGPYGPGAAAAAAAVGGYGPGAGQQGPGSQGPGSGGQQGPGGQGPYGPSAAAAAAAAGGYGPG |
| | AGQQGPGSQGPGSGGQQGPGGQGPYGPSAAAAAAAAGGYGPGAGQQGPGSGGQQGPGGQGPYGSGQQGPGGAGQQGPGGQ |
| | GPYGPGAAAAAAAAA |
| 24 | GVFSAGQGATPWENSQLAESFISRFLRFIGQSGAFSPNQLDDMSSIGDTLKTAIEKMAQSRKSSKSKLQALNMAFASSMAEIAVA |
| | EQGGLSLEAKTNAIASALSAAFLETTGYVNQQFVNEIKTLIFMIAQASSNEISGSAAAAGGSSGGGGGSGQGGYGQGAYASASA |
| | AAAYGSAPQGTGGPASQGPSQQGPVSQPSYGPSATVAVTAVGGRPQGPSAPRQQGPSQQGPGQQGPGGRGPYGPSAAAAAAA |
| | A |
| 25 | GAGAGAGAGAGAGAGAGSGASTSVSTSSSSGSGAGAGAGSGAGSGAGAGSGAGAGAGAGGAGAGFGSGLGLGYGVGLSSAQ |
| | AQAQAQAAAQAQAQAQAQAYAAAQAQAQAQAQAQAAAAAAAAAAAGAGAGAGAGAGAGAGAGSGASTSVSTSSSSGSGA |
| | GAGAGSGAGSGAGAGSGAGAGAGAGGAGAGFGSGLGLGYGVGLSSAQAQAQAQAAAQAQAQAQAQAYAAAQAQAQAQA |
| | QAQAAAAAAAAAAA |
| 26 | GAGAGAGAGAGAGAGAGSGASTSVSTSSSSGSGAGAGAGSGAGSGAGAGSGAGAGAGAGGAGAAFGSGLGLGYGVGLSSAQ |
| | AQAQAQAAAQAQADAQAQAYAAAQAQAQAQAQAQAAAAAAAAAAAGAGAGAGAGSGAGAGAGSGASTSVSTSSSSGSGA |
| | GAGAGSGAGSGAGAGSGAGAGAGAGGAGAGFGSGLGLGYGVGLSSAQAQAQAQAAAQAQADAQAQAYAAAQAQAQAQA |
| | QAQAAAAAAAAAAA |
| 27 | GAGAGAGAGSGAGAGAGSGASTSVSTSSSSGSGAGAGAGSGAGSGAGAGSGAGAGAGAGGAGAGFGSGLGLGYGVGLSSAQ |
| | AQAQSAAAARAQADAQAQAYAAAQAQAQAQAQAQAAAAAAAAAAAGAGAGAGAGAGAGAGAGSGASTSVSTSSSSASGA |
| | GAGAGSGAGSGAGAGSGAGAGAGAGGAGAGFGSGLGLGYGVGLSSAQAQAQAQAAAQAQAQAQAQALAAAQAQAQAQAQ |
| | AQAAAATAAAAAA |
| 28 | GGYGPGAGQQGPGGAGQQGPGSQGPGGQGPYGPGAGQQGPGSQGPGSGGQQGPGGQGPYGPSAAAAAAAAGGYGPGAGQQ |
| | GPGSQGPGSGGQQGPGSQGPGSGGQQGPGGQGPYGPSAAAAAAAAAGGYGPGAGQQGPGSQGPGSGGQQGPGGQGPYGPGA |
| | AAAAAAVGGYGPGAGQQGPGSQGPGSGGQQGPGGQGPYGPSAAAAAAAAGGYGPGAGQQGPGSQGPGSGGQQGPGGQGPY |
| | GPSAAAAAAAA |
| 29 | GGYGPGAGQQGPGGAGQQGPGSQGPGGQGPYGPGAGQQGPGSQGPGSGGQQGPGGQGPYGPSAAAAAAAAGGYGPGAGQQ |
| | GPGSQGPGSGGQQGPGSQGPGSGGQQGPGGQGPYGPSAAAAAAAAGGYGPGAGQQGPGSQGPGSGGQQGPGGQGPYGPGAA |
| | AAAAAVGGYGPGAGQQGPGSQGPGSGGQQGPGGQGPYGPSAAAAAAAAGGYGPGAGQQGPGSQGPGSGGQQGPGGQGPYG |
| | PSAAAAAAAA |
| 30 | GHQGPHRKTPWETPEMAENFMNNVRENLEASRIFPDELMKDMEAITNTMIAAVDGLEAQHRSSYASLQAMNTAFASSMAQLF |
| | ATEQDYVDTEVIAGAIGKAYQQITGYENPHLASEVTRLIQLFREEDDLENEVEISFADTDNAIARAAAGAAAGSAAASSSADASA |
| | TAEGASGDSGFLFSTGTFGRGGAGAGAGAAAASAAAASAAAAGAEGDRGLFFSTGDFGRGGAGAGAGAAAASAAAASAAAA |
| 31 | GGAQKHPSGEYSVATASAAATSVTSGGAPVGKPGVPAPIFYPQGPLQQGPAPGPSNVQPGTSQQGPIGGVGESNTFSSSFASALG |
| | GNRGFSGVISSASATAVASAFQKGLAPYGTAFALSAASAAADAYNSIGSGASASAYAQAFARVLYPLLQQYGLSSSADASAFAS |
| | AIASSFSTGVAGQGPSVPYVGQQQPSIMVSAASASAAASAAAVGGGPVVQGPYDGGQPQQPNIAASAAAAATATSS |
| 32 | GGQGGRGGFGGLGSQGEGGAGQGGAGAAAAAAAAGADGGFGLGGYGAGRGYGAGLGGAGGAGAASAAAAAGGQGGRSGF |
| | GGLGSQGAGGAGQGGAGAAAAAAAAGADGGSGLGGYGAGRGYGASLGGADGAGAASAAAAAGGQGGRGGFGGLGSQGAG |
| | GAGQGGAGAAAAAAAASGDGGSGLGGYGAGRGYGAGLGGAGGAGAASAAAAAGGEGGRGGFGGLGSQGAGGAGQGGSLA |
| | AAAAAAA |
| 33 | GPGGYGGPGQPGPGQGQYGPGPGQQGPRQGGQQGPASAAAAAAAGPGGYGGPGQQGPRQGQQQGPASAAAAAAAAAAGPR |
| | GYGGPGQQGPVQGGQQGPASAAAAAAAAGVGGYGGPGQQGPGQGQYGPGTGQQGQGPSGQQGPAGAAAAAAGGAAGPGG |
| | YGGPGQQGPGQGQYGPGTGQQGQGPSGQQGPAGAAAAAAAAAGPGGYGGPGQQGPGQGQYGPGAGQQGQGPGSQQGPASA |
| | AAAAA |
| 34 | GSGAGQGTGAGAGAAAAAAGAAGSGAGQGAGSGAGAAAAAAAASAAGAGQGAGSGSGAGAAAAAAAAAGAGQGAGSGS |
| | GAGAAAAAAAAAAAAQQQQQQQAAAAAAAAAAAAAGSGQGASFGVTQQFGAPSGAASSAAAAAAAAAAAAAGSGAGQEA |
| | GTGAGAAAAAAAAGAAGSGAGQGAGSGAGAAAAAAAAASAAGAGQGAGSGSGAGAAAAAAAAAAAAQQQQQQQAAAAA |
| | AAAAAAAA |
| 35 | GGAQKQPSGESSVATASAAATSVTSAGAPVGKPGVPAPIFYPQGPLQQGPAPGPSYVQPATSQQGPIGGAGRSNAFSSSFASALS |
| | GNRGFSEVISSASATAVASAFQKGLAPYGTAFALSAASAAADAYNSIGSGANAFAYAQAFARVLYPLVQQYGLSSSAKASAFAS |
| | AIASSFSSGAAGQGQSIPYGGQQQPPMTISAASASAGASAAAVKGGQVGQGPYGGQQQSTAASASAAATTATA |
| 36 | GADGGSGLGGYGAGRGYGAGLGGADGAGAASAAAAAGGQGGRGGFGRLGSQGAGGAGQGGAGAAAAVAAAGGDGGSGL |
| | GGYGAGRGYGAGLGGAGGAGAASAAAAAGGQGGRGGFGGLGSQGAGGAGQGGAGAAASGDGGSGLGGYGAGRGYGAGL |
| | GGADGAGAASAASAAGGQGGRGGFGGLGSQGAGGAGQGGAGAAAAAATAGGDGGSGLGGYGAGRGYGAGLGGAGGAGA |
| | ASAAAAA |
| 37 | GAGAGQGGRGGYGQGGFGGQGSGAGAGASAAAGAGAGQGGRGGYGQGGFGGQGSGAGAGASAAAGAGAGQGGRGGYGQ |
| | GGFGGQGSGAGAGASAAAAAGAGQGGRGGYGQGGLGGSGSGAGAGAGAAAAAAAGAGGYGQGGLGGYGQGAGAGQGGL |
| | GGYGSGAGAGASAAAAAGAGGAGQGGLGGYGQGAGAGQGGLGGYGSGAGAGAAAAAAAGAGGSGQGGLGGYGSGGGAG |
| | GASAAAA |
| 38 | GAYAYAYAIANAFASILANTGLLSVSSAASVASSVASAIATSVSSSSAAAAASASAAAAASAGASAASSASASSSASAAAGAGAG |
| | AGAGASGASGAAGGSGGFGLSSGFGAGIGGLGGYPSGALGGLGIPSGLLSSGLLSPAANQRIASLIPLILSAISPNGVNFGVIGSNIA |
| | SLASQISQSGGGIAASQAFTQALLELVAAFIQVLSSAQIGAVSSSSASAGATANAFAQSLSSAFAG |
| 39 | GAAQKQPSGESSVATASAAATSVTSGGAPVGKPGVPAPIFYPQGPLQQGPAPGPSNVQPGTSQQGPIGGVGGSNAFSSSFASALS |
| | LNRGFTEVISSASATAVASAFQKGLAPYGTAFALSAASAAADAYNSIGSGANAFAYAQAFARVLYPLVRQYGLSSSGKASAFAS |
| | AIASSFSSGTSGQGPSIGQQQPPVTISAASASAGASAAAVGGGQVGQGPYGGQQQSTAASASAAAATATS |
| 40 | GAAQKQPSGESSVATASAAATSVTSGGAPVGKPGVPAPIFYPQGPLQQGPAPGPSNVQPGTSQQGPIGGVGGSNAFSSSFASALS |
| | LNRGFTEVISSASATAVASAFQKGLAPYGTAFALSAASAAADAYNSIGSGANAFAYAQAFARVLYPLVRQYGLSSSGKASAFAS |
| | AIASSFSSGTSGQGPSIGQQQPPVTISAASASAGASAAAVGGGQVGQGPYGGQQQSTAASASAAAATATS |
| 41 | GAAQKQPSGESSVATASAAATSVTSGGAPVGKPGVPAPIFYPQGPLQQGPAPGPSNVQPGTSQQGPIGGVGGSNAFSSSFASALS |
| | LNRGFTEVISSASATAVASAFQKGLAPYGTAFALSAASAAADAYNSIGSGANAFAYAQAFARVLYPLVQQYGLSSSAKASAFAS |
| | AIASSFSSGTSGQGPSIGQQQPPVTISAASASAGASAAAVGGGQVGQGPYGGQQQSTAASASAAAATATS |
| 42 | GGAQKQPSGESSVATASAAATSVTSAGAPVGKPGVPAPIFYPQGPLQQGPAPGPSNVQPGTSQQGPIGGVGGSNAFSSSFASALS |
| | LNRGFTEVISSASATAVASAFQKGLAPYGTAFALSAASAAADAYNSIGSGANAFAYAQAFARVLYPLVQQYGLSSSAKASAFAS |
| | AIASSFSSGTSGQGPSNGQQQPPVTISAASASAGASAAAVGGGQVSQGPYGGQQQSTAASASAAAATATS |
| 43 | GGAQKQPSGESSVATASAAATSVTSAGAPGGKPGVPAPIFYPQGPLQQGPAPGPSNVQPGTSQQGPIGGVGGSNAFSSSFASALS |
| | LNRGFTEVISSASATAVASAFQKGLAPYGTAFALSAASAAADAYNSIGSGANAFAYAQAFARVLYPLVQQYGLSSSAKASAFAS |
| | AIASSFSSGTSGQGPSIGQQQPPVTISAASASAGASAAAVGGGQVGQGPYGGQQQSTAASASAAAATATS |
| 44 | GPGGYGGPGQQGPGQGQQQGPASAAAAAAAAGPGGYGGPGQQGPGQGQQQGPASAAAAAAAAAGPGGYGGPGQQRPGQA |
| | QYGRGTGQQGQGPGAQQGPASAAAAAAAGAGLYGGPGQQGPGQGQQQGPASAAAAAAAAAAAGPGGYGGPGQQGPGQAQ |
| | QQGPASAAAAAAAGPGGYSGPGQQGPGQAQQQGPASAAAAAAAAAGPGGYGGPGQQGPGQGQQQGPASAAAAAAATAA |
| 45 | GAGGDGGLFLSSGDFGRGGAGAGAGAAAASAAAASSAAAGARGGSGFGVGTGGFGRGGAGDGASAAAASAAAASAAAAGA |
| | GGDSGLFLSSGDFGRGGAGAGAGAAAASAAAASAAAAGTGGVGGLFLSSGDFGRGGAGAGAGAAAASAAAASSAAAGARGG |
| | SGFGVGTGGFGRGGPGAGTGAAAASAAAASAAAAGAGGDSGLFLSSEDFGRGGAGAGTGAAAASAAAASAAAA |
| 46 | GAGRGYGGGYGGGAAAGAGAGAGAGRGYGGGYGGGAGSGAGSGAGAGGGSGYGRGAGAGAGAGAAAAAGAGAGGAGG |
| | YGGGAGAGAGASAAAGAGAGAGGAGGYGGGYGGGAGAGAGAGAAAAAGAGAGAGAGRGYGGGFGGGAGSGAGAGAGA |
| | GGGSGYGRGAGGYGGGYGGGAGTGAGAAAATGAGAGAGAGRGYGGGYGGGAGAGAGAGAGAGGGSGYGRGAGAGASVA |
| | A |
| 47 | GALGQGASVWSSPQMAENFMNGFSMALSQAGAFSGQEMKDFDDVRDIMNSAMDKMIRSGKSGRGAMRAMNAAFGSAIAEIV |
| | AANGGKEYQIGAVLDAVTNTLLQLTGNADNGFLNEISRLITLFSSVEANDVSASAGADASGSSGPVGGYSSGAGAAVGQGTAQ |
| | AVGYGGGAQGVASSAAAGATNYAQGVSTGSTQNVATSTVTTTTNVAGSTATGYNTGYGIGAAAGAAA |
| 48 | GGQGGQGGYDGLGSQGAGQGGYGQGGAAAAAAAASGAGSAQRGGLGAGGAGQGYGAGSGGQGGAGQGGAAAATAAAAG |
| | GQGGQGGYGGLGSQGSGQGGYGQGGAAAAAAAASGDGGAGQEGLGAGGAGQGYGAGLGGQGGAGQGGAAAAAAAAAGG |
| | QGGQGGYGGLGSQGAGQGGYGQGGAAAAAAAASGAGGAGQGGLGAAGAGQGYGAGSGGQGGAGQGGAAAAAAAAA |
| 49 | GGQGGQGGYGGLGSQGAGQGGYGQGGVAAAAAAASGAGGAGRGGLGAGGAGQEYGAVSGGQGGAGQGGEAAAAAAAAG |
| | GQGGQGGYGGLGSQGAGQGGYGQGGAAAAAAAASGAGGARRGGLGAGGAGQGYGAGLGGQGGAGQGSASAAAAAAAGG |
| | QGGQGGYGGLGSQGSGQGGYGQGGAAAAAAAASGAGGAGRGSLGAGGAGQGYGAGLGGQGGAGQGGAAAAASAAA |
| 50 | GPGGYGGPGQQGPGQGQYGPGTGQQGQGPGGQQGPVGAAAAAAAAVSSGGYGSQGAGQGGQQGSGQRGPAAAGPGGYSGP |
| | GQQGPGQGGQQGPASAAAAAAAAAGPGGYGGSGQQGPGQGRGTGQQGQGPGGQQGPASAAAAAAAGPGGYGGPGQQGPG |
| | QGQYGPGTGQQGQGPASAAAAAAAGPGGYGGPGQQGPGQGQYGPGTGQQGQGPGGQQGPGGASAAAAAAA |
| 51 | GGYGPGAGQQGPGSGGQQGPGGQGPYGSGQQGPGGAGQQGPGGQGPYGPGAAAAAAAAAGGYGPGAGQQGPGGAGQQGP |
| | GSQGPGGQGPYGPGAGQQGPGSQGPGSGGQQGPGGQGPYGPSAAAAAAAAAGGYGPGAGQRSQGPGGQGPYGPGAGQQGPG |
| | SQGPGSGGQQGPGGQGPYGPSAAAAAAAAGPGAGRQGPGSQGPGSGGQQGPGGQGPYGPSAAAAAAAA |
| 52 | GQGGQGGQGGLGQGGYGQGAGSSAAAAAAAAAAAAAAGRGQGGYGQGSGGNAAAAAAAAAAAASGQGSQGGQGGQGQG |
| | GYGQGAGSSAAAAAAAAAAAAASGRGQGGYGQGAGGNAAAAAAAAAAAAAAGQGGQGGYGGLGQGGYGQGAGSSAAAA |
| | AAAAAAAAGGQGGQGQGGYGQGSGGSAAAAAAAAAAAAAAAGRGQGGYGQGSGGNAAAAAAAAAAAAAA |
| 53 | GRGPGGYGPGQQGPGGPGAAAAAAGPGGYGPGGYGPGQQGPGGPGAAAAAAAGRGPGGYGPGQQGPGQQGPGGSGAAAAA |
| | AGRGPGGYGPGQQGPGGPGAAAAAAGPGGYGPGQQGPGAAAAAAAAGRGPGGYGPGQQGPGGPGAAAAAAAGRGPGGYGP |
| | GQQGPGQQGPGGSGAAAAAAGRGPGGYGPGQQGPGGPGAAAAAAGPGGYGPGQQGPGAAAAAAAA |
| 54 | GRGPGGYGPGQQGPGGSGAAAAAAGRGPGGYGPGQQGPGGPGAAAAAAGPGGYGPGQQGTGAAAAAAAGSGAGGYGPGQ |
| | QGPGGPGAAAAAAGPGGYGPGQQGPGAAAAAAAGSGPGGYGPGQQGPGGSSAAAAAAGPGRYGPGQQGPGAAAAASAGRG |
| | PGGYGPGQQGPGGPGAAAAAAGPGGYGPGQQGPGAAAAAAAGSGPGGYGPGQQGPGGPGAAAAAAA |
| 55 | GAAATAGAGASVAGGYGGGAGAAAGAGAGGYGGGYGAVAGSGAGAAAAASSGAGGAAGYGRGYGAGSGAGAGAGTVAA |
| | YGGAGGVATSSSSATASGSRIVTSGGYGYGTSAAAGAGVAAGSYAGAVNRLSSAEAASRVSSNIAAIASGGASALPSVISNIYSG |
| | VVASGVSSNEALIQALLELLSALVHVLSSASIGNVSSVGVDSTLNVVQDSVGQYVG |
| 56 | GGQGGFSGQGQGGFGPGAGSSAAAAAAAAAAARQGGQGQGGFGQGAGGNAAAAAAAAAAAAAAQQGGQGGFSGRGQGGF |
| | GPGAGSSAAAAAAGQGGQGQGGFGQGAGGNAAAAAAAAAAAAAAAGQGGQGRGGFGQGAGGNAAAAAAAAAAAAAAAQ |
| | QGGQGGFGGRGQGGFGPGAGSSAAAAAAGQGGQGRGGFGQGAGGNAAAASAAAAASAAAAGQ |
| 57 | GGYGPGAGQQGPGGAGQQGPGSQGPGGAGQQGPGGQGPYGPGAAAAAAAVGGYGPGAGQQGPGSQGPGSGGQQGPGGQGP |
| | YGPSAAAAAAAAGGYGPGAGQQGPGSQGPGSGGQQGPGGLGPYGPSAAAAAAAAGGYGPGAGQQGPGSQGPGSGGQQRPGG |
| | LGPYGPSAAAAAAAAGGYGPGAGQQGPGSQGPGSGGQQRPGGLGPYGPSAAAAAAAA |
| 58 | GAGAGGGYGGGYSAGGGAGAGSGAAAGAGAGRGGAGGYSAGAGTGAGAAAGAGTAGGYSGGYGAGASSSAGSSFISSSSMS |
| | SSQATGYSSSSGYGGGAASAAAGAGAAAGGYGGGYGAGAGAGAAAASGATGRVANSLGAMASGGINALPGVFSNIFSQVSAA |
| | SGGASGGAVLVQALTEVIALLLHILSSASIGNVSSQGLEGSMAIAQQAIGAYAG |
| 59 | GAGAGGAGGYAQGYGAGAGAGAGAGTGAGGAGGYGQGYGAGSGAGAGGAGGYGAGAGAGAGAGDASGYGQGYGDGAG |
| | AGAGAAAAAGAAAGARGAGGYGGGAGAGAGAGAGAAGGYGQGYGAGAGEGAGAGAGAGAVAGAGAAAAAGAGAGAGG |
| | AEGYGAGAGAGGAGGYGQSYGDGAAAAAGSGAGAGGSGGYGAGAGAGSGAGAAGGYGGGAGA |
| 60 | GPGGYGPGQQGPGGYGPGQQGPGRYGPGQQGPSGPGSAAAAAAGSGQQGPGGYGPRQQGPGGYGQGQQGPSGPGSAAAASA |
| | AASAESGQQGPGGYGPGQQGPGGYGPGQQGPGGYGPGQQGPSGPGSAAAAAAAASGPGQQGPGGYGPGQQGPGGYGPGQQG |
| | PSGPGSAAAAAAAASGPGQQGPGGYGPGQQGPGGYGPGQQGLSGPGSAAAAAAA |
| 61 | GRGPGGYGQGQQGPGGPGAAAAAAGPGGYGPGQQGPGAAAAAAAGSGPGGYGPGQQGPGRSGAAAAAAAAGRGPGGYGPG |
| | QQGPGGPGAAAAAAGPGGYGPGQQGPGAAAAASAGRGPGGYGPGQQGPGGSGAAAAAAGRGPGGYGPGQQGPGGPGAAAA |
| | AAAGRGPGGYGPGQQGPGQQGPGGSGAAAAAAGRGPGGYGPGQQGPGGPGAAAAAA |
| 62 | GVGAGGEGGYDQGYGAGAGAGSGGGAGGAGGYGGGAGAGSGGGAGGAGGYGGGAGAGAGAGAGGAGGYGGGAGAGTG |
| | ARAGAGGVGGYGQSYGAGASAAAGAGVGAGGAGAGGAGGYGQGYGAGAGIGAGDAGGYGGGAGAGASAGAGGYGGGAG |
| | AGAGGVGGYGKGYGAGSGAGAAAAAGAGAGSAGGYGRGDGAGAGGASGYGQGYGAGAAA |
| 63 | GYGAGAGRGYGAGAGAGAGAVAASGAGAGAGYGAGAGAGAGAGYGAGAGRGYGAGAGAGAGSGAASGAGAGAGYGAG |
| | AGAGAGYGAGAGSGYGTGAGAGAGAAAAGGAGAGAGYGAGAGRGYGAGAGAGAASGAGAGAGAGAASGAGAGSGYGAG |
| | AAAAGGAGAGAGGGYGAGAGRGYGAGAGAGAGAGSGSGSAAGYGQGYGSGSGAGAAA |
| 64 | GQGTDSSASSVSTSTSVSSSATGPDTGYPVGYYGAGQAEAAASAAAAAAASAAEAATIAGLGYGRQGQGTDSSASSVSTSTSVS |
| | SSATGPDMGYPVGNYGAGQAEAAASAAAAAAASAAEAATIASLGYGRQGQGTDSSASSVSTSTSVSSSATGPGSRYPVRDYGA |
| | DQAEAAASAAAAAAAAASAAEEIASLGYGRQ |
| 65 | GQGTDSVASSASSSASASSSATGPDTGYPVGYYGAGQAEAAASAAAAAAASAAEAATIAGLGYGRQGQGTDSSASSVSTSTSVS |
| | SSATGPGSRYPVRDYGADQAEAAASATAAAAAAASAAEEIASLGYGRQGQGTDSVASSASSSASASSSATGPDTGYPVGYYGA |
| | GQAEAAASAAAAAAASAAEAATIAGLGYGRQ |
| 66 | GQGGQGGYGGLGQGGYGQGAGSSAAAAAAAAAAAAAGGQGGQGQGRYGQGAGSSAAAAAAAAAAAAAAGRGQGGYGQG |
| | SGGNAAAAAAAAAAAASGQGSQGGQGGQGQGGYGQGAGSSAAAAAAAAAAAAASGRGQGGYGQGAGGNAAAAAAAAAA |
| | AAAAGQGGQGGYGGLGQGGYGQGAGSSAAAAAAAAAAAA |
| 67 | GGLGGQGGLGGLGSQGAGLGGYGQGGAGQGGAAAAAAAAGGLGGQGGRGGLGSQGAGQGGYGQGGAGQGGAAAAAAAA |
| | GGLGGQGGLGALGSQGAGQGGAGQGGYGQGGAAAAAAGGLGGQGGLGGLGSQGAGQGGYGQGGAGQGGAAAAAAAAGG |
| | LGGQGGLGGLGSQGAGPGGYGQGGAGQGGAAAAAAAA |
| 68 | GGQGRGGFGQGAGGNAAAAAAAAAAAAAAQQVGQFGFGGRGQGGFGPFAGSSAAAAAAASAAAGQGGQGQGGFGQGAGG |
| | NAAAAAAAAAAAARQGGQGQGGFSQGAGGNAAAAAAAAAAAAAAAQQGGQGGFGGRGQGGFGPGAGSSAAAAAAATAA |
| | AGQGGQGRGGFGQGAGSNAAAAAAAAAAAAAAAGQ |
| 69 | GGQGGQGGYGGLGSQGAGQGGYGAGQGAAAAAAAAGGAGGAGRGGLGAGGAGQGYGAGLGGQGGAGQAAAAAAAGGA |
| | GGARQGGLGAGGAGQGYGAGLGGQGGAGQGGAAAAAAAAGGQGGQGGYGGLGSQGAGQGGYGAGQGGAAAAAAAAGG |
| | QGGQGGYGGLGSQGAGQGGYGGRQGGAGAAAAAAAA |
| 70 | GGAGQRGYGGLGNQGAGRGGLGGQGAGAAAAAAAGGAGQGGYGGLGNQGAGRGGQGAAAAAGGAGQGGYGGLGSQGAG |
| | RGGQGAGAAAAAAVGAGQEGIRGQGAGQGGYGGLGSQGSGRGGLGGQGAGAAAAAAGGAGQGGLGGQGAGQGAGAAAA |
| | AAGGVRQGGYGGLGSQGAGRGGQGAGAAAAAA |
| 71 | GGAGQGGLGGQGAGQGAGASAAAAGGAGQGGYGGLGSQGAGRGGEGAGAAAAAAGGAGQGGYGGLGGQGAGQGGYGGL |
| | GSQGAGRGGLGGQGAGAAAAGGAGQGGLGGQGAGQGAGAAAAAAGGAGQGGYGGLGSQGAGRGGLGGQGAGAVAAAAA |
| | GGAGQGGYGGLGSQGAGRGGQGAGAAAAAA |
| 72 | GAGAGAGAGSGAGAAGGYGGGAGAGVGAGGAGGYDQGYGAGAGAGSGAGAGGAGGYGGGAGAGADAGAGGAGGYGGG |
| | AGAGAGARAGAGGVGGYGQSYGAGAGAGAGVGAGGAGAGGADGYGQGYGAGAGTGAGDAGGYGGGAGAGASAGAGGY |
| | GGGAGAGGVGVYGKGYGSGSGAGAAAAA |
| 73 | GGAGGYGVGQGYGAGAGAGAAAGAGAGGAGGYGAGQGYGAGAGVGAAAAAGAGAGVGGAGGYGRGAGAGAGAGAGAA |
| | AGAGAGAAAGAGAGGAGGYGAGQGYGAGAGVGAAAAAGAGAGVGGAGGYGRGAGAGAGAGAGGAGGYGRGAGAGAGA |
| | GAGAGGAGGYGAGQGYGAGAGAGAAAAA |
| 74 | GEAFSASSASSAVVFESAGPGEEAGSSGDGASAAASAAAAAGAGSGRRGPGGARSRGGAGAGAGAGSGVGGYGSGSGAGAGA |
| | GAGAGAGGEGGFGEGQGYGAGAGAGFGSGAGAGAGAGSGAGAGEGVGSGAGAGAGAGFGVGAGAGAGAGAGFGSGAGAG |
| | SGAGAGYGAGRAGGRGRGGRG |
| 75 | GEAFSASSASSAVVFESAGPGEEAGSSGGGASAAASAAAAAGAGSGRRGPGGARSRGGAGAGAGAGSGVGGYGSGSGAGAGA |
| | GAGAGAGGEGGFGEGQGYGAGAGAGFGSGAGAGAGAGSGAGAGEGVGSGAGAGAGAGFGVGAGAGAGAGAGFGSGAGAG |
| | SGAGAGYGAGRAGGRGRGGRG |
| 76 | GNGLGQALLANGVLNSGNYLQLANSLAYSFGSSLSQYSSSAAGASAAGAASGAAGAGAGAASSGGSSGSASSSTTTTTTTSTSA |
| | AAAAAAAAAAASAAASTSASASASASASASAFSQTFVQTVLQSAAFGSYFGGNLSLQSAQAAASAAAQAAAQQIGLGSYGYAL |
| | ANAVASAFASAGANA |
| 77 | GNGLGQALLANGVLNSGNYLQLANSLAYSFGSSLSQYSSSAAGASAAGAASGAAGAGAGAASSGGSSGSASSSTTTTTTTSTSA |
| | AAAAAAAAAAASAAASTSASASASASASASAFSQTFVQTVLQSAAFGSYFGGNLSLQSAQAAASAAAQAAAQQIGLGSYGYAL |
| | ANAVASAFASAGANA |
| 78 | GNGLGQALLANGVLNSGNYLQLANSLAYSFGSSLSQYSSSAAGASAAGAASGAAGAGAGAASSGGSSGSASSSTTTTTTTSTSA |
| | AAAAAAAAAAASAAASTSASASASASASASAFSQTFVQTVLQSAAFGSYFGGNLSLQSAQAAASAAAQAAAQQIGLGSYGYAL |
| | ANAVASAFASAGANA |
| 79 | GASGAGQGQGYGQQGQGGSSAAAAAAAAAAAAAAAQGQGQGYGQQGQGSAAAAAAAAAAGASGAGQGQGYGQQGQGSA |
| | AAAAAAAAAGASGAGQGQGYGQQGQGGSSAAAAAAAAAAAAAAAAQGQGYGQQGQGSAAAAAAAAAGASGAGQGQGYG |
| | QQGQGGSSAAAAAAAAAAAAAAAA |
| 80 | GRGQGGYGQGSGGNAAAAAAAGQGGFGGQEGNGQGAGSAAAAAAAAAAAAGGSGQGRYGGRGQGGYGQGAGAAASAAA |
| | AAAAAAAGQGGFGGQEGNGQGAGSAAAAAAAAAAAAGGSGQGGYGGRGQGGYGQGAGAAAAAAAAAAAAAAGQGGQG |
| | GFGSQGGNGQGAGSAAAAAAAAAA |
| 81 | GQNTPWSSTELADAFINAFMNEAGRTGAFTADQLDDMSTIGDTIKTAMDKMARSNKSSKGKLQALNMAFASSMAEIAAVEQG |
| | GLSVDAKTNAIADSLNSAFYQTTGAANPQFVNEIRSLINMFAQSSANEVSYGGGYGGQSAGAAASAAAAGGGGQGGYGNLGG |
| | QGAGAAAAAAASAA |
| 82 | GQNTPWSSTELADAFINAFLNEAGRTGAFTADQLDDMSTIGDTLKTAMDKMARSNKSSQSKLQALNMAFASSMAEIAAVEQGG |
| | LSVAEKTNAIADSLNSAFYQTTGAVNVQFVNEIRSLISMFAQASANEVSYGGGYGGGQGGQSAGAAAAAASAGAGQGGYGGL |
| | GGQGAGSAAAAAA |
| 83 | GGQGGQGGYGGLGSQGAGQGGYGQGGAAAAAASAGGQGGQGGYGGLGSQGAGQGGYGGGAFSGQQGGAASVATASAAAS |
| | RLSSPGAASRVSSAVTSLVSSGGPTNSAALSNTISNVVSQISSSNPGLSGCDVLVQALLEIVSALVHILGSANIGQVNSSGVGRSASI |
| | VGQSINQAFS |
| 84 | GGAGQGGYGGLGGQGAGAAAAAAGGAGQGGYGGQGAGQGAAAAAASGAGQGGYEGPGAGQGAGAAAAAAGGAGQGGY |
| | GGLGGQGAGQGAGAAAAAAGGAGQGGYGGLGGQGAGQGAGAAAAAAGGAGQGGYGGQGAGQGAAAAAAGGAGQGGYG |
| | GLGSGQGGYGRQGAGAAAAAAAA |
| 85 | GASSAAAAAAATATSGGAPGGYGGYGPGIGGAFVPASTTGTGSGSGSGAGAAGSGGLGGLGSSGGSGGLGGGNGGSGASAAA |
| | SAAAASSSPGSGGYGPGQGVGSGSGSGAAGGSGTGSGAGGPGSGGYGGPQFFASAYGGQGLLGTSGYGNGQGGASGTGSGGV |
| | GGSGSGAGSNS |
| 86 | GQPIWTNPNAAMTMTNNLVQCASRSGVLTADQMDDMGMMADSVNSQMQKMGPNPPQHRLRAMNTAMAAEVAEVVATSPP |
| | QSYSAVLNTIGACLRESMMQATGSVDNAFTNEVMQLVKMLSADSANEVSTASASGASYATSTSSAVSSSQATGYSTAAGYGNA |
| | AGAGAGAAAAVS |
| 87 | GQKIWTNPDAAMAMTNNLVQCAGRSGALTADQMDDLGMVSDSVNSQVRKMGANAPPHKIKAMSTAVAAGVAEVVASSPPQ |
| | SYSAVLNTIGGCLRESMMQVTGSVDNTFTTEMMQMVNMFAADNANEVSASASGSGASYATGTSSAVSTSQATGYSTAGGYGT |
| | AAGAGAGAAAAA |
| 88 | GSGYGAGAGAGAGSGYGAGAGAGSGYGAGAGAGAGSGYVAGAGAGAGAGSGYGAGAGAGAGSSYSAGAGAGAGSGYGA |
| | GSSASAGSAVSTQTVSSSATTSSQSAAAATGAAYGTRASTGSGASAGAAASGAGAGYGGQAGYGQGGGAAAYRAGAGSQAA |
| | YGQGASGSSGAAAAA |
| 89 | GGQGGRGGFGGLSSQGAGGAGQGGSGAAAAAAAAGGDGGSGLGDYGAGRGYGAGLGGAGGAGVASAAASAAASRLSSPSA |
| | ASRVSSAVTSLISGGGPTNPAALSNTFSNVVYQISVSSPGLSGCDVLIQALLELVSALVHILGSAIIGQVNSSAAGESASLVGQSVY |
| | QAFS |
| 90 | GVGQAATPWENSQLAEDFINSFLRFIAQSGAFSPNQLDDMSSIGDTLKTAIEKMAQSRKSSKSKLQALNMAFASSMAEIAVAEQG |
| | GLSLEAKTNAIANALASAFLETTGFVNQQFVSEIKSLIYMIAQASSNEISGSAAAAGGGSGGGGGSGQGGYGQGASASASAAAA |
| 91 | GGGDGYGQGGYGNQRGVGSYGQGAGAGAAATSAAGGAGSGRGGYGEQGGLGGYGQGAGAGAASTAAGGGDGYGQGGYG |
| | NQGGRGSYGQGSGAGAGAAVAAAAGGAVSGQGGYDGEGGQGGYGQGSGAGAAVAAASGGTGAGQGGYGSQGSQAGYGQ |
| | GAGFRAAAATAAA |
| 92 | GAGAGYGGQVGYGQGAGASAGAAAAGAGAGYGGQAGYGQGAGGSAGAAAAGAGAGRQAGYGQGAGASARAAAAGAGT |
| | GYGQGAGASAGAAAAGAGAGSQVGYGQGAGASSGAAAAAGAGAGYGGQVGYEQGAGASAGAEAAASSAGAGYGGQAGY |
| | GQGAGASAGAAAA |
| 93 | GGAGQGGYGGLGGQGAGQGGLGGQRAGAAAAAAGGAGQGGYGGLGSQGAGRGGYGGVGSGASAASAAASRLSSPEASSRV |
| | SSAVSNLVSSGPTNSAALSSTISNVVSQISASNPGLSGCDVLVQALLEVVSALIQILGSSSIGQVNYGTAGQAAQIVGQSVYQALG |
| 94 | GGYGPGSGQQGPGGAGQQGPGGQGPYGPGSSSAAAVGGYGPSSGLQGPAGQGPYGPGAAASAAAAAGASRLSSPQASSRVSS |
| | AVSSLVSSGPTNSAALTNTISSVVSQISASNPGLSGCDVLIQALLEIVSALVHILGYSSIGQINYDAAAQYASLVGQSVAQALA |
| 95 | GGAGAGQGSYGGQGGYGQGGAGAATATAAAAGGAGSGQGGYGGQGGLGGYGQGAGAGAAAAAAAAAGGAGAGQGGYG |
| | GQGGQGGYGQGAGAGAAAAAAGGAGAGQGGYGGQGGYGQGGGAGAAAAAAAASGGSGSGQGGYGGQGGLGGYGQGAG |
| | AGAGAAASAAAA |
| 96 | GQGGQGGYGRQSQGAGSAAAAAAAAAAAAAAGSGQGGYGGQGQGGYGQSSASASAAASAASTVANSVSRLSSPSAVSRVSS |
| | AVSSLVSNGQVNMAALPNIISNISSSVSASAPGASGCEVIVQALLEVITALVQIVSSSSVGYINPSAVNQITNVVANAMAQVMG |
| 97 | GGAGQGGYGGLGGQGSGAAAAGTGQGGYGSLGGQGAGAAGAAAAAVGGAGQGGYGGVGSAAASAAASRLSSPEASSRVSS |
| | AVSNLVSSGPTNSAALSNTISNVVSQISSSNPGLSGCDVLVQALLEVVSALIHILGSSSIGQVNYGSAGQATQIVGQSVYQALG |
| 98 | GAGAGGAGGYGAGQGYGAGAGAGAAAGAGAGGARGYGARQGYGSGAGAGAGARAGGAGGYGRGAGAGAAAASGAGAG |
| | GYGAGQGYGAGAGAVASAAAGAGSGAGGAGGYGRGAGAVAGAGAGGAGGYGAGAGAAAGVGAGGSGGYGGRQGGYSA |
| | GAGAGAAAAA |
| 99 | GQGGQGGYGGLGQGGYGQGAGSSAAAAAAAAAAAGRGQGGYGQGSGGNAAAAAAAAAAAASGQGGQGGQGGQGQGGYG |
| | QGAGSSAAAAAAAAAAAAAAAGRGQGGYGQGAGGNAAAAAAAAAAAASGQGGQGGQGGQGQGGYGQGAGSSAAAAAAA |
| | AAAAAAA |
| 100 | GGYGPGSGQQGPGQQGPGQQGPGQQGPYGAGASAAAAAAGGYGPGSGQQGPGVRVAAPVASAAASRLSSSAASSRVSSAVSS |
| | LVSSGPTTPAALSNTISSAVSQISASNPGLSGCDVLVQALLEVVSALVHILGSSSVGQINYGASAQYAQMVGQSVTQALV |
| 101 | GAGAGGAGYGRGAGAGAGAAAGAGAGAAAGAGAGAGGYGGQGGYGAGAGAGAAAAAGAGAGGAAGYSRGGRAGAAGA |
| | GAGAAAGAGAGAGGYGGQGGYGAGAGAGAAAAAGAGSGGAGGYGRGAGAGAAAGAGAAAGAGAGAGGYGGQGGYGAG |
| | AGAAAAA |
| 102 | GAGAGRGGYGRGAGAGGYGGQGGYGAGAGAGAAAAAGAGAGGYGDKEIACWSRCRYTVASTTSRLSSAEASSRISSAASTL |
| | VSGGYLNTAALPSVISDLFAQVGASSPGVSDSEVLIQVLLEIVSSLIHILSSSSVGQVDFSSVGSSAAAVGQSMQVVMG |
| 103 | GAGAGAGGAGGYGRGAGAGAGAGAGAAAGQGYGSGAGAGAGASAGGAGSYGRGAGAGAAAASGAGAGGYGAGQGYGA |
| | GAGAVASAAAGAGSGAGGAGGYGRGAVAGSGAGAGAGAGGAGGYGAGAGAGAAAGAVAGGSGGYGGRQGGYSAGAGAG |
| | AAAAA |
| 104 | GPGGYGPVQQGPSGPGSAAGPGGYGPAQQGPARYGPGSAAAAAAAAGSAGYGPGPQASAAASRLASPDSGARVASAVSNLVS |
| | SGPTSSAALSSVISNAVSQIGASNPGLSGCDVLIQALLEIVSACVTILSSSSIGQVNYGAASQFAQVVGQSVLSAFS |
| 105 | GTGGVGGLFLSSGDFGRGGAGAGAGAAAASAAAASSAAAGARGGSGFGVGTGGFGRGGAGAGTGAAAASAAAASAAAAGA |
| | GGDGGLFLSSGDFGRGGAGAGAGAAAASAAAASSAAAGARGGSGFGVGTGGFGRGGAGDGASAAAASAAAASAAAA |
| 106 | GGYGPGAGQQGPGGAGQQGPGGQGPYGPSVAAAASAAGGYGPGAGQQGPVASAAVSRLSSPQASSRVSSAVSSLVSSGPTNPA |
| | ALSNAMSSVVSQVSASNPGLSGCDVLVQALLEIVSALVHILGSSSIGQINYAASSQYAQMVGQSVAQALA |
| 107 | GGAGQGGYGGLGSQGAGRGGYGGQGAGAAAAATGGAGQGGYGGVGSGASAASAAASRLSSPQASSRVSSAVSNLVASGPTN |
| | SAALSSTISNAVSQIGASNPGLSGCDVLIQALLEVVSALIHILGSSSIGQVNYGSAGQATQIVGQSVYQALG |
| 108 | GGAGQGGYGGLGSQGAGRGGYGGQGAGAAVAAIGGVGQGGYGGVGSGASAASAAASRLSSPEASSRVSSAVSNLVSSGPTNS |
| | AALSSTISNVVSQIGASNPGLSGCDVLIQALLEVVSALVHILGSSSIGQVNYGSAGQATQIVGQSVYQALG |
| 109 | GASGGYGGGAGEGAGAAAAAGAGAGGAGGYGGGAGSGAGAVARAGAGGAGGYGSGIGGGYGSGAGAAAGAGAGGAGAY |
| | GGGYGTGAGAGARGADSAGAAAGYGGGVGTGTGSSAGYGRGAGAGAGAGAAAGSGAGAAGGYGGGYGAGAGAGA |
| 110 | GAGSGQGGYGGQGGLGGYGQGAGAGAAAGASGSGSGGAGQGGLGGYGQGAGAGAAAAAAGASGAGQGGFGPYGSSYQSS |
| | TSYSVTSQGAAGGLGGYGQGSGAGAAAAGAAGQGGQGGYGQGAGAGAGAGAGQGGLGGYGQGAGSSAASAAAA |
| 111 | GGAGQGGYGGLGGQGVGRGGLGGQGAGAAAAGGAGQGGYGGVGSGASAASAAASRLSSPQASSRLSSAVSNLVATGPTNSA |
| | ALSSTISNVVSQIGASNPGLSGCDVLIQALLEVVSALIQILGSSSIGQVNYGSAGQATQIVGQSVYQALG |
| 112 | GAGSGGAGGYGRGAGAGAGAAAGAGAGAGSYGGQGGYGAGAGAGAAAAAGAGAGAGGYGRGAGAGAGAGAGAAARAG |
| | AGAGGAGYGGQGGYGAGAGAGAAAAAGAGAGGAGGYGRGAGAGAGAAAGAGAGAGGYGGQSGYGAGAGAAAAA |
| 113 | GASGAGQGQGYGQQGQGGSSAAAAAAAAAAAQGQGQGYGQQGQGYGQQGQGGSSAAAAAAAAAAAAAQGQGQGYGQQG |
| | QGSAAAAAAAAAGASGAGQGQGYGQQGQGGSSAAAAAAAAAAAAAAAQGQGYGQQGQGSAAAAAAAAAAAAA |
| 114 | GGYGPGAGQQGPGSGGQQGPGGQGPYGSGQQGPGGAGQQGPGGQGPYGPGAAAAAAAAAGGYGPGAGQQGPGGAGQQGP |
| | GSQGPGGQGPYGPGAGQQGPGSQGPGSGGQQGPGGQGPYGPSAAAAAAAAAGGYGPGAGQRSQGPGGQGPYGPGAGQQGPG |
| | SQGPGSGGQQGPGGQGPYGPSAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGGQGPYGPSAAAAAAAAGGYGPG |
| | AGQQGPGSGGQQGPGGQGPYGSGQQGPGGAGQQGPGGQGPYGPGAAAAAAAAAGGYGPGAGQQGPGGAGQQGPGSQGPGG |
| | QGPYGPGAGQQGPGSQGPGSGGQQGPGGQGPYGPSAAAAAAAAAGGYGPGAGQRSQGPGGQGPYGPGAGQQGPGSQGPGSG |
| | GQQGPGGQGPYGPSAAAAAAAAGGYGPGAGQQGPGSQGPGSGGQQGPGGQGPYGPGAAAAAAAVGGYGPGAGQQGPGSQG |
| | PGSGGQQGPGGQGPYGPSAAAAAAAAGGYGPGAGQQGPGSQGPGSGGQQGPGGQGPYGPSAAAAAAAAGGYGPGAGQQGP |
| | GSGGQQGPGGQGPYGSGQQGPGGAGQQGPGGQGPYGPGAAAAAAAAAGGYGPGAGQQGPGGAGQQGPGSQGPGGQGPYGP |
| | GAGQQGPGSQGPGSGGQQGPGGQGPYGPSAAAAAAAAAGGYGPGAGQRSQGPGGQGPYGPGAGQQGPGSQGPGSGGQQGPG |
| | GQGPYGPSAAAAAAAAGGYGPGAGQQGPGSQGPGSGGQQGPGGQGPYGPGAAAAAAAVGGYGPGAGQQGPGSQGPGSGGQ |
| | QGPGGQGPYGPSAAAAAAAAGGYGPGAGQQGPGSQGPGSGGQQGPGGQGPYGPSAAAAAAAA |

In some embodiments, the silk or silk or silk-like proteins comprise one or more repeat units comprising SEQ ID NO: 17. This repeat unit contains 6 quasi-repeat units. The quasi-repeat unit can be concatenated 2, 3, 4, 5, 6, 7, 8, 9, 10, 11, 12, 13, 14, 15, 16, 17, 18, 19, or 20 times to form polypeptide molecules from about 50 kDal to about 1,000 kDal. This repeat unit also contains poly-alanine regions related to nano-crystalline regions, and glycine-rich regions related to beta-turn containing less-crystalline regions.

Non-limiting examples of additional suitable silk or silk or silk-like proteins are provided, for example, in International Patent Publication WO/2016/201369, published Dec. 15, 2016; U.S. patent application 62/394,683, filed Sep. 14, 2016; U.S. patent application Ser. No. 15/705,185, filed Sep. 14, 2017, U.S. publication US20160222174, published Aug. 4, 2016; International Patent Publication WO2016/149414, published Mar. 16, 2016; International Patent Publication WO 2014/066374, published Jan. 5, 2014, and International Patent Publication WO 2015/042164, published Mar. 26, 2015, each of which are hereby incorporated by reference in its entirety.

Typically, operable linkage of proteins with secretion signals requires removal of start codons of the polynucleotide sequences encoding the proteins.

### Other Components

In some embodiments, the polynucleotide sequences comprised in the expression constructs further encode tag peptides or polypeptides operably linked to the C-termini of the proteins. Such tag peptides or polypeptides can aid in purification of the recombinant proteins. Non-limiting examples of tag peptides or polypeptides include affinity tags (i.e., peptides or polypeptides that bind to certain agents or matrices), solubilization tags (i.e., peptides or polypeptides that assist in proper folding of proteins and prevent precipitation), chromatography tags (i.e., peptides or polypeptides that alter the chromatographic properties of a protein to afford different resolution across a particular separation techniques), epitope tags (i.e., peptides or polypeptides that are bound by antibodies), fluorescence tags, chromogenic tags, enzyme substrate tags (i.e., peptides or polypeptides that are the substrates for specific enzymatic reactions), chemical substrate tags (i.e., peptides or polypeptides that are the substrates for specific chemical modifications), or combinations thereof. Non-limiting examples of suitable affinity tags include maltose binding protein (MBP), glutathione-S-transferase (GST), poly (His) tag, SBP-tag, Strep-tag, and calmodulin-tag. Non-limiting examples of suitable solubility tags include thioredoxin (TRX), poly (NANP), MBP, and GST. Non-limiting examples of chromatography tags include polyanionic amino acids (e.g., FLAG-tag) and polyglutamate tag. Non-limiting examples of epitope tags include V5-tag, VSV-tag, Myc-tag, HA-tag, E-tag, NE-tag, Ha-tag, Myc-tag, and FLAG-tag. Non-limiting examples of fluorescence tags include green fluorescent protein (GFP), blue fluorescent protein (BFP), cyan fluorescent protein (CFP), yellow fluorescent protein (YFP), orange fluorescent protein (OFP), red fluorescent protein (RFP), and derivatives thereof. Non-limiting examples of enzyme substrate tags include peptides or polypeptides comprising a lysine within a sequence suitable for biotinilation (e.g., AviTag, Biotin Carboxyl Carrier Protein [BCCP]). Non-limiting examples of chemical substrate tags include substrates suitable for reaction with FIASH-EDT2. The fusion of the C-terminal tag peptide or polypeptide to the recombinant proteins can be cleavable (e.g., by TEV protease, thrombin, factor Xa, or enteropeptidase) or non-cleavable.

In some embodiments, the polynucleotide sequences comprised in the expression constructs further encode linker peptides operably linked between the proteins and the recombinant secretion signals. The linker peptides can have various sizes. In some such embodiments, the polynucleotide sequences that encode the linker peptides comprise restriction enzyme sites to permit replacement or addition of other polynucleotide sequences.

The expression constructs may further comprise promoters that are operably linked to the polynucleotide sequences encoding the proteins that are operably linked to the recombinant secretion signals such that they drive the transcription of the polynucleotide sequences. The promotors may be constitutive promoters or inducible promoters. Induction may, for example, occur via glucose repression, galactose induction, sucrose induction, phosphate repression, thiamine repression, or methanol induction. Suitable promoters are promoters that mediate expression of proteins in the recombinant host cells provided herein. Non-limiting examples of suitable promoters include the alcohol oxidase (AOX1) promoter of Pichia pastoris (pAOX1), glyceraldehyde-3-phosphate dehydrogenase (GAP) promoter of Pichia pastoris (pGAP), YPT1 promoter, 3-phosphoglycerate kinase 1 (PGK1) promoter of Saccharomyces cerevisae (pPKG1), SSA4 promoter, HSP82 promoter, GPM1 promoter, KAR2 promoter, triose phosphate isomerase 1 (TPI1) promoter of Pichia pastoris (pTPI1), enolase 1 (ENO1) promoter of Pichia pastoris (pENO1), PET9 promoter, PEX8 (PER3) promoter, AOX2 promoter, AOD promoter, THI11 promoter, DAS promoter, FLD1 promoter, PHO89 promoter, CUP1 promoter, GTH1 promoter, ICL1 promoter, TEF1 promoter, LAC4-PBI promoter, T7 promoter, TAC promoter, GCW14 promoter, GAL1 promoter, APL promoter, APR promoter, beta-lactamase promoter, spa promoter, CYC1 promoter, TDH3 promoter, GPD promoter, translation initiation factor 1 (TEF1) promoter of Saccharomyces cerevisiae, ENO2 promoter, PGL1 promoter, GAP promoter, SUC2 promoter, ADH1 promoter, ADH2 promoter, HXT7 promoter, PHO5 promoter, and CLB1 promoter. Additional promoters that can be used are known in the art.

The expression constructs may further comprise terminators that are operably linked to the polynucleotide sequences encoding the proteins that are operably linked to the recombinant secretion signals such that they effect termination of transcription of the polynucleotide sequences. Suitable terminators are terminators that terminate transcription in the recombinant host cells provided herein. Non-limiting examples of suitable terminators include the AOX1 terminator of Pichia pastoris (tAOX1), PGK1 terminator, and TPS1 terminator. Additional terminators are known in the art.

### Recombinant Vectors

The recombinant vectors provided herein comprise expression constructs provided herein. In some embodiments, the recombinant vectors comprise multiple expression constructs (e.g., 2, 3, 4, 5, etc.). In some such embodiments, the expression constructs are identical. In other such embodiments, at least 2 of the expression constructs are not identical. In embodiments in which at least 2 of the expression constructs are not identical, the at least 2 expression constructs may differ from each other in the proteins, recombinant secretion signals, promoters, terminators, and/or other components they encode.

The recombinant vectors may further comprise elements suitable for propagation of the recombinant vectors in recombinant host cells. Non-limiting examples of such other elements include origins of replication and selection markers (e.g., antibiotic resistance genes, auxotrophic markers). Origins of replication and selection markers are known in the art. In various embodiments, the origins of replications are bacterial or yeast origins of replication. In some embodiments, the origins of replication are Pichia autonomously replicating sequences (PARS). In some embodiments, the selection markers are drug resistant markers. A drug resistant maker enables cells to detoxify an exogenously added drug that would otherwise kill the cell. Illustrative examples of drug resistant markers include but are not limited to those for resistance to antibiotics such as ampicillin, tetracycline, kanamycin, bleomycin, streptomycin, hygromycin, neomycin, Zeocin™, and the like. In other embodiments, the selection markers are auxotrophic markers. An auxotrophic marker allows cells to synthesize an essential component (usually an amino acid) while grown in media that lacks that essential component. Selectable auxotrophic markers include, for example, hisD, which allows growth in histidine-free media in the presence of histidinol. Other selection markers include a bleomycin-resistance gene, a metallothionein gene, a hygromycin B-phosphotransferase gene, the AURI gene, an adenosine deaminase gene, an aminoglycoside phosphotransferase gene, a dihydrofolate reductase gene, a thymidine kinase gene, and a xanthine-guanine phosphoribosyltransferase gene.

The recombinant vectors may further comprise targeting sequences that can direct integration of the expression constructs to specific locations in the genome of host cells. Non-limiting examples of such targeting sequences are polynucleotide sequences that are homologous to polynucleotide sequences comprised in the genome of host cells. In some embodiments, the targeting sequences are homologous to repetitive elements in the genome of host cells. In some embodiments, the targeting sequences are homologous to transposable elements in the genome of host cells.

### Recombinant Host Cells

The recombinant host cells provided herein are cells that comprise expression constructs provided herein. The recombinant host cells can be of mammalian, plant, algae, fungi, or microbe origin.

Non-limiting examples of suitable fungi include methylotrophic yeast, filamentous yeast, Arxula adeninivorans, Aspergillus niger, Aspergillus niger var. awamori, Aspergillus oryzae, Candida etchellsii, Candida guilliermondii, Candida humilis, Candida lipolytica, Candida pseudotropicalis, Candida utilis, Candida versatilis, Debaryomyces hansenii, Endothia parasitica, Eremothecium ashbyii, Fusarium moniliforme, Hansenula polymorpha, Kluyveromyces lactis, Kluyveromyces marxianus, Kluyveromyces thermotolerans, Morteirella vinaceae var. raffinoseutilizer, Mucor miehei, Mucor miehei var. Cooney et Emerson, Mucor pusillus Lindt, Penicillium roquefortii, Pichia methanolica, Pichia (Komagataella) pastoris, Pichia (Scheffersomyces) stipitis, Rhizopus niveus, Rhodotorula sp., Saccharomyces bayanus, Saccharomyces beticus, Saccharomyces cerevisiae, Saccharomyces chevalieri, Saccharomyces diastaticus, Saccharomyces ellipsoideus, Saccharomyces exiguus, Saccharomyces florentinus, Saccharomyces fragilis, Saccharomyces pastorianus, Saccharomyces pombe, Saccharomyces sake, Saccharomyces uvarum, Sporidiobolus johnsonii, Sporidiobolus salmonicolor, Sporobolomyces roseus, Trichoderma reesi, Xanthophyllomyces dendrorhous, Yarrowia lipolytica, Zygosaccharomyces rouxii, and derivatives and crosses thereof.

Non-limiting examples of suitable microbes include Acetobacter suboxydans, Acetobacter xylinum, Actinoplane missouriensis, Arthrospira platensis, Arthrospira maxima, Bacillus cereus, Bacillus coagulans, Bacillus licheniformis, Bacillus stearothermophilus, Bacillus subtilis, Escherichia coli, Lactobacillus acidophilus, Lactobacillus bulgaricus, Lactobacillus reuteri, Lactococcus lactis, Lactococcus lactis Lancefield Group N, Leuconostoc citrovorum, Leuconostoc dextranicum, Leuconostoc mesenteroides strain NRRL B-512 (F), Micrococcus lysodeikticus, Spirulina, Streptococcus cremoris, Streptococcus lactis, Streptococcus lactis subspecies diacetylactis, Streptococcus thermophilus, Streptomyces chattanoogensis, Streptomyces griseus, Streptomyces natalensis, Streptomyces olivaceus, Streptomyces olivochromogenes, Streptomyces rubiginosus, Xanthomonas campestris, and derivatives and crosses thereof.

Additional strains that can be used as recombinant host cells are known in the art. It should be understood that the term “recombinant host cell” is intended to refer not only to the particular subject cell but to the progeny of such a cell. Because certain modifications may occur in succeeding generations due to either mutation or environmental influences, such progeny may not, in fact, be identical to the parent cell, but is still included within the scope of the term “recombinant host cell” as used herein.

In some embodiments, the expression constructs are stably integrated within the genome (e.g., a chromosome) of the recombinant host cells, e.g., via homologous recombination or targeted integration. Non-limiting examples of suitable sites for genomic integration include the Ty1 loci in the Saccharomyces cerevisiae genome, the rDNA and HSP82 loci in the Pichia pastoris genome, and transposable elements that have copies scattered throughout the genome of the recombinant host cells. In other embodiments, the expression constructs are not stably integrated within the genome of the recombinant host cells but rather are maintained extrachromosomally (e.g., on a plasmid).

Production of recombinant proteins can be influenced by the number of copies of the expression constructs provided herein that are comprised in the recombinant host cells and/or the rate of transcription of the polynucleotide sequences comprised in the expression constructs. In some embodiments, the recombinant host cells comprise a single expression construct. In other embodiments, the recombinant host cells comprise 2 or more (e.g., 3, 4, 5, or more) expression constructs. In some embodiments, the recombinant host cells comprise expression constructs that comprise polynucleotide sequences that are operably linked to strong promoters. Non-limiting examples of strong promoters include the pGCW14 promoter of Pichia pastoris. In some embodiments, the recombinant host cells comprise expression constructs that comprise polynucleotide sequences that are operably linked to medium promoters. Non-limiting examples of such medium promoters include the pGAP promoter of Pichia pastoris. In some embodiments, the recombinant host cells comprise expression constructs that comprise polynucleotide sequences that are operably linked to weak promoters.

The recombinant secretion signals provided herein provide high secreted yields of recombinant proteins. Accordingly, in various embodiments, the recombinant host cells produce secreted yields of the protein encoded by the polynucleotide sequences comprised in the expression constructs of at least 1%, 5%, 10%, 20%, or 30%; from 1% to 100%, 90%, 80%, 70%, 60%, 50%, 40%, 30%, 20%, or 10%; from 10% to 100%, 90%, 80%, 70%, 60%, 50%, 40%, 30%, or 20%; from 20% to 100%, 90%, 80%, 70%, 60%, 50%, 40%, or 30%; from 30% to 100%, 90%, 80%, 70%, 60%, 50%, or 40%; from 40% to 100%, 90%, 80%, 70%, 60%, or 50%; from 50% to 100%, 90%, 80%, 70%, or 60%; from 60% to 100%, 90%, 80%, or 70%; from 70% to 100%, 90%, or 80%; from 80% to 100%, or 90%; or from 90% to 100% by weight of total yields of the protein. The identities of recombinant proteins produced can be confirmed by HPLC quantification, Western blot analysis, polyacrylamide gel electrophoresis, and 2-dimensional mass spectroscopy (2D-MS/MS) sequence identification.

### Fermentations

The fermentations provided herein comprise recombinant host cells provided herein and culture media suitable for growing the recombinant host cells.

The fermentations are obtained by culturing the recombinant host cells in culture media that provide nutrients needed by the recombinant host cells for cell survival and/or growth. Such culture media typically contain an excess carbon source. Non-limiting examples of suitable carbon sources include monosaccharides, disaccharides, polysaccharides, alcohols, and combinations thereof. Non-limiting examples of suitable monosaccharides include glucose, galactose, mannose, fructose, ribose, xylose, arabinose, ribose, and combinations thereof. Non-limiting examples of suitable disaccharides include sucrose, lactose, maltose, tehalose, cellobiose, and combinations thereof. Non-limiting examples of suitable polysaccharides include raffinose, starch, glycogen, glycan, cellulose, chitin, and combinations thereof. Non-limiting examples of suitable alcohols include methanol and glycol.

The recombinant secretion signals provided herein provide high secreted yields of recombinant proteins. Accordingly, in various embodiments, the fermentations provided herein comprise at least 1%, 5%, 10%, 20%, or 30%; from 1% to 100%, 90%, 80%, 70%, 60%, 50%, 40%, 30%, 20%, or 10%; from 10% to 100%, 90%, 80%, 70%, 60%, 50%, 40%, 30%, or 20%; from 20% to 100%, 90%, 80%, 70%, 60%, 50%, 40%, or 30%; from 30% to 100%, 90%, 80%, 70%, 60%, 50%, or 40%; from 40% to 100%, 90%, 80%, 70%, 60%, or 50%; from 50% to 100%, 90%, 80%, 70%, or 60%; from 60% to 100%, 90%, 80%, or 70%; from 70% to 100%, 90%, or 80%; from 80% to 100%, or 90%; or from 90% to 100% by weight of total yields of the recombinant proteins as secreted recombinant proteins. In some embodiments, the culture media of the fermentations comprise at least 0.1 g/L, at least 0.5 g/L, at least 1 g/L, at least 2 g/L, at least 5 g/L, at least 7 g/L, at least 10 g/L, at least 15 g/L, or at least 20 g/L; from 0.1 g/L to 30 g/L, to 25 g/L, to 20 g/L, to 15 g/L, to 10 g/L, to 7 g/L, to 5 g/L, to 2 g/L, to 1 g/L, or to 0.5 g/L; from 0.5 g/L to 30 g/L, to 25 g/L, to 20 g/L, to 15 g/L, to 10 g/L, to 7 g/L, to 5 g/L, to 2 g/L, or to 1 g/L; from 1 g/L to 30 g/L, to 25 g/L, to 20 g/L, to 15 g/L, to 10 g/L, to 7 g/L, to 5 g/L, or to 2 g/L; from 2 g/L to 30 g/L, to 25 g/L, to 20 g/L, to 15 g/L, to 10 g/L, to 7 g/L, or to 5 g/L; from 5 g/L to 30 g/L, to 25 g/L, to 20 g/L, to 15 g/L, to 10 g/L, or to 7 g/L; from 7 g/L to 30 g/L, to 25 g/L, to 20 g/L, to 15 g/L, or to 10 g/L; from 10 g/L to 30 g/L, to 25 g/L, to 20 g/L, or to 15 g/L; from 15 g/L to 30 g/L, to 25 g/L, or to 20 g/L; from 20 g/L to 30 g/L, or to 25 g/L; or from 25 g/L to 30 g/L of the recombinant proteins produced by the recombinant host cells.

### Methods of Producing High Secreted Yields of Recombinant Proteins

Provided herein are methods for producing high secreted yields of recombinant proteins. The methods are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y., 1989; Ausubel et al. Current Protocols in Molecular Biology, Greene Publishing Associates, 1992, and Supplements to 2002); Harlow and Lane, Antibodies: A Laboratory Manual, Cold Spring Harbor Laboratory Press, Cold Spring Harbor, N.Y., 1990; Taylor and Drickamer, Introduction to Glycobiology, Oxford Univ. Press, 2003; Worthington Enzyme Manual, Worthington Biochemical Corp., Freehold, N.J.; Handbook of Biochemistry: Section A Proteins, Vol I, CRC Press, 1976; Handbook of Biochemistry: Section A Proteins, Vol II, CRC Press, 1976; Essentials of Glycobiology, Cold Spring Harbor Laboratory Press, 1999.

The methods provided herein comprise the step of culturing recombinant host cells provided herein in culture media under conditions suitable for obtaining the fermentations provided herein (step 1003 in FIG. 1). Suitable culture media for use in these methods are known in the art, as are suitable culture conditions. Details of culturing yeast host cells, for example, are described in Idiris et al. (2010) Appl. Microbiol. Biotechnol. 86:403-417; Zhang et al. (2000) Biotechnol. Bioprocess. Eng. 5:275-287; Zhu (2012) Biotechnol. Adv. 30:1158-1170; and Li et al. (2010) MAbs 2:466-477.

In some embodiments, the methods further comprise the step of constructing expression constructs and/or recombinant vectors provided herein (step 1001 in FIG. 1). Methods for constructing expression construct and recombinant vectors are known in the art. In some embodiments, the expression constructs and/or recombinant vectors are synthetically generated. In other embodiments, the expression constructs and/or recombinant vectors are isolated or PCR amplified by standard procedures from organisms, cells, tissues, or plasmid constructs. In some embodiments, the expression constructs and/or recombinant vectors are codon-optimized for expression in particular host cells.

In some embodiments, the methods comprise the step of balancing expression of the recombinant proteins (e.g., by increasing or reducing the number of polynucleotide sequences and/or the strengths of the promoters that are operably linked to the polynucleotide sequences) and efficiency of secretion of the recombinant proteins (e.g., by choosing specific recombinant secretion signals).

In some embodiments, the methods further comprise the step of transforming cells with expression constructs or recombinant vectors provided herein to obtain recombinant host cells provided herein (step 1002 in FIG. 1). For such transformations, the recombinant vectors can be circularized or be linear. Methods for transforming cells are well-known in the art. Non-limiting examples of such methods include calcium phosphate transfection, dendrimer transfection, liposome transfection (e.g., cationic liposome transfection), cationic polymer transfection, electroporation, cell squeezing, sonoporation, optical transfection, protoplast fusion, impalefection, hyrodynamic delivery, gene gun, magnetofection, spheroblast generation, polyethylene glycol (PEP) treatment, and viral transduction. One skilled in the art is able to select one or more suitable methods for transforming cells with expression constructs or recombinant vectors provided herein based on the knowledge in the art that certain techniques for introducing vectors work better for certain types of cells. Recombinant host cell transformants comprising expression constructs or recombinant vectors provided herein can be readily identified, e.g., by virtue of expressing drug resistance or auxotrophic markers encoded by the recombinant vectors that permit selection for or against growth of cells, or by other means (e.g., detection of light emitting peptide comprised in the expression constructs or recombinant vectors, molecular analysis of individual recombinant host cell colonies [e.g., by restriction enzyme mapping, PCR amplification, or sequence analysis of isolated extrachromosomal vectors or chromosomal integration sites]).

In some embodiments, the methods further comprise the step of extracting the secreted recombinant proteins from fermentations provided herein (step 1004 in FIG. 1). Extraction can occur by a variety of methods known in the art for purifying secreted proteins. Common steps in such methods include centrifugation at speeds that cause the pelleting of cells and removal of cell pellets comprising the recombinant host cells and cell debris, followed by precipitation of the recombinant proteins using precipitants (e.g., ammonium sulfate at 5-60% saturation; followed by centrifugation) or affinity separation (e.g., by immunological interaction with antibodies that bind specifically to the recombinant proteins or their C-terminal tags [e.g., FLAG, hemagglutinin], or via binding to nickel columns for isolation of polypeptides tagged with 6 to 8 histidine residues (SEQ ID NO: 154)). The suspended recombinant proteins can be dialyzed to remove the dissolved salts. Additionally, the dialyzed recombinant proteins can be heated to denature other proteins, and the denatured proteins can be removed by centrifugation.

### Examples

### Example 1: Generation of Pichia pastoris Recombinant Host Cells that Produce High Secreted Yields of Silk-Like Proteins

Pichia pastoris (Komagataella phaffii) recombinant host cells that secrete a silk-like protein were generated by transforming a HIS+derivative of GS115 (NRRL Y15851) with various recombinant vectors.

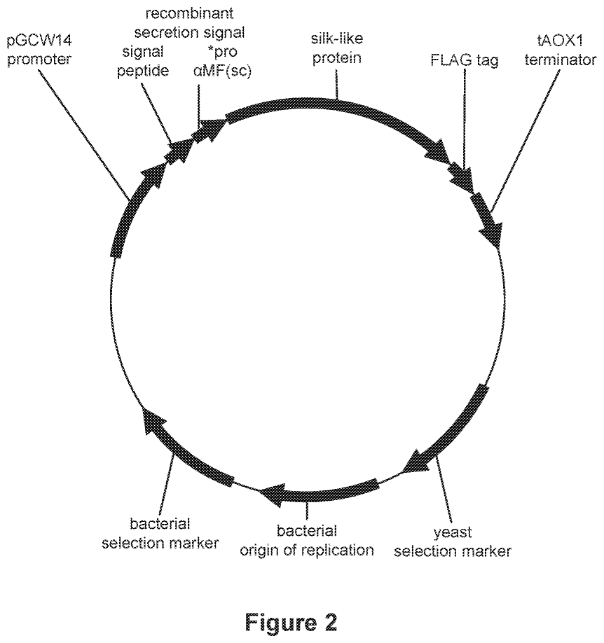

The recombinant vectors (see FIG. 2) comprised an expression construct that comprised a polynucleotide sequence encoding the silk-like protein (SEQ ID NO: 114) operably linked to various N-terminal recombinant secretion signals. The recombinant secretion signals consisted of an N-terminal signal peptide operably linked to *pro-αMF (sc) (SEQ ID NO:2) or to pro-EPX1 (pp) (SEQ ID NO: 144). The silk-like protein was further operably linked to a C-terminal FLAG-tag. Each of the polynucleotide sequences was flanked by a promoter (pGCW14) and a terminator (tAOX1 pA signal). The recombinant vectors further comprised a targeting region that directed integration of the expression construct to the region immediately 3′ of the ICL1, HSP82, or THI13 loci in the Pichia pastoris genome, dominant resistance markers for selection of bacterial and yeast transformants, and a bacterial origin of replication.

The recombinant vectors were transformed into the Pichia pastoris host cells via electroporation to generate recombinant host strains. Transformants were plated on YPD agar plates supplemented with antibiotics, and incubated for 48 hours at 30° C.

Clones from each final transformation were inoculated into 400 μL of Buffered Glycerol-complex Medium (BMGY) in 96-well blocks, and incubated for 48 hours at 30° C. with agitation at 1,000 rpm. Following the 48-hour incubation, 4 μL of each culture was used to inoculate 400 μL of minimal media in 96-well blocks, which were then incubated for 48 hours at 30° C.

Guanidine thiocyanate was added to a final concentration of 2.5 M to the cell cultures to extract the recombinant protein for measurement by ELISA. After a 5 min incubation, solutions were centrifuged and the supernatant was sampled.

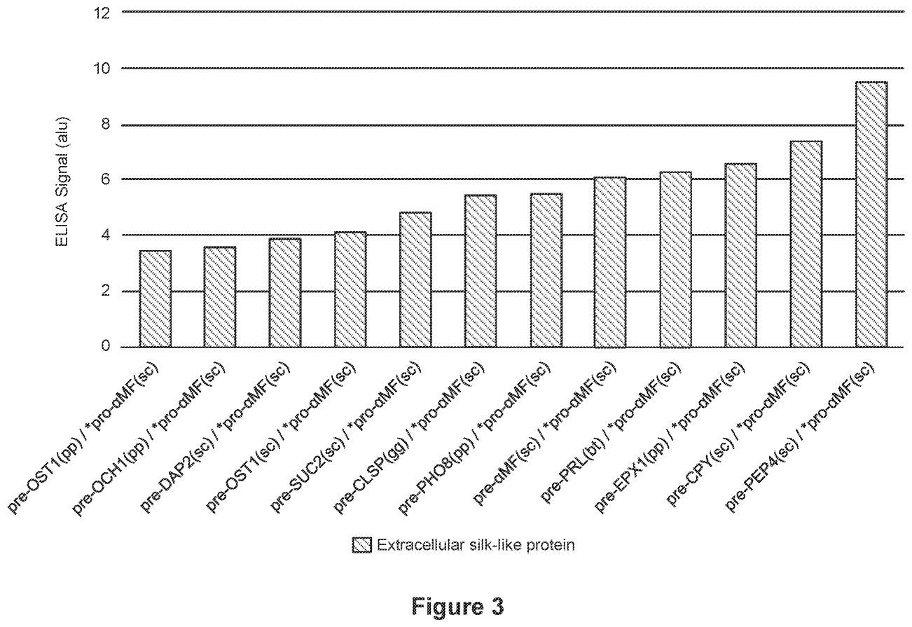

As shown in FIG. 3, a number of recombinant secretion signals produced higher secreted yields of the silk-like protein than the pre-OST1 (sc)/*pro-αMF (sc) recombinant secretion signal and/or the pre-αMF (sc)/*pro-αMF (sc) secretion signal, whereas others produced lower secreted yields.

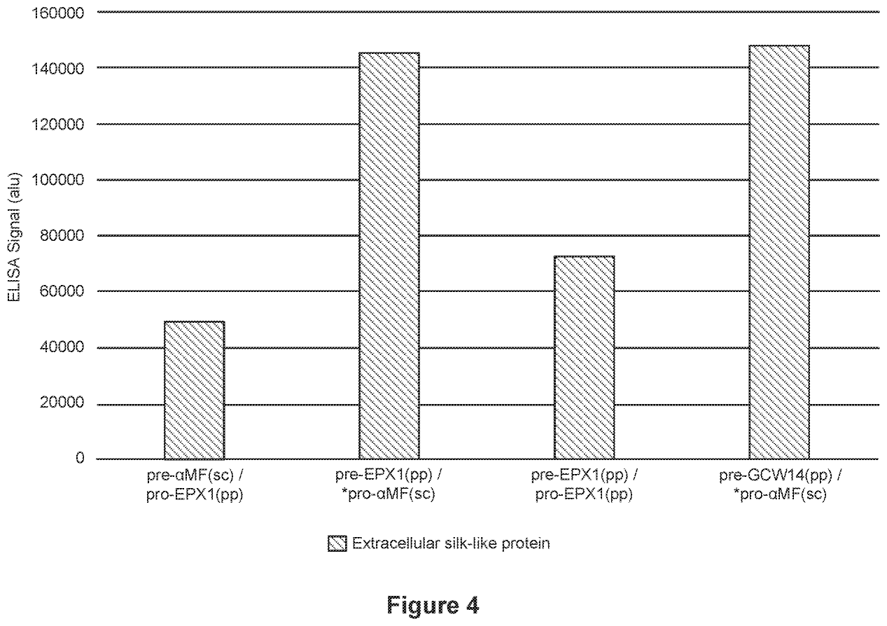

As shown in FIG. 4, secreted yields of the silk-like protein were significantly higher when the recombinant secretion signals comprised pro-αMF (sc) rather than pro-EPX1 (pp). As further shown in FIG. 4, slightly higher secreted yields were obtained with the recombinant secretion signal pre-GCW14 (pp)/*pro-αMF (sc) than with the recombinant secretion signal pre-EPX1 (pp)/*pro-αMF (sc).

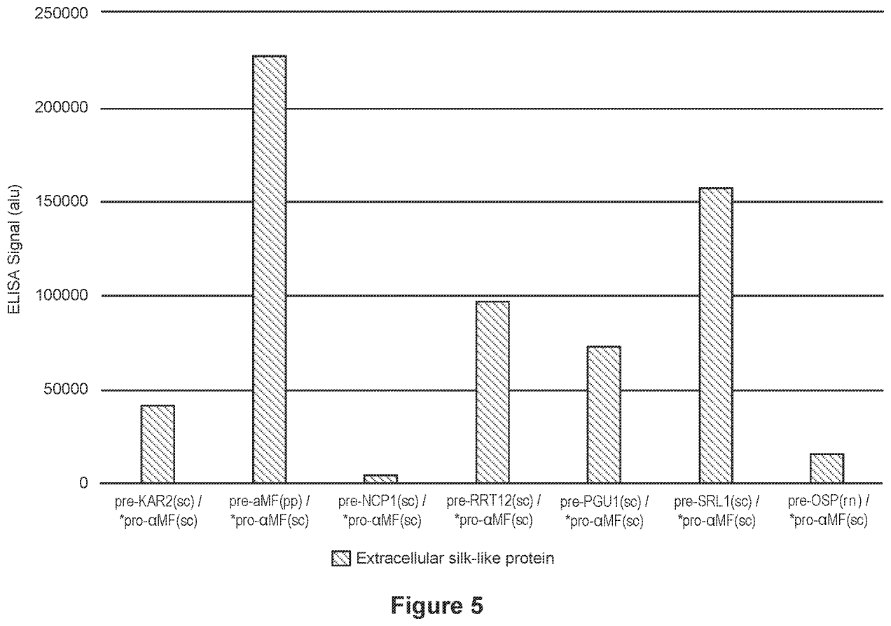

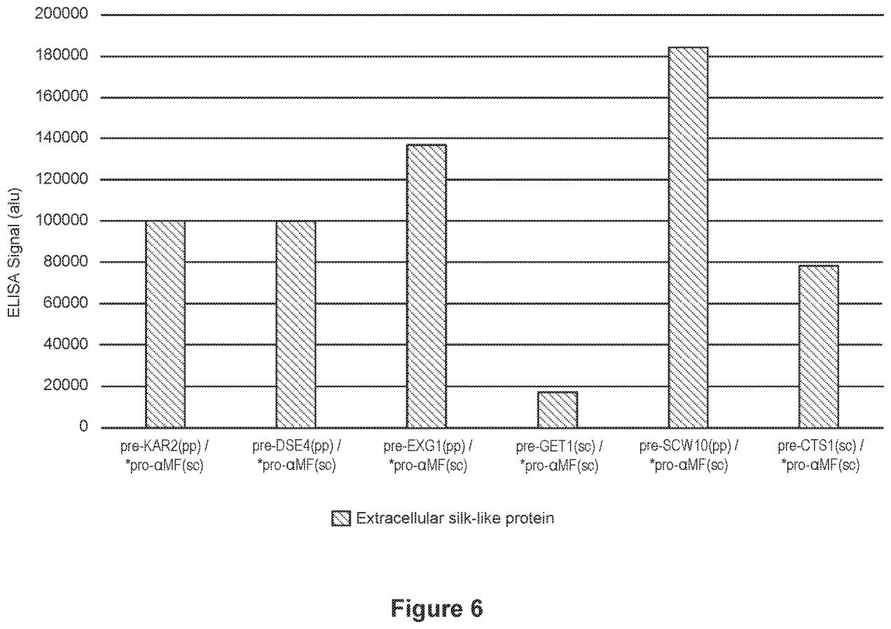

FIGS. 5 and 6 show additional recombinant secretion signals provided herein that achieve secreted yields of the silk-like protein.

### Example 2: Generation of Pichia pastoris Recombinant Host Cells that Produce High Secreted Yields of Alpha-Amylase or Green Fluorescent Protein

Pichia pastoris (Komagataella phaffii) recombinant host cells that secrete either an alpha-amylase or green fluorescent protein were generated by transforming a HIS+derivative of GS115 (NRRL Y15851) with various recombinant vectors.

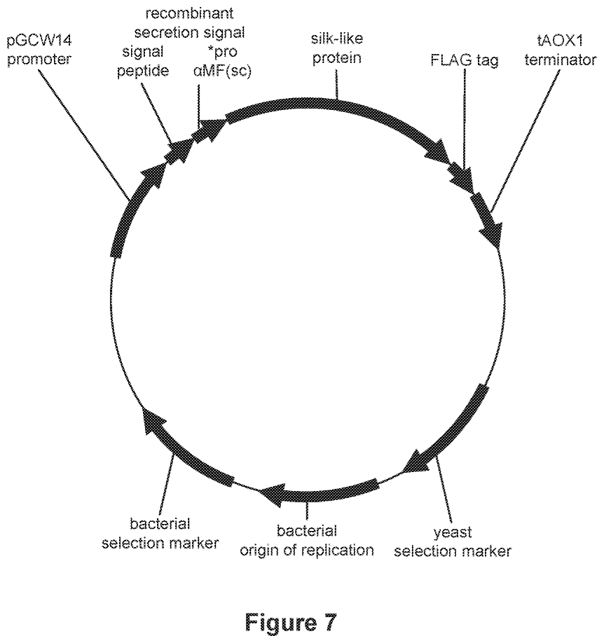

The recombinant vectors (see FIG. 7) comprised an expression construct that comprised a polynucleotide sequence encoding either alpha-amylase (SEQ ID NO: 145) or green fluorescent protein (SEQ ID NO: 146) operably linked to various N-terminal recombinant secretion signals. The recombinant secretion signals consisted of an N-terminal signal peptide operably linked to *pro-αMF (sc) (SEQ ID NO:2). The alpha-amylase or green fluorescent protein was further operably linked to a C-terminal FLAG-tag. Each of the polynucleotide sequences was flanked by a promoter (pGCW14) and a terminator (tAOX1 pA signal). The recombinant vectors further comprised a targeting region that directed integration of the expression construct to the region immediately 3′ of the THI4 locus in the Pichia pastoris genome, dominant resistance markers for selection of bacterial and yeast transformants, and a bacterial origin of replication.

The recombinant vectors were transformed into the Pichia pastoris host cells via electroporation to generate recombinant host strains. Transformants were plated on YPD agar plates supplemented with antibiotics, and incubated for 48-96 hours at 30° C.

Clones from each final transformation were inoculated into 400 μL of Buffered Glycerol-complex Medium (BMGY) in 96-well blocks, and incubated for 48 hours at 30° C. with agitation at 1,000 rpm. Following the 48-hour incubation, 4 μL of each culture was used to inoculate 400 μL of minimal media in 96-well blocks, which were then incubated for 48 hours at 30° C.

Guanidine thiocyanate was added to a final concentration of 2.5 M to the cell cultures to extract the recombinant protein for measurement by ELISA. After a 5 min incubation, solutions were centrifuged and the supernatant was sampled.

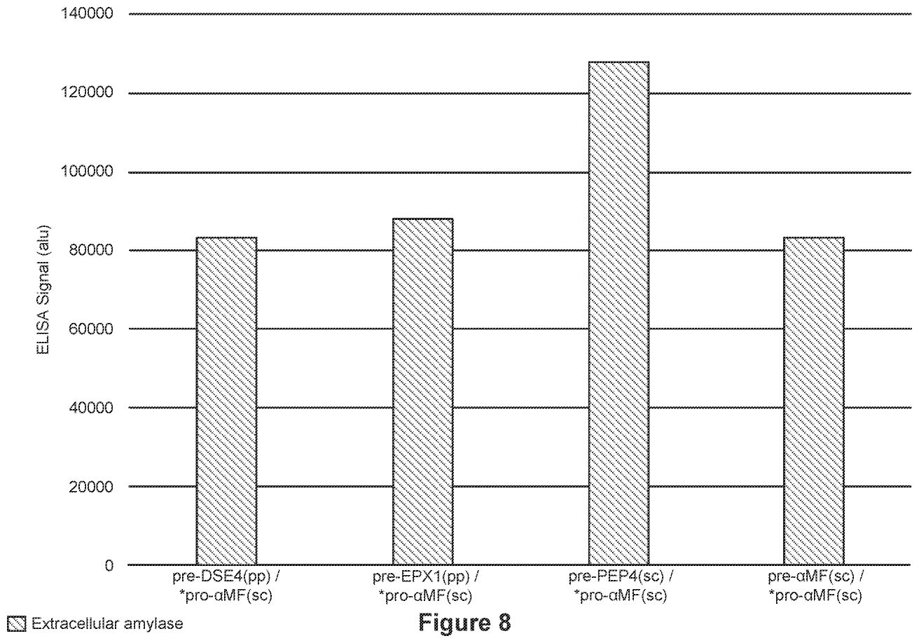

As shown in FIG. 8, the pre-EPX1 (pp)/*pro-αMF (sc) and the pre-PEP4 (sc)/*pro-αMF (sc) recombinant secretion signals produced higher secreted yields of amylase than the pre-αMF (sc)/*pro-αMF (sc) recombinant secretion signal while the pre-DSE4 (pp)/*pro-αMF (sc) secretion signal produced roughly the same amount of secreted amylase.

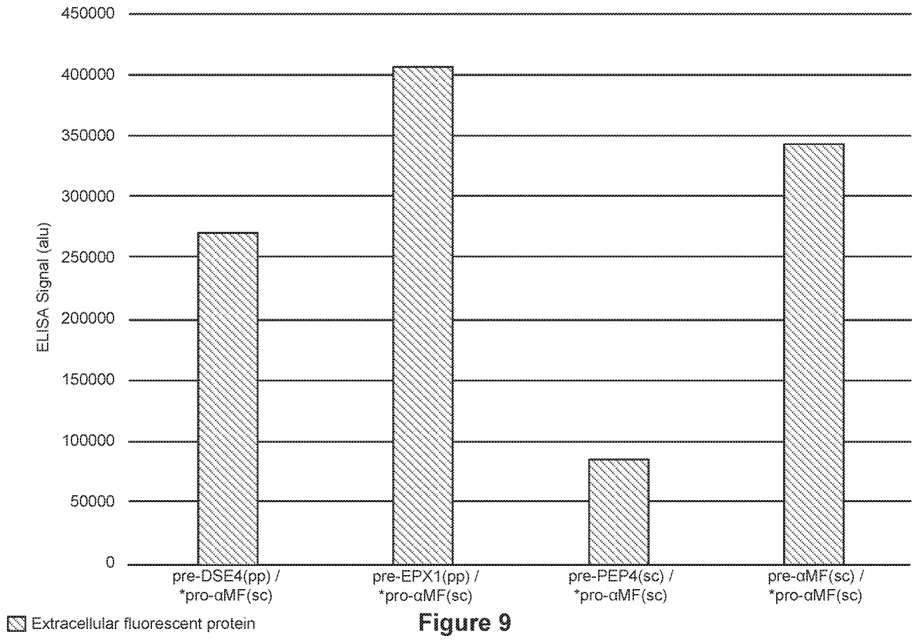

As shown in FIG. 9, the pre-EPX1 (pp)/*pro-αMF (sc) recombinant secretion signal produced higher secreted yields of green fluorescent protein than the pre-αMF (sc)/*pro-αMF (sc) recombinant secretion signal, while the pre-PEP4 (sc)/*pro-αMF (sc) and the pre-DSE4 (pp)/*pro-αMF (sc) secretion signals produced less secreted fluorescent protein.

The foregoing description of the embodiments of the disclosure has been presented for the purpose of illustration; it is not intended to be exhaustive or to limit the scope of the invention.

In the examples, efforts have been made to ensure accuracy with respect to numbers used (e.g., amounts, temperatures, etc.), but some experimental error and deviation should, of course, be allowed for. The reagents employed in the examples are generally commercially available or can be prepared using commercially available instrumentation, methods, or reagents known in the art. The examples are not intended to provide an exhaustive description of the many different embodiments of the invention. Those of ordinary skill in the art will realize readily that many changes and modifications can be made to the embodiments presented in the examples without departing from the spirit or scope of the appended claims.

TABLE 4
Additional Sequences

| SEQ | | |
|---|---|---|
| ID | | |
| NO | Name | Amino Acid Sequence |
| 1 | pro- | APVNTTTEDETAQIPAEAVIGYLDLEGDFDVAVLPFSNSTNNGLLFINTTIA |
| | αMF(sc) | SIAAKEEGVSLDKREAEA |
| 2 | *pro- | APVNTTTEDETAQIPAEAVIGYSDLEGDFDVAVLPFSNSTNNGLLFINTTIA |
| | αMF(sc) | SIAAKEEGVSLEKREAEA |
| 115 | pre- | MRFPSIFTAVLFAASSALAAPVNTTTEDETAQIPAEAVIGYLDLEGDFDVA |
| | αMF(sc)/ | VLPFSNSTNNGLLFINTTIASIAAKEEGVSLDKREAEA |
| | pro- | |
| | αMF(sc) | |
| 144 | pro- | APVAPAEEAANHLHKRAYYTDTTKTHTFTEVVTVYRT |
| | EPX1(pp) | |
| 145 | alpha- | ANLNGTLMQYFEWYMPNDGQHWKRLQNDSAYLAEHGITAVWIPPAYK |
| | amylase | GTS QDDVGYGAYDLYDLGEFHQKGTVRTKYGTKGELQSAINSLHSRDIN |
| | | VYGDVVINHKGGADATEDVTAVEVDPADRNRVTSGEQRIKAWTHFQFP |
| | | GRGSTYSDFKWHWYHFDGTDWDESRKLNRIYKFQGKAWDWEVSNVNG |
| | | NYDYLMYADIDYDHPDATAEIKRWGTWYANELQLDGFRLDAVKHIKFSF |
| | | LRDWVNHVREKTGKEMFTVAEYWQNDLGALENYLNKTNFNHSVFDVPL |
| | | HYQFHAASTQGGGYDMRKLLNGTVVSKHPVKAVTFVDNHDTQPGQSLE |
| | | STVQTWFKPLAYAFILTREAGYPQIFYGDMYGTKGASQREIPALKHKIEPI |
| | | LKARIQYAYGAQHDYFDHHDIVGWTREGDSSVANSGLAALITDGPGGTK |
| | | RMYVGRQNAGETWHDITGNRSDSVVINAEGWGEFHVNGGSVSIYVQR |
| 146 | green | TALTEGAKLFEKEIPYITELEGDVEGMKFIIKGEGTGDATTGTIKAKYICTT |
| | fluorescent | GDLPVPWATLVSTLSYGVQCFAKYPSHIKDFFKSAMPEGYTQERTISFEG |
| | protein | DGVYKTRAMVTYERGSIYNRVTLTGENFKKDGHILRKNVAFQCPPSILYI |
| | | LPDTVNNGIRVEFNQAYDIEGVTEKLVTKCSQMNRPLAGSAAVHIPRYHH |
| | | ITYHTKLSKDRDERRDHMCLVEVVKAVDLDTYQ |

What is claimed is:

1. An expression construct comprising a polynucleotide sequence that encodes a protein operably linked to a recombinant secretion signal, wherein said recombinant secretion signal comprises a leader peptide comprising the amino acid sequence of SEQ ID NO: 1 or SEQ ID NO: 2 and a signal peptide comprising the amino acid sequence of SEQ ID NO: 125 or SEQ ID NO: 117.

2. The expression construct of claim 1, wherein said construct comprises multiple copies of the polynucleotide sequence.

3. The expression construct of claim 1, wherein said polynucleotide sequence is operably linked to the pGCW14 promoter of Pichia pastoris or the pGAP promoter of Pichia pastoris.

4. The expression construct of claim 1, wherein said protein is a silk protein.

5. The expression construct of claim 4, wherein said protein comprises SEQ ID NO: 17.

6. The expression construct of claim 4, wherein said protein comprises multiple copies of SEQ ID NO: 17.

7. A recombinant vector comprising the expression construct of claim 1.

8. The recombinant vector of claim 7, wherein said vector comprises multiple copies of said expression construct.

9. The recombinant vector of claim 7, wherein said vector comprises a Pichia autonomously replicating sequence.

10. A recombinant host cell comprising an expression construct comprising a polynucleotide sequence that encodes a protein operably linked to a recombinant secretion signal, wherein said recombinant secretion signal comprises a leader peptide comprising the amino acid sequence of SEQ ID NO: 1 or SEQ ID NO: 2 and a signal peptide comprising the amino acid sequence of SEQ ID NO: 125 or SEQ ID NO: 117.

11. The recombinant host cell of claim 10, wherein said recombinant host cell is a yeast cell.

12. The recombinant host cell of claim 11, wherein said recombinant host cell is a methylotrophic yeast cell.

13. The recombinant host cell of claim 12, wherein said recombinant host cell is Pichia pastoris.

14. The recombinant host cell of claim 10, wherein said expression construct is stably integrated within the genome of said recombinant host cell.

15. The recombinant host cell of claim 10, wherein said expression construct is maintained extrachromosomally in said recombinant host cell.

16. A composition comprising the recombinant host cell of claim 10 and a culture medium.

17. A method for producing a protein, comprising the steps of:
a) culturing the recombinant host cell of claim 10 in a culture medium; and
b) extracting said protein from the culture medium.


SEQUENCE LISTING (154 sequences)

SEQ ID NO: 1 - protein, 70 aa, Saccharomyces cerevisiae
APVNTTTEDETAQIPAEAVIGYLDLEGDFDVAVLPFSNSTNNGLLFINTTIASIAAKEEGVSLDKREAEA

SEQ ID NO: 2 - protein, 70 aa, synthetic construct
APVNTTTEDETAQIPAEAVIGYSDLEGDFDVAVLPFSNSTNNGLLFINTTIASIAAKEEGVSLEKREAEA

SEQ ID NO: 3 - protein, 27 aa, Bos taurus
MDSKGSSQKGSRLLLLLVVSNLLLCSA

SEQ ID NO: 4 - protein, 18 aa, Gallus gallus
MRSLLILVLCFLPLAALG

SEQ ID NO: 5 - protein, 20 aa, Saccharomyces cerevisiae
MKAFTSLLCGLGLSTTLAKA

SEQ ID NO: 6 - protein, 46 aa, Pichia pastoris
MDSEPLLPNPNDSRKPANWRRIIKYISLTLAWIGIFSYVYIYHGTA

SEQ ID NO: 7 - protein, 22 aa, Saccharomyces cerevisiae
MFSLKALLPLALLLVSANQVAA

SEQ ID NO: 8 - protein, 19 aa, Saccharomyces cerevisiae
MLLQAFLFLLAGFAAKISA

SEQ ID NO: 9 - protein, 20 aa, Pichia pastoris
MKLSTNLILAIAAASAVVSA

SEQ ID NO: 10 - protein, 97 aa, synthetic construct
MDSKGSSQKGSRLLLLLVVSNLLLCSAAPVNTTTEDETAQIPAEAVIGYSDLEGDFDVAVLPFSNSTNNGLLFINTTIASIAAKEEGVSLEKREAEA

SEQ ID NO: 11 - protein, 88 aa, synthetic construct
MRSLLILVLCFLPLAALGAPVNTTTEDETAQIPAEAVIGYSDLEGDFDVAVLPFSNSTNNGLLFINTTIASIAAKEEGVSLEKREAEA

SEQ ID NO: 12 - protein, 90 aa, synthetic construct
MKAFTSLLCGLGLSTTLAKAAPVNTTTEDETAQIPAEAVIGYSDLEGDFDVAVLPFSNSTNNGLLFINTTIASIAAKEEGVSLEKREAEA

SEQ ID NO: 13 - protein, 116 aa, synthetic construct
MDSEPLLPNPNDSRKPANWRRIIKYISLTLAWIGIFSYVYIYHGTAAPVNTTTEDETAQIPAEAVIGYSDLEGDFDVAVLPFSNSTNNGLLFINTTIASIAAKEEGVSLEKREAEA

SEQ ID NO: 14 - protein, 92 aa, synthetic construct
MFSLKALLPLALLLVSANQVAAAPVNTTTEDETAQIPAEAVIGYSDLEGDFDVAVLPFSNSTNNGLLFINTTIASIAAKEEGVSLEKREAEA

SEQ ID NO: 15 - protein, 89 aa, synthetic construct
MLLQAFLFLLAGFAAKISAAPVNTTTEDETAQIPAEAVIGYSDLEGDFDVAVLPFSNSTNNGLLFINTTIASIAAKEEGVSLEKREAEA

SEQ ID NO: 16 - protein, 114 aa, synthetic construct
MAKADGSLLYYNPHNPPRRYYFYMAIFAVSVICVLYGPSQQLSSAPVNTTTEDETAQIPAEAVIGYSDLEGDFDVAVLPFSNSTNNGLLFINTTIASIAAKEEGVSLEKREAEA

SEQ ID NO: 17 - protein, 315 aa, synthetic construct
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 18 - protein, 280 aa, synthetic construct
GGQGGRGGFGGLGSQGAGGAGQGGAGAAAAAAAAGGDGGSGLGGYGAGRGHGVGLGGAGGAGAASAAAAAGGQGGRGGFGGLGSQGAGGAGQGGAGAAAAAAAAGGDGGSGLGGYGAGRGHGAGLGGAGGAGAASAAAAAGGQGGRGGFGGLGSQGSGGAGQGGSGAAAAAAAAGGDGGSGLGGYGAGRGYGAGLGGAGGAGAASAAAAAGGQGGRGGFGGLGSQGAGGAGQGGSGAAAAAAAAVADGGSGLGGYGAGRGYGAGLGGAGGAGAASAAAAT

SEQ ID NO: 19 - protein, 278 aa, synthetic construct
GSAPQGAGGPAPQGPSQQGPVSQGPYGPGAAAAAAAAGGYGPGAGQQGPGSQGPGSGGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAA

SEQ ID NO: 20 - protein, 261 aa, synthetic construct
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGGGYGPGAGQQGPGSQGPGSGGQQGPGGQGPYGPSAAAAAAAA

SEQ ID NO: 21 - protein, 258 aa, synthetic construct
GPGARRQGPGSQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 22 - protein, 257 aa, synthetic construct
GPGARRQGPGSQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 23 - protein, 255 aa, synthetic construct
GGYGPGAGQQGPGSGGQQGPGGQGPYGSGQQGPGGAGQQGPGGQGPYGPGAAAAAAAAAGGYGPGAGQQGPGGAGQQGPEGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAA

SEQ ID NO: 24 - protein, 252 aa, synthetic construct
GVFSAGQGATPWENSQLAESFISRFLRFIGQSGAFSPNQLDDMSSIGDTLKTAIEKMAQSRKSSKSKLQALNMAFASSMAEIAVAEQGGLSLEAKTNAIASALSAAFLETTGYVNQQFVNEIKTLIFMIAQASSNEISGSAAAAGGSSGGGGGSGQGGYGQGAYASASAAAAYGSAPQGTGGPASQGPSQQGPVSQPSYGPSATVAVTAVGGRPQGPSAPRQQGPSQQGPGQQGPGGRGPYGPSAAAAAAAA

SEQ ID NO: 25 - protein, 252 aa, synthetic construct
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAAGAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 26 - protein, 252 aa, synthetic construct
GAGAGAGAGAGAGAGAGSGASTSVSTSSSSGSGAGAGAGSGAGSGAGAGSGAGAGAGAGGAGAAFGSGLGLGYGVGLSSAQAQAQAQAAAQAQADAQAQAYAAAQAQAQAQAQAQAAAAAAAAAAAGAGAGAGAGSGAGAGAGSGASTSVSTSSSSGSGAGAGAGSGAGSGAGAGSGAGAGAGAGGAGAGFGSGLGLGYGVGLSSAQAQAQAQAAAQAQADAQAQAYAAAQAQAQAQAQAQAAAAAAAAAAA

SEQ ID NO: 27 - protein, 252 aa, synthetic construct
GAGAGAGAGSGAGAGAGSGASTSVSTSSSSGSGAGAGAGSGAGSGAGAGSGAGAGAGAGGAGAGFGSGLGLGYGVGLSSAQAQAQSAAAARAQADAQAQAYAAAQAQAQAQAQAQAAAAAAAAAAAGAGAGAGAGAGAGAGAGSGASTSVSTSSSSASGAGAGAGSGAGSGAGAGSGAGAGAGAGGAGAGFGSGLGLGYGVGLSSAQAQAQAQAAAQAQAQAQAQALAAAQAQAQAQAQAQAAAATAAAAAA

SEQ ID NO: 28 - protein, 252 aa, synthetic construct
GGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 29 - protein, 251 aa, synthetic construct
GGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 30 - protein, 248 aa, synthetic construct
GHQGPHRKTPWETPEMAENFMNNVRENLEASRIFPDELMKDMEAITNTMIAAVDGLEAQHRSSYASLQAMNTAFASSMAQLFATEQDYVDTEVIAGAIGKAYQQITGYENPHLASEVTRLIQLFREEDDLENEVEISFADTDNAIARAAAGAAAGSAAASSSADASATAEGASGDSGFLFSTGTFGRGGAGAGAGAAAASAAAASAAAAGAEGDRGLFFSTGDFGRGGAGAGAGAAAASAAAASAAAA

SEQ ID NO: 31 - protein, 245 aa, synthetic construct
GGAQKHPSGEYSVATASAAATSVTSGGAPVGKPGVPAPIFYPQGPLQQGPAPGPSNVQPGTSQQGPIGGVGESNTFSSSFASALGGNRGFSGVISSASATAVASAFQKGLAPYGTAFALSAASAAADAYNSIGSGASASAYAQAFARVLYPLLQQYGLSSSADASAFASAIASSFSTGVAGQGPSVPYVGQQQPSIMVSAASASAAASAAAVGGGPVVQGPYDGGQPQQPNIAASAAAAATATSS

SEQ ID NO: 32 - protein, 244 aa, synthetic construct
GGQGGRGGFGGLGSQGEGGAGQGGAGAAAAAAAAGADGGFGLGGYGAGRGYGAGLGGAGGAGAASAAAAAGGQGGRSGFGGLGSQGAGGAGQGGAGAAAAAAAAGADGGSGLGGYGAGRGYGASLGGADGAGAASAAAAAGGQGGRGGFGGLGSQGAGGAGQGGAGAAAAAAAASGDGGSGLGGYGAGRGYGAGLGGAGGAGAASAAAAAGGEGGRGGFGGLGSQGAGGAGQGGSLAAAAAAAA

SEQ ID NO: 33 - protein, 244 aa, synthetic construct
GPGGYGGPGQPGPGQGQYGPGPGQQGPRQGGQQGPASAAAAAAAGPGGYGGPGQQGPRQGQQQGPASAAAAAAAAAAGPRGYGGPGQQGPVQGGQQGPASAAAAAAAAGVGGYGGPGQQGPGQGQYGPGTGQQGQGPSGQQGPAGAAAAAAGGAAGPGGYGGPGQQGPGQGQYGPGTGQQGQGPSGQQGPAGAAAAAAAAAGPGGYGGPGQQGPGQGQYGPGAGQQGQGPGSQQGPASAAAAAA

SEQ ID NO: 34 - protein, 243 aa, synthetic construct
GSGAGQGTGAGAGAAAAAAGAAGSGAGQGAGSGAGAAAAAAAASAAGAGQGAGSGSGAGAAAAAAAAAGAGQGAGSGSGAGAAAAAAAAAAAAQQQQQQQAAAAAAAAAAAAAGSGQGASFGVTQQFGAPSGAASSAAAAAAAAAAAAAGSGAGQEAGTGAGAAAAAAAAGAAGSGAGQGAGSGAGAAAAAAAAASAAGAGQGAGSGSGAGAAAAAAAAAAAAQQQQQQQAAAAAAAAAAAAA

SEQ ID NO: 35 - protein, 242 aa, synthetic construct
GGAQKQPSGESSVATASAAATSVTSAGAPVGKPGVPAPIFYPQGPLQQGPAPGPSYVQPATSQQGPIGGAGRSNAFSSSFASALSGNRGFSEVISSASATAVASAFQKGLAPYGTAFALSAASAAADAYNSIGSGANAFAYAQAFARVLYPLVQQYGLSSSAKASAFASAIASSFSSGAAGQGQSIPYGGQQQPPMTISAASASAGASAAAVKGGQVGQGPYGGQQQSTAASASAAATTATA

SEQ ID NO: 36 - protein, 241 aa, synthetic construct
GADGGSGLGGYGAGRGYGAGLGGADGAGAASAAAAAGGQGGRGGFGRLGSQGAGGAGQGGAGAAAAVAAAGGDGGSGLGGYGAGRGYGAGLGGAGGAGAASAAAAAGGQGGRGGFGGLGSQGAGGAGQGGAGAAASGDGGSGLGGYGAGRGYGAGLGGADGAGAASAASAAGGQGGRGGFGGLGSQGAGGAGQGGAGAAAAAATAGGDGGSGLGGYGAGRGYGAGLGGAGGAGAASAAAAA

SEQ ID NO: 37 - protein, 241 aa, synthetic construct
GAGAGQGGRGGYGQGGFGGQGSGAGAGASAAAGAGAGQGGRGGYGQGGFGGQGSGAGAGASAAAGAGAGQGGRGGYGQGGFGGQGSGAGAGASAAAAAGAGQGGRGGYGQGGLGGSGSGAGAGAGAAAAAAAGAGGYGQGGLGGYGQGAGAGQGGLGGYGSGAGAGASAAAAAGAGGAGQGGLGGYGQGAGAGQGGLGGYGSGAGAGAAAAAAAGAGGSGQGGLGGYGSGGGAGGASAAAA

SEQ ID NO: 38 - protein, 239 aa, synthetic construct
GAYAYAYAIANAFASILANTGLLSVSSAASVASSVASAIATSVSSSSAAAAASASAAAAASAGASAASSASASSSASAAAGAGAGAGAGASGASGAAGGSGGFGLSSGFGAGIGGLGGYPSGALGGLGIPSGLLSSGLLSPAANQRIASLIPLILSAISPNGVNFGVIGSNIASLASQISQSGGGIAASQAFTQALLELVAAFIQVLSSAQIGAVSSSSASAGATANAFAQSLSSAFAG

SEQ ID NO: 39 - protein, 239 aa, synthetic construct
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVRQYGLSSSGKASAFASAIASSFSSGTSGQGPSIGQQQPPVTISAASASAGASAAAVGGGQVGQGPYGGQQQSTAASASAAAATATS

SEQ ID NO: 40 - protein, 239 aa, synthetic construct; identical to SEQ ID NO: 39

SEQ ID NO: 41 - protein, 239 aa, synthetic construct
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 42 - protein, 239 aa, synthetic construct
GGAQKQPSGESSVATASAAATSVTSAGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSNGQQQPPVTISAASASAGASAAAVGGGQVSQGPYGGQQQSTAASASAAAATATS

SEQ ID NO: 43 - protein, 239 aa, synthetic construct
GGAQKQPSGESSVATASAAATSVTSAGAPGGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 44 - protein, 236 aa, synthetic construct
GPGGYGGPGQQGPGQGQQQGPASAAAAAAAAGPGGYGGPGQQGPGQGQQQGPASAAAAAAAAAGPGGYGGPGQQRPGQAQYGRGTGQQGQGPGAQQGPASAAAAAAAGAGLYGGPGQQGPGQGQQQGPASAAAAAAAAAAAGPGGYGGPGQQGPGQAQQQGPASAAAAAAAGPGGYSGPGQQGPGQAQQQGPASAAAAAAAAAGPGGYGGPGQQGPGQGQQQGPASAAAAAAATAA

SEQ ID NO: 45 - protein, 234 aa, synthetic construct
GAGGDGGLFLSSGDFGRGGAGAGAGAAAASAAAASSAAAGARGGSGFGVGTGGFGRGGAGDGASAAAASAAAASAAAAGAGGDSGLFLSSGDFGRGGAGAGAGAAAASAAAASAAAAGTGGVGGLFLSSGDFGRGGAGAGAGAAAASAAAASSAAAGARGGSGFGVGTGGFGRGGPGAGTGAAAASAAAASAAAAGAGGDSGLFLSSEDFGRGGAGAGTGAAAASAAAASAAAA

SEQ ID NO: 46 - protein, 233 aa, synthetic construct
GAGRGYGGGYGGGAAAGAGAGAGAGRGYGGGYGGGAGSGAGSGAGAGGGSGYGRGAGAGAGAGAAAAAGAGAGGAGGYGGGAGAGAGASAAAGAGAGAGGAGGYGGGYGGGAGAGAGAGAAAAAGAGAGAGAGRGYGGGFGGGAGSGAGAGAGAGGGSGYGRGAGGYGGGYGGGAGTGAGAAAATGAGAGAGAGRGYGGGYGGGAGAGAGAGAGAGGGSGYGRGAGAGASVAA

SEQ ID NO: 47 - protein, 231 aa, synthetic construct
GALGQGASVWSSPQMAENFMNGFSMALSQAGAFSGQEMKDFDDVRDIMNSAMDKMIRSGKSGRGAMRAMNAAFGSAIAEIVAANGGKEYQIGAVLDAVTNTLLQLTGNADNGFLNEISRLITLFSSVEANDVSASAGADASGSSGPVGGYSSGAGAAVGQGTAQAVGYGGGAQGVASSAAAGATNYAQGVSTGSTQNVATSTVTTTTNVAGSTATGYNTGYGIGAAAGAAA

SEQ ID NO: 48 - protein, 231 aa, synthetic construct
GGQGGQGGYDGLGSQGAGQGGYGQGGAAAAAAAASGAGSAQRGGLGAGGAGQGYGAGSGGQGGAGQGGAAAATAAAAGGQGGQGGYGGLGSQGSGQGGYGQGGAAAAAAAASGDGGAGQEGLGAGGAGQGYGAGLGGQGGAGQGGAAAAAAAAAGGQGGQGGYGGLGSQGAGQGGYGQGGAAAAAAAASGAGGAGQGGLGAAGAGQGYGAGSGGQGGAGQGGAAAAAAAAA

SEQ ID NO: 49 - protein, 231 aa, synthetic construct
GGQGGQGGYGGLGSQGAGQGGYGQGGVAAAAAAASGAGGAGRGGLGAGGAGQEYGAVSGGQGGAGQGGEAAAAAAAAGGQGGQGGYGGLGSQGAGQGGYGQGGAAAAAAAASGAGGARRGGLGAGGAGQGYGAGLGGQGGAGQGSASAAAAAAAGGQGGQGGYGGLGSQGSGQGGYGQGGAAAAAAAASGAGGAGRGSLGAGGAGQGYGAGLGGQGGAGQGGAAAAASAAA

SEQ ID NO: 50 - protein, 229 aa, synthetic construct
GPGGYGGPGQQGPGQGQYGPGTGQQGQGPGGQQGPVGAAAAAAAAVSSGGYGSQGAGQGGQQGSGQRGPAAAGPGGYSGPGQQGPGQGGQQGPASAAAAAAAAAGPGGYGGSGQQGPGQGRGTGQQGQGPGGQQGPASAAAAAAAGPGGYGGPGQQGPGQGQYGPGTGQQGQGPASAAAAAAAGPGGYGGPGQQGPGQGQYGPGTGQQGQGPGGQQGPGGASAAAAAAA

SEQ ID NO: 51 - protein, 228 aa, synthetic construct
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGPGAGRQGPGSQGPGSGGQQGPGGQGPYGPSAAAAAAAA

SEQ ID NO: 52 - protein, 225 aa, synthetic construct
GQGGQGGQGGLGQGGYGQGAGSSAAAAAAAAAAAAAAGRGQGGYGQGSGGNAAAAAAAAAAAASGQGSQGGQGGQGQGGYGQGAGSSAAAAAAAAAAAAASGRGQGGYGQGAGGNAAAAAAAAAAAAAAGQGGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAA

SEQ ID NO: 53 - protein, 225 aa, synthetic construct
GRGPGGYGPGQQGPGGPGAAAAAAGPGGYGPGGYGPGQQGPGGPGAAAAAAAGRGPGGYGPGQQGPGQQGPGGSGAAAAAAGRGPGGYGPGQQGPGGPGAAAAAAGPGGYGPGQQGPGAAAAAAAAGRGPGGYGPGQQGPGGPGAAAAAAAGRGPGGYGPGQQGPGQQGPGGSGAAAAAAGRGPGGYGPGQQGPGGPGAAAAAAGPGGYGPGQQGPGAAAAAAAA

SEQ ID NO: 54 - protein, 225 aa, synthetic construct
GRGPGGYGPGQQGPGGSGAAAAAAGRGPGGYGPGQQGPGGPGAAAAAAGPGGYGPGQQGTGAAAAAAAGSGAGGYGPGQQGPGGPGAAAAAAGPGGYGPGQQGPGAAAAAAAGSGPGGYGPGQQGPGGSSAAAAAAGPGRYGPGQQGPGAAAAASAGRGPGGYGPGQQGPGGPGAAAAAAGPGGYGPGQQGPGAAAAAAAGSGPGGYGPGQQGPGGPGAAAAAAA

SEQ ID NO: 55 - protein, 219 aa, synthetic construct
GAAATAGAGASVAGGYGGGAGAAAGAGAGGYGGGYGAVAGSGAGAAAAASSGAGGAAGYGRGYGAGSGAGAGAGTVAAYGGAGGVATSSSSATASGSRIVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 56 - protein, 219 aa, synthetic construct
GGQGGFSGQGQGGFGPGAGSSAAAAAAAAAAARQGGQGQGGFGQGAGGNAAAAAAAAAAAAAAQQGGQGGFSGRGQGGFGPGAGSSAAAAAAGQGGQGQGGFGQGAGGNAAAAAAAAAAAAAAAGQGGQGRGGFGQGAGGNAAAAAAAAAAAAAAAQQGGQGGFGGRGQGGFGPGAGSSAAAAAAGQGGQGRGGFGQGAGGNAAAASAAAAASAAAAGQ

SEQ ID NO: 57 - protein, 218 aa, synthetic construct
GGYGPGAGQQGPGGAGQQGPGSQGPGGAGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGLGPYGPSAAAAAAAAGGYGPGAGQQGPGSQGPGSGGQQRPGGLGPYGPSAAAAAAAAGGYGPGAGQQGPGSQGPGSGGQQRPGGLGPYGPSAAAAAAAA

SEQ ID NO: 58 - protein, 217 aa, synthetic construct
GAGAGGGYGGGYSAGGGAGAGSGAAAGAGAGRGGAGGYSAGAGTGAGAAAGAGTAGGYSGGYGAGASSSAGSSFISSSSMSSSQATGYSSSSGYGGGAASAAAGAGAAAGGYGGGYGAGAGAGAAAASGATGRVANSLGAMASGGINALPGVFSNIFSQVSAASGGASGGAVLVQALTEVIALLLHILSSASIGNVSSQGLEGSMAIAQQAIGAYAG

SEQ ID NO: 59 - protein, 216 aa, synthetic construct
GAGAGGAGGYAQGYGAGAGAGAGAGTGAGGAGGYGQGYGAGSGAGAGGAGGYGAGAGAGAGAGDASGYGQGYGDGAGAGAGAAAAAGAAAGARGAGGYGGGAGAGAGAGAGAAGGYGQGYGAGAGEGAGAGAGAGAVAGAGAAAAAGAGAGAGGAEGYGAGAGAGGAGGYGQSYGDGAAAAAGSGAGAGGSGGYGAGAGAGSGAGAAGGYGGGAGA

SEQ ID NO: 60 - protein, 216 aa, synthetic construct
GPGGYGPGQQGPGGYGPGQQGPGRYGPGQQGPSGPGSAAAAAAGSGQQGPGGYGPRQQGPGGYGQGQQGPSGPGSAAAASAAASAESGQQGPGGYGPGQQGPGGYGPGQQGPGGYGPGQQGPSGPGSAAAAAAAASGPGQQGPGGYGPGQQGPGGYGPGQQGPSGPGSAAAAAAAASGPGQQGPGGYGPGQQGPGGYGPGQQGLSGPGSAAAAAAA

SEQ ID NO: 61 - protein, 216 aa, synthetic construct
GRGPGGYGQGQQGPGGPGAAAAAAGPGGYGPGQQGPGAAAAAAAGSGPGGYGPGQQGPGRSGAAAAAAAAGRGPGGYGPGQQGPGGPGAAAAAAGPGGYGPGQQGPGAAAAASAGRGPGGYGPGQQGPGGSGAAAAAAGRGPGGYGPGQQGPGGPGAAAAAAAGRGPGGYGPGQQGPGQQGPGGSGAAAAAAGRGPGGYGPGQQGPGGPGAAAAAA

SEQ ID NO: 62 - protein, 214 aa, synthetic construct
GVGAGGEGGYDQGYGAGAGAGSGGGAGGAGGYGGGAGAGSGGGAGGAGGYGGGAGAGAGAGAGGAGGYGGGAGAGTGARAGAGGVGGYGQSYGAGASAAAGAGVGAGGAGAGGAGGYGQGYGAGAGIGAGDAGGYGGGAGAGASAGAGGYGGGAGAGAGGVGGYGKGYGAGSGAGAAAAAGAGAGSAGGYGRGDGAGAGGASGYGQGYGAGAAA

SEQ ID NO: 63 - protein, 212 aa, synthetic construct
GYGAGAGRGYGAGAGAGAGAVAASGAGAGAGYGAGAGAGAGAGYGAGAGRGYGAGAGAGAGSGAASGAGAGAGYGAGAGAGAGYGAGAGSGYGTGAGAGAGAAAAGGAGAGAGYGAGAGRGYGAGAGAGAASGAGAGAGAGAASGAGAGSGYGAGAAAAGGAGAGAGGGYGAGAGRGYGAGAGAGAGAGSGSGSAAGYGQGYGSGSGAGAAA

SEQ ID NO: 64 - protein, 198 aa, synthetic construct
GQGTDSSASSVSTSTSVSSSATGPDTGYPVGYYGAGQAEAAASAAAAAAASAAEAATIAGLGYGRQGQGTDSSASSVSTSTSVSSSATGPDMGYPVGNYGAGQAEAAASAAAAAAASAAEAATIASLGYGRQGQGTDSSASSVSTSTSVSSSATGPGSRYPVRDYGADQAEAAASAAAAAAAAASAAEEIASLGYGRQ

SEQ ID NO: 65 - protein, 198 aa, synthetic construct
GQGTDSVASSASSSASASSSATGPDTGYPVGYYGAGQAEAAASAAAAAAASAAEAATIAGLGYGRQGQGTDSSASSVSTSTSVSSSATGPGSRYPVRDYGADQAEAAASATAAAAAAASAAEEIASLGYGRQGQGTDSVASSASSSASASSSATGPDTGYPVGYYGAGQAEAAASAAAAAAASAAEAATIAGLGYGRQ

SEQ ID NO: 66 - protein, 195 aa, synthetic construct
GQGGQGGYGGLGQGGYGQGAGSSAAAAAAAAAAAAAGGQGGQGQGRYGQGAGSSAAAAAAAAAAAAAAGRGQGGYGQGSGGNAAAAAAAAAAAASGQGSQGGQGGQGQGGYGQGAGSSAAAAAAAAAAAAASGRGQGGYGQGAGGNAAAAAAAAAAAAAAGQGGQGGYGGLGQGGYGQGAGSSAAAAAAAAAAAA

SEQ ID NO: 67 - protein, 193 aa, synthetic construct
GGLGGQGGLGGLGSQGAGLGGYGQGGAGQGGAAAAAAAAGGLGGQGGRGGLGSQGAGQGGYGQGGAGQGGAAAAAAAAGGLGGQGGLGALGSQGAGQGGAGQGGYGQGGAAAAAAGGLGGQGGLGGLGSQGAGQGGYGQGGAGQGGAAAAAAAAGGLGGQGGLGGLGSQGAGPGGYGQGGAGQGGAAAAAAAA

SEQ ID NO: 68 - protein, 192 aa, synthetic construct
GGQGRGGFGQGAGGNAAAAAAAAAAAAAAQQVGQFGFGGRGQGGFGPFAGSSAAAAAAASAAAGQGGQGQGGFGQGAGGNAAAAAAAAAAAARQGGQGQGGFSQGAGGNAAAAAAAAAAAAAAAQQGGQGGFGGRGQGGFGPGAGSSAAAAAAATAAAGQGGQGRGGFGQGAGSNAAAAAAAAAAAAAAAGQ

SEQ ID NO: 69 - protein, 190 aa, synthetic construct
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 70 - protein, 188 aa, synthetic construct
GGAGQRGYGGLGNQGAGRGGLGGQGAGAAAAAAAGGAGQGGYGGLGNQGAGRGGQGAAAAAGGAGQGGYGGLGSQGAGRGGQGAGAAAAAAVGAGQEGIRGQGAGQGGYGGLGSQGSGRGGLGGQGAGAAAAAAGGAGQGGLGGQGAGQGAGAAAAAAGGVRQGGYGGLGSQGAGRGGQGAGAAAAAA

SEQ ID NO: 71 - protein, 186 aa, synthetic construct
GGAGQGGLGGQGAGQGAGASAAAAGGAGQGGYGGLGSQGAGRGGEGAGAAAAAAGGAGQGGYGGLGGQGAGQGGYGGLGSQGAGRGGLGGQGAGAAAAGGAGQGGLGGQGAGQGAGAAAAAAGGAGQGGYGGLGSQGAGRGGLGGQGAGAVAAAAAGGAGQGGYGGLGSQGAGRGGQGAGAAAAAA

SEQ ID NO: 72 - protein, 182 aa, synthetic construct
GAGAGAGAGSGAGAAGGYGGGAGAGVGAGGAGGYDQGYGAGAGAGSGAGAGGAGGYGGGAGAGADAGAGGAGGYGGGAGAGAGARAGAGGVGGYGQSYGAGAGAGAGVGAGGAGAGGADGYGQGYGAGAGTGAGDAGGYGGGAGAGASAGAGGYGGGAGAGGVGVYGKGYGSGSGAGAAAAA

SEQ ID NO: 73 - protein, 182 aa, synthetic construct
GGAGGYGVGQGYGAGAGAGAAAGAGAGGAGGYGAGQGYGAGAGVGAAAAAGAGAGVGGAGGYGRGAGAGAGAGAGAAAGAGAGAAAGAGAGGAGGYGAGQGYGAGAGVGAAAAAGAGAGVGGAGGYGRGAGAGAGAGAGGAGGYGRGAGAGAGAGAGAGGAGGYGAGQGYGAGAGAGAAAAA

SEQ ID NO: 74 - protein, 182 aa, synthetic construct
GEAFSASSASSAVVFESAGPGEEAGSSGDGASAAASAAAAAGAGSGRRGPGGARSRGGAGAGAGAGSGVGGYGSGSGAGAGAGAGAGAGGEGGFGEGQGYGAGAGAGFGSGAGAGAGAGSGAGAGEGVGSGAGAGAGAGFGVGAGAGAGAGAGFGSGAGAGSGAGAGYGAGRAGGRGRGGRG

SEQ ID NO: 75 - protein, 182 aa, synthetic construct
GEAFSASSASSAVVFESAGPGEEAGSSGGGASAAASAAAAAGAGSGRRGPGGARSRGGAGAGAGAGSGVGGYGSGSGAGAGAGAGAGAGGEGGFGEGQGYGAGAGAGFGSGAGAGAGAGSGAGAGEGVGSGAGAGAGAGFGVGAGAGAGAGAGFGSGAGAGSGAGAGYGAGRAGGRGRGGRG

SEQ ID NO: 76 - protein, 182 aa, synthetic construct
GNGLGQALLANGVLNSGNYLQLANSLAYSFGSSLSQYSSSAAGASAAGAASGAAGAGAGAASSGGSSGSASSSTTTTTTTSTSAAAAAAAAAAAASAAASTSASASASASASASAFSQTFVQTVLQSAAFGSYFGGNLSLQSAQAAASAAAQAAAQQIGLGSYGYALANAVASAFASAGANA

SEQ ID NO: 77 - protein, 182 aa, synthetic construct; identical to SEQ ID NO: 76

SEQ ID NO: 78 - protein, 182 aa, synthetic construct; identical to SEQ ID NO: 76

SEQ ID NO: 79 - protein, 180 aa, synthetic construct
GASGAGQGQGYGQQGQGGSSAAAAAAAAAAAAAAAQGQGQGYGQQGQGSAAAAAAAAAAGASGAGQGQGYGQQGQGSAAAAAAAAAAGASGAGQGQGYGQQGQGGSSAAAAAAAAAAAAAAAAQGQGYGQQGQGSAAAAAAAAAGASGAGQGQGYGQQGQGGSSAAAAAAAAAAAAAAAA

SEQ ID NO: 80 - protein, 179 aa, synthetic construct
GRGQGGYGQGSGGNAAAAAAAGQGGFGGQEGNGQGAGSAAAAAAAAAAAAGGSGQGRYGGRGQGGYGQGAGAAASAAAAAAAAAAGQGGFGGQEGNGQGAGSAAAAAAAAAAAAGGSGQGGYGGRGQGGYGQGAGAAAAAAAAAAAAAAGQGGQGGFGSQGGNGQGAGSAAAAAAAAAA

SEQ ID NO: 81 - protein, 178 aa, synthetic construct
GQNTPWSSTELADAFINAFMNEAGRTGAFTADQLDDMSTIGDTIKTAMDKMARSNKSSKGKLQALNMAFASSMAEIAAVEQGGLSVDAKTNAIADSLNSAFYQTTGAANPQFVNEIRSLINMFAQSSANEVSYGGGYGGQSAGAAASAAAAGGGGQGGYGNLGGQGAGAAAAAAASAA

SEQ ID NO: 82 - protein, 178 aa, synthetic construct
GQNTPWSSTELADAFINAFLNEAGRTGAFTADQLDDMSTIGDTLKTAMDKMARSNKSSQSKLQALNMAFASSMAEIAAVEQGGLSVAEKTNAIADSLNSAFYQTTGAVNVQFVNEIRSLISMFAQASANEVSYGGGYGGGQGGQSAGAAAAAASAGAGQGGYGGLGGQGAGSAAAAAA

SEQ ID NO: 83 - protein, 177 aa, synthetic construct
GGQGGQGGYGGLGSQGAGQGGYGQGGAAAAAASAGGQGGQGGYGGLGSQGAGQGGYGGGAFSGQQGGAASVATASAAASRLSSPGAASRVSSAVTSLVSSGGPTNSAALSNTISNVVSQISSSNPGLSGCDVLVQALLEIVSALVHILGSANIGQVNSSGVGRSASIVGQSINQAFS

SEQ ID NO: 84 - protein, 177 aa, synthetic construct
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 85 - protein, 175 aa, synthetic construct
GASSAAAAAAATATSGGAPGGYGGYGPGIGGAFVPASTTGTGSGSGSGAGAAGSGGLGGLGSSGGSGGLGGGNGGSGASAAASAAAASSSPGSGGYGPGQGVGSGSGSGAAGGSGTGSGAGGPGSGGYGGPQFFASAYGGQGLLGTSGYGNGQGGASGTGSGGVGGSGSGAGSNS

SEQ ID NO: 86 - protein, 174 aa, synthetic construct
GQPIWTNPNAAMTMTNNLVQCASRSGVLTADQMDDMGMMADSVNSQMQKMGPNPPQHRLRAMNTAMAAEVAEVVATSPPQSYSAVLNTIGACLRESMMQATGSVDNAFTNEVMQLVKMLSADSANEVSTASASGASYATSTSSAVSSSQATGYSTAAGYGNAAGAGAGAAAAVS

SEQ ID NO: 87 - protein, 174 aa, synthetic construct
GQKIWTNPDAAMAMTNNLVQCAGRSGALTADQMDDLGMVSDSVNSQVRKMGANAPPHKIKAMSTAVAAGVAEVVASSPPQSYSAVLNTIGGCLRESMMQVTGSVDNTFTTEMMQMVNMFAADNANEVSASASGSGASYATGTSSAVSTSQATGYSTAGGYGTAAGAGAGAAAAA

SEQ ID NO: 88 - protein, 174 aa, synthetic construct
GSGYGAGAGAGAGSGYGAGAGAGSGYGAGAGAGAGSGYVAGAGAGAGAGSGYGAGAGAGAGSSYSAGAGAGAGSGYGAGSSASAGSAVSTQTVSSSATTSSQSAAAATGAAYGTRASTGSGASAGAAASGAGAGYGGQAGYGQGGGAAAYRAGAGSQAAYGQGASGSSGAAAAA

SEQ ID NO: 89 - protein, 171 aa, synthetic construct
GGQGGRGGFGGLSSQGAGGAGQGGSGAAAAAAAAGGDGGSGLGDYGAGRGYGAGLGGAGGAGVASAAASAAASRLSSPSAASRVSSAVTSLISGGGPTNPAALSNTFSNVVYQISVSSPGLSGCDVLIQALLELVSALVHILGSAIIGQVNSSAAGESASLVGQSVYQAFS

SEQ ID NO: 90 - protein, 169 aa, synthetic construct
GVGQAATPWENSQLAEDFINSFLRFIAQSGAFSPNQLDDMSSIGDTLKTAIEKMAQSRKSSKSKLQALNMAFASSMAEIAVAEQGGLSLEAKTNAIANALASAFLETTGFVNQQFVSEIKSLIYMIAQASSNEISGSAAAAGGGSGGGGGSGQGGYGQGASASASAAAA

SEQ ID NO: 91 - protein, 169 aa, synthetic construct
GGGDGYGQGGYGNQRGVGSYGQGAGAGAAATSAAGGAGSGRGGYGEQGGLGGYGQGAGAGAASTAAGGGDGYGQGGYGNQGGRGSYGQGSGAGAGAAVAAAAGGAVSGQGGYDGEGGQGGYGQGSGAGAAVAAASGGTGAGQGGYGSQGSQAGYGQGAGFRAAAATAAA

SEQ ID NO: 92 - protein, 168 aa, synthetic construct
GAGAGYGGQVGYGQGAGASAGAAAAGAGAGYGGQAGYGQGAGGSAGAAAAGAGAGRQAGYGQGAGASARAAAAGAGTGYGQGAGASAGAAAAGAGAGSQVGYGQGAGASSGAAAAAGAGAGYGGQVGYEQGAGASAGAEAAASSAGAGYGGQAGYGQGAGASAGAAAA

SEQ ID NO: 93 - protein, 166 aa, synthetic construct
GGAGQGGYGGLGGQGAGQGGLGGQRAGAAAAAAGGAGQGGYGGLGSQGAGRGGYGGVGSGASAASAAASRLSSPEASSRVSSAVSNLVSSGPTNSAALSSTISNVVSQISASNPGLSGCDVLVQALLEVVSALIQILGSSSIGQVNYGTAGQAAQIVGQSVYQALG

SEQ ID NO: 94 - protein, 166 aa, synthetic construct
GGYGPGSGQQGPGGAGQQGPGGQGPYGPGSSSAAAVGGYGPSSGLQGPAGQGPYGPGAAASAAAAAGASRLSSPQASSRVSSAVSSLVSSGPTNSAALTNTISSVVSQISASNPGLSGCDVLIQALLEIVSALVHILGYSSIGQINYDAAAQYASLVGQSVAQALA

SEQ ID NO: 95 - protein, 166 aa, synthetic construct
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 96 - protein, 165 aa, synthetic construct
GQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 97 - protein, 164 aa, synthetic construct
GGAGQGGYGGLGGQGSGAAAAGTGQGGYGSLGGQGAGAAGAAAAAVGGAGQGGYGGVGSAAASAAASRLSSPEASSRVSSAVSNLVSSGPTNSAALSNTISNVVSQISSSNPGLSGCDVLVQALLEVVSALIHILGSSSIGQVNYGSAGQATQIVGQSVYQALG

SEQ ID NO: 98 - protein, 164 aa, synthetic construct
GAGAGGAGGYGAGQGYGAGAGAGAAAGAGAGGARGYGARQGYGSGAGAGAGARAGGAGGYGRGAGAGAAAASGAGAGGYGAGQGYGAGAGAVASAAAGAGSGAGGAGGYGRGAGAVAGAGAGGAGGYGAGAGAAAGVGAGGSGGYGGRQGGYSAGAGAGAAAAA

SEQ ID NO: 99 - protein, 163 aa, synthetic construct
GQGGQGGYGGLGQGGYGQGAGSSAAAAAAAAAAAGRGQGGYGQGSGGNAAAAAAAAAAAASGQGGQGGQGGQGQGGYGQGAGSSAAAAAAAAAAAAAAAGRGQGGYGQGAGGNAAAAAAAAAAAASGQGGQGGQGGQGQGGYGQGAGSSAAAAAAAAAAAAAA

SEQ ID NO: 100 - protein, 162 aa, synthetic construct
GGYGPGSGQQGPGQQGPGQQGPGQQGPYGAGASAAAAAAGGYGPGSGQQGPGVRVAAPVASAAASRLSSSAASSRVSSAVSSLVSSGPTTPAALSNTISSAVSQISASNPGLSGCDVLVQALLEVVSALVHILGSSSVGQINYGASAQYAQMVGQSVTQALV

SEQ ID NO: 101 - protein, 161 aa, synthetic construct
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 102 - protein, 160 aa, synthetic construct
GAGAGRGGYGRGAGAGGYGGQGGYGAGAGAGAAAAAGAGAGGYGDKEIACWSRCRYTVASTTSRLSSAEASSRISSAASTLVSGGYLNTAALPSVISDLFAQVGASSPGVSDSEVLIQVLLEIVSSLIHILSSSSVGQVDFSSVGSSAAAVGQSMQVVMG

SEQ ID NO: 103 - protein, 160 aa, synthetic construct
GAGAGAGGAGGYGRGAGAGAGAGAGAAAGQGYGSGAGAGAGASAGGAGSYGRGAGAGAAAASGAGAGGYGAGQGYGAGAGAVASAAAGAGSGAGGAGGYGRGAVAGSGAGAGAGAGGAGGYGAGAGAGAAAGAVAGGSGGYGGRQGGYSAGAGAGAAAAA

SEQ ID NO: 104 - protein, 159 aa, synthetic construct
GPGGYGPVQQGPSGPGSAAGPGGYGPAQQGPARYGPGSAAAAAAAAGSAGYGPGPQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAFS

SEQ ID NO: 105 - protein, 156 aa, synthetic construct
GTGGVGGLFLSSGDFGRGGAGAGAGAAAASAAAASSAAAGARGGSGFGVGTGGFGRGGAGAGTGAAAASAAAASAAAAGAGGDGGLFLSSGDFGRGGAGAGAGAAAASAAAASSAAAGARGGSGFGVGTGGFGRGGAGDGASAAAASAAAASAAAA

SEQ ID NO: 106 - protein, 153 aa, synthetic construct
GGYGPGAGQQGPGGAGQQGPGGQGPYGPSVAAAASAAGGYGPGAGQQGPVASAAVSRLSSPQASSRVSSAVSSLVSSGPTNPAALSNAMSSVVSQVSASNPGLSGCDVLVQALLEIVSALVHILGSSSIGQINYAASSQYAQMVGQSVAQALA

SEQ ID NO: 107 - protein, 153 aa, synthetic construct
GGAGQGGYGGLGSQGAGRGGYGGQGAGAAAAATGGAGQGGYGGVGSGASAASAAASRLSSPQASSRVSSAVSNLVASGPTNSAALSSTISNAVSQIGASNPGLSGCDVLIQALLEVVSALIHILGSSSIGQVNYGSAGQATQIVGQSVYQALG

SEQ ID NO: 108 - protein, 153 aa, synthetic construct
GGAGQGGYGGLGSQGAGRGGYGGQGAGAAVAAIGGVGQGGYGGVGSGASAASAAASRLSSPEASSRVSSAVSNLVSSGPTNSAALSSTISNVVSQIGASNPGLSGCDVLIQALLEVVSALVHILGSSSIGQVNYGSAGQATQIVGQSVYQALG

SEQ ID NO: 109 - protein, 152 aa, synthetic construct
GASGGYGGGAGEGAGAAAAAGAGAGGAGGYGGGAGSGAGAVARAGAGGAGGYGSGIGGGYGSGAGAAAGAGAGGAGAYGGGYGTGAGAGARGADSAGAAAGYGGGVGTGTGSSAGYGRGAGAGAGAGAAAGSGAGAAGGYGGGYGAGAGAGA

SEQ ID NO: 110 - protein, 152 aa, synthetic construct
GAGSGQGGYGGQGGLGGYGQGAGAGAAAGASGSGSGGAGQGGLGGYGQGAGAGAAAAAAGASGAGQGGFGPYGSSYQSSTSYSVTSQGAAGGLGGYGQGSGAGAAAAGAAGQGGQGGYGQGAGAGAGAGAGQGGLGGYGQGAGSSAASAAAA

SEQ ID NO: 111 - protein, 151 aa, synthetic construct
GGAGQGGYGGLGGQGVGRGGLGGQGAGAAAAGGAGQGGYGGVGSGASAASAAASRLSSPQASSRLSSAVSNLVATGPTNSAALSSTISNVVSQIGASNPGLSGCDVLIQALLEVVSALIQILGSSSIGQVNYGSAGQATQIVGQSVYQALG

SEQ ID NO: 112 - protein, 150 aa, synthetic construct
GAGSGGAGGYGRGAGAGAGAAAGAGAGAGSYGGQGGYGAGAGAGAAAAAGAGAGAGGYGRGAGAGAGAGAGAAARAGAGAGGAGYGGQGGYGAGAGAGAAAAAGAGAGGAGGYGRGAGAGAGAAAGAGAGAGGYGGQSGYGAGAGAAAAA

SEQ ID NO: 113 - protein, 150 aa, synthetic construct
GASGAGQGQGYGQQGQGGSSAAAAAAAAAAAQGQGQGYGQQGQGYGQQGQGGSSAAAAAAAAAAAAAQGQGQGYGQQGQGSAAAAAAAAAGASGAGQGQGYGQQGQGGSSAAAAAAAAAAAAAAAQGQGYGQQGQGSAAAAAAAAAAAAA

SEQ ID NO: 114 - protein, 945 aa, synthetic construct
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 115 - protein, 89 aa, synthetic construct
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 116 - protein, 48 aa, Saccharomyces cerevisiae
MEGGEEEVERIPDELFDTKKKHLLDKLIRVGIILVLLIWGTVLLLKSI

SEQ ID NO: 117 - protein, 43 aa, Saccharomyces cerevisiae
MFFNRLSAGKLLVPLSVVLYALFVVILPLQNSFHSSNVLVRGA

SEQ ID NO: 118 - protein, 26 aa, Saccharomyces cerevisiae
MPFGIDNTDFTVLAGLVLAVLLYVKR

SEQ ID NO: 119 - protein, 17 aa, Saccharomyces cerevisiae
MKPQCILISLLVNLAYA

SEQ ID NO: 120 - protein, 24 aa, Saccharomyces cerevisiae
MISANSLLISTLCAFAIATPLSKR

SEQ ID NO: 121 - protein, 19 aa, Saccharomyces cerevisiae
MLQSVVFFALLTFASSVSA

SEQ ID NO: 122 - protein, 21 aa, Rattus norvegicus
MRLAVVCLCLFGLASCLPVKV

SEQ ID NO: 123 - protein, 31 aa, Pichia pastoris
MLSLKPSWLTLAALMYAMLLVVVPFAKPVRA

SEQ ID NO: 124 - protein, 23 aa, Pichia pastoris
MSFSSNVPQLFLLLVLLTNIVSG

SEQ ID NO: 125 - protein, 16 aa, Pichia pastoris
MNLYLITLLFASLCSA

SEQ ID NO: 126 - protein, 19 aa, Saccharomyces cerevisiae
MHWAAAVAIFFIVVTKFLQ

SEQ ID NO: 127 - protein, 18 aa, Pichia pastoris
MRFSNFLTVSALLTGALG

SEQ ID NO: 128 - protein, 20 aa, Saccharomyces cerevisiae
MSLLYIILLFTQFLLLPTDA

SEQ ID NO: 129 - protein, 44 aa, Pichia pastoris
MAKADGSLLYYNPHNPPRRYYFYMAIFAVSVICVLYGPSQQLSS

SEQ ID NO: 130 - protein, 90 aa, synthetic construct
MKLSTNLILAIAAASAVVSAAPVNTTTEDETAQIPAEAVIGYSDLEGDFDVAVLPFSNSTNNGLLFINTTIASIAAKEEGVSLEKREAEA

SEQ ID NO: 131 - protein, 118 aa, synthetic construct
MEGGEEEVERIPDELFDTKKKHLLDKLIRVGIILVLLIWGTVLLLKSIAPVNTTTEDETAQIPAEAVIGYSDLEGDFDVAVLPFSNSTNNGLLFINTTIASIAAKEEGVSLEKREAEA

SEQ ID NO: 132 - protein, 113 aa, synthetic construct
MFFNRLSAGKLLVPLSVVLYALFVVILPLQNSFHSSNVLVRGAAPVNTTTEDETAQIPAEAVIGYSDLEGDFDVAVLPFSNSTNNGLLFINTTIASIAAKEEGVSLEKREAEA

SEQ ID NO: 133 - protein, 96 aa, synthetic construct
MPFGIDNTDFTVLAGLVLAVLLYVKRAPVNTTTEDETAQIPAEAVIGYSDLEGDFDVAVLPFSNSTNNGLLFINTTIASIAAKEEGVSLEKREAEA

SEQ ID NO: 134 - protein, 87 aa, synthetic construct
MKPQCILISLLVNLAYAAPVNTTTEDETAQIPAEAVIGYSDLEGDFDVAVLPFSNSTNNGLLFINTTIASIAAKEEGVSLEKREAEA

SEQ ID NO: 135 - protein, 94 aa, synthetic construct
MISANSLLISTLCAFAIATPLSKRAPVNTTTEDETAQIPAEAVIGYSDLEGDFDVAVLPFSNSTNNGLLFINTTIASIAAKEEGVSLEKREAEA

SEQ ID NO: 136 - protein, 89 aa, synthetic construct
MLQSVVFFALLTFASSVSAAPVNTTTEDETAQIPAEAVIGYSDLEGDFDVAVLPFSNSTNNGLLFINTTIASIAAKEEGVSLEKREAEA

SEQ ID NO: 137 - protein, 91 aa, synthetic construct
MRLAVVCLCLFGLASCLPVKVAPVNTTTEDETAQIPAEAVIGYSDLEGDFDVAVLPFSNSTNNGLLFINTTIASIAAKEEGVSLEKREAEA

SEQ ID NO: 138 - protein, 101 aa, synthetic construct
MLSLKPSWLTLAALMYAMLLVVVPFAKPVRAAPVNTTTEDETAQIPAEAVIGYSDLEGDFDVAVLPFSNSTNNGLLFINTTIASIAAKEEGVSLEKREAEA

SEQ ID NO: 139 - protein, 93 aa, synthetic construct
MSFSSNVPQLFLLLVLLTNIVSGAPVNTTTEDETAQIPAEAVIGYSDLEGDFDVAVLPFSNSTNNGLLFINTTIASIAAKEEGVSLEKREAEA

SEQ ID NO: 140 - protein, 86 aa, synthetic construct
MNLYLITLLFASLCSAAPVNTTTEDETAQIPAEAVIGYSDLEGDFDVAVLPFSNSTNNGLLFINTTIASIAAKEEGVSLEKREAEA

SEQ ID NO: 141 - protein, 89 aa, synthetic construct
MHWAAAVAIFFIVVTKFLQAPVNTTTEDETAQIPAEAVIGYSDLEGDFDVAVLPFSNSTNNGLLFINTTIASIAAKEEGVSLEKREAEA

SEQ ID NO: 142 - protein, 88 aa, synthetic construct
MRFSNFLTVSALLTGALGAPVNTTTEDETAQIPAEAVIGYSDLEGDFDVAVLPFSNSTNNGLLFINTTIASIAAKEEGVSLEKREAEA

SEQ ID NO: 143 - protein, 90 aa, synthetic construct
MSLLYIILLFTQFLLLPTDAAPVNTTTEDETAQIPAEAVIGYSDLEGDFDVAVLPFSNSTNNGLLFINTTIASIAAKEEGVSLEKREAEA

SEQ ID NO: 144 - protein, 37 aa, synthetic construct
APVAPAEEAANHLHKRAYYTDTTKTHTFTEVVTVYRT

SEQ ID NO: 145 - protein, 483 aa, synthetic construct
ANLNGTLMQYFEWYMPNDGQHWKRLQNDSAYLAEHGITAVWIPPAYKGTSQDDVGYGAYDLYDLGEFHQKGTVRTKYGTKGELQSAINSLHSRDINVYGDVVINHKGGADATEDVTAVEVDPADRNRVTSGEQRIKAWTHFQFPGRGSTYSDFKWHWYHFDGTDWDESRKLNRIYKFQGKAWDWEVSNVNGNYDYLMYADIDYDHPDATAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPVKAVTFVDNHDTQPGQSLESTVQTWFKPLAYAFILTREAGYPQIFYGDMYGTKGASQREIPALKHKIEPILKARIQYAYGAQHDYFDHHDIVGWTREGDSSVANSGLAALITDGPGGTKRMYVGRQNAGETWHDITGNRSDSVVINAEGWGEFHVNGGSVSIYVQR

SEQ ID NO: 146 - protein, 235 aa, synthetic construct
TALTEGAKLFEKEIPYITELEGDVEGMKFIIKGEGTGDATTGTIKAKYICTTGDLPVPWATLVSTLSYGVQCFAKYPSHIKDFFKSAMPEGYTQERTISFEGDGVYKTRAMVTYERGSIYNRVTLTGENFKKDGHILRKNVAFQCPPSILYILPDTVNNGIRVEFNQAYDIEGVTEKLVTKCSQMNRPLAGSAAVHIPRYHHITYHTKLSKDRDERRDHMCLVEVVKAVDLDTYQ

SEQ ID NO: 147 - protein, 64 aa, synthetic construct
  VARIANT 4..8: note = This region may encompass "SGGQQ," "GAGQQ," "GQGPY," "AGQQ" or "SQ," wherein some positions may be absent
  VARIANT 12..16: note = This region may encompass "SGGQQ," "GAGQQ," "GQGPY," "AGQQ" or "SQ," wherein some positions may be absent
  VARIANT 20..24: note = This region may encompass "SGGQQ," "GAGQQ," "GQGPY," "AGQQ" or "SQ," wherein some positions may be absent
  VARIANT 28..32: note = This region may encompass "SGGQQ," "GAGQQ," "GQGPY," "AGQQ" or "SQ," wherein some positions may be absent
  VARIANT 36..40: note = This region may encompass "SGGQQ," "GAGQQ," "GQGPY," "AGQQ" or "SQ," wherein some positions may be absent
  VARIANT 44..48: note = This region may encompass "SGGQQ," "GAGQQ," "GQGPY," "AGQQ" or "SQ," wherein some positions may be absent
  VARIANT 52..56: note = This region may encompass "SGGQQ," "GAGQQ," "GQGPY," "AGQQ" or "SQ," wherein some positions may be absent
  VARIANT 60..64: note = This region may encompass "SGGQQ," "GAGQQ," "GQGPY," "AGQQ" or "SQ," wherein some positions may be absent
  VARIANT 1..64: note = This region may encompass 4-8 "GPG-X1" repeating units, wherein X1 is "SGGQQ," "GAGQQ," "GQGPY," "AGQQ" or "SQ," and some positions may be absent
GPGXXXXXGPGXXXXXGPGXXXXXGPGXXXXXGPGXXXXXGPGXXXXXGPGXXXXXGPGXXXXX

SEQ ID NO: 148 - protein, 20 aa, synthetic construct
  VARIANT 1..20: note = This sequence may encompass 6-20 residues
AAAAAAAAAAAAAAAAAAAA

SEQ ID NO: 149 - protein, 1800 aa, synthetic construct
  VARIANT 7..11: note = This region may encompass "SGGQQ," "GAGQQ," "GQGPY," "AGQQ" or "SQ," wherein some positions may be absent
  VARIANT 15..19: note = This region may encompass "SGGQQ," "GAGQQ," "GQGPY," "AGQQ" or "SQ," wherein some positions may be absent
  VARIANT 23..27: note = This region may encompass "SGGQQ," "GAGQQ," "GQGPY," "AGQQ" or "SQ," wherein some positions may be absent
  VARIANT 31..35: note = This region may encompass "SGGQQ," "GAGQQ," "GQGPY," "AGQQ" or "SQ," wherein some positions may be absent
  VARIANT 39..43: note = This region may encompass "SGGQQ," "GAGQQ," "GQGPY," "AGQQ" or "SQ," wherein some positions may be absent
  VARIANT 47..51: note = This region may encompass "SGGQQ," "GAGQQ," "GQGPY," "AGQQ" or "SQ," wherein some positions may be absent
  VARIANT 55..59: note = This region may encompass "SGGQQ," "GAGQQ," "GQGPY," "AGQQ" or "SQ," wherein some positions may be absent
  VARIANT 63..67: note = This region may encompass "SGGQQ," "GAGQQ," "GQGPY," "AGQQ" or "SQ," wherein some positions may be absent
  VARIANT 4..67: note = This region may encompass 4-8 repeating "GPG-X1" repeating units, wherein X1 is "SGGQQ," "GAGQQ," "GQGPY," "AGQQ" or "SQ," and some positions may be absent
  VARIANT 71..90: note = This region may encompass 6-20 residues
  VARIANT 97..101: note = This region may encompass "SGGQQ," "GAGQQ," "GQGPY," "AGQQ" or "SQ," wherein some positions may be absent
  VARIANT 105..109: note = This region may encompass "SGGQQ," "GAGQQ," "GQGPY," "AGQQ" or "SQ," wherein some positions may be absent
  VARIANT 113..117: note = This region may encompass "SGGQQ," "GAGQQ," "GQGPY," "AGQQ" or "SQ," wherein some positions may be absent
  VARIANT 121..125: note = This region may encompass "SGGQQ," "GAGQQ," "GQGPY," "AGQQ" or "SQ," wherein some positions may be absent
  VARIANT 129..133: note = This region may encompass "SGGQQ," "GAGQQ," "GQGPY," "AGQQ" or "SQ," wherein some positions may be absent
  VARIANT 137..141: note = This region may encompass "SGGQQ," "GAGQQ," "GQGPY," "AGQQ" or "SQ," wherein some positions may be absent
  VARIANT 145..149: note = This region may encompass "SGGQQ," "GAGQQ," "GQGPY," "AGQQ" or "SQ," wherein some positions may be absent
  VARIANT 153..157: note = This region may encompass "SGGQQ," "GAGQQ," "GQGPY," "AGQQ" or "SQ," wherein some positions may be absent
  VARIANT 94..157: note = This region may encompass 4-8 repeating "GPG-X1" repeating units, wherein X1 is "SGGQQ," "GAGQQ," "GQGPY," "AGQQ" or "SQ," and some positions may be absent
  VARIANT 161..180: note = This region may encompass 6-20 residues
  VARIANT 187..191: note = This region may encompass "SGGQQ," "GAGQQ," "GQGPY," "AGQQ" or "SQ," wherein some positions may be absent
  VARIANT 195..199: note = This region may encompass "SGGQQ," "GAGQQ," "GQGPY," "AGQQ" or "SQ," wherein some positions may be absent
  VARIANT 203..207: note = This region may encompass "SGGQQ," "GAGQQ," "GQGPY," "AGQQ" or "SQ," wherein some positions may be absent
  VARIANT 211..215: note = This region may encompass "SGGQQ," "GAGQQ," "GQGPY," "AGQQ" or "SQ," wherein some positions may be absent
  VARIANT 219..223: note = This region may encompass "SGGQQ," "GAGQQ," "GQGPY," "AGQQ" or "SQ," wherein some positions may be absent
  VARIANT 227..231: note = This region may encompass "SGGQQ," "GAGQQ," "GQGPY," "AGQQ" or "SQ," wherein some positions may be absent
  VARIANT 235..239: note = This region may encompass "SGGQQ," "GAGQQ," "GQGPY," "AGQQ" or "SQ," wherein some positions may be absent
  VARIANT 243..247: note = This region may encompass "SGGQQ," "GAGQQ," "GQGPY," "AGQQ" or "SQ," wherein some positions may be absent
  VARIANT 184..247: note = This region may encompass 4-8 repeating "GPG-X1" repeating units, wherein X1 is "SGGQQ," "GAGQQ," "GQGPY," "AGQQ" or "SQ," and some positions may be absent
  VARIANT 251..270: note = This region may encompass 6-20 residues
  VARIANT 277..281: note = This region may encompass "SGGQQ," "GAGQQ," "GQGPY," "AGQQ" or "SQ," wherein some positions may be absent
  VARIANT 285..289: note = This region may encompass "SGGQQ," "GAGQQ," "GQGPY," "AGQQ" or "SQ," wherein some positions may be absent
  VARIANT 293..297: note = This region may encompass "SGGQQ," "GAGQQ," "GQGPY," "AGQQ" or "SQ," wherein some positions may be absent
  VARIANT 301..305: note = This region may encompass "SGGQQ," "GAGQQ," "GQGPY," "AGQQ" or "SQ," wherein some positions may be absent
  VARIANT 309..313: note = This region may encompass "SGGQQ," "GAGQQ," "GQGPY," "AGQQ" or "SQ," wherein some positions may be absent
  VARIANT 317..321: note = This region may encompass "SGGQQ," "GAGQQ," "GQGPY," "AGQQ" or "SQ," wherein some positions may be absent
  VARIANT 325..329: note = This region may encompass "SGGQQ," "GAGQQ," "GQGPY," "AGQQ" or "SQ," wherein some positions may be absent
  VARIANT 333..337: note = This region may encompass "SGGQQ," "GAGQQ," "GQGPY," "AGQQ" or "SQ," wherein some positions may be absent
  VARIANT 274..337: note = This region may encompass 4-8 repeating "GPG-X1" repeating units, wherein X1 is "SGGQQ," "GAGQQ," "GQGPY," "AGQQ" or "SQ," and some positions may be absent
  VARIANT 341..360: note = This region may encompass 6-20 residues
  VARIANT 367..371: note = This region may encompass "SGGQQ," "GAGQQ," "GQGPY," "AGQQ" or "SQ," wherein some positions may be absent
  VARIANT 375..379: note = This region may encompass "SGGQQ," "GAGQQ," "GQGPY," "AGQQ" or "SQ," wherein some positions may be absent
  VARIANT 383..387: note = This region may encompass "SGGQQ," "GAGQQ," "GQGPY," "AGQQ" or "SQ," wherein some positions may be absent
  VARIANT 391..395: note = This region may encompass "SGGQQ," "GAGQQ," "GQGPY," "AGQQ" or "SQ," wherein some positions may be absent
  VARIANT 399..403: note = This region may encompass "SGGQQ," "GAGQQ," "GQGPY," "AGQQ" or "SQ," wherein some positions may be absent
  VARIANT 407..411: note = This region may encompass "SGGQQ," "GAGQQ," "GQGPY," "AGQQ" or "SQ," wherein some positions may be absent
  VARIANT 415..419: note = This region may encompass "SGGQQ," "GAGQQ," "GQGPY," "AGQQ" or "SQ," wherein some positions may be absent
  VARIANT 423..427: note = This region may encompass "SGGQQ," "GAGQQ," "GQGPY," "AGQQ" or "SQ," wherein some positions may be absent
  VARIANT 364..427: note = This region may encompass 4-8 repeating "GPG-X1" repeating units, wherein X1 is "SGGQQ," "GAGQQ," "GQGPY," "AGQQ" or "SQ," and some positions may be absent
  VARIANT 431..450: note = This region may encompass 6-20 residues
  VARIANT 457..461: note = This region may encompass "SGGQQ," "GAGQQ," "GQGPY," "AGQQ" or "SQ," wherein some positions may be absent
  VARIANT 465..469: note = This region may encompass "SGGQQ," "GAGQQ," "GQGPY," "AGQQ" or "SQ," wherein some positions may be absent
  VARIANT 473..477: note = This region may encompass "SGGQQ," "GAGQQ," "GQGPY," "AGQQ" or "SQ," wherein some positions may be absent
  VARIANT 481..485: note = This region may encompass "SGGQQ," "GAGQQ," "GQGPY," "AGQQ" or "SQ," wherein some positions may be absent
  VARIANT 489..493: note = This region may encompass "SGGQQ," "GAGQQ," "GQGPY," "AGQQ" or "SQ," wherein some positions may be absent
  VARIANT 497..501: note = This region may encompass "SGGQQ," "GAGQQ," "GQGPY," "AGQQ" or "SQ," wherein some positions may be absent
  VARIANT 505..509: note = This region may encompass "SGGQQ," "GAGQQ," "GQGPY," "AGQQ" or "SQ," wherein some positions may be absent
  VARIANT 513..517: note = This region may encompass "SGGQQ," "GAGQQ," "GQGPY," "AGQQ" or "SQ," wherein some positions may be absent
  VARIANT 454..517: note = This region may encompass 4-8 repeating "GPG-X1" repeating units, wherein X1 is "SGGQQ," "GAGQQ," "GQGPY," "AGQQ" or "SQ," and some positions may be absent
  VARIANT 521..540: note = This region may encompass 6-20 residues
  VARIANT 547..551: note = This region may encompass "SGGQQ," "GAGQQ," "GQGPY," "AGQQ" or "SQ," wherein some positions may be absent
  VARIANT 555..559: note = This region may encompass "SGGQQ," "GAGQQ," "GQGPY," "AGQQ" or "SQ," wherein some positions may be absent
  VARIANT 563..567: note = This region may encompass "SGGQQ," "GAGQQ," "GQGPY," "AGQQ" or "SQ," wherein some positions may be absent
  VARIANT 571..575: note = This region may encompass "SGGQQ," "GAGQQ," "GQGPY," "AGQQ" or "SQ," wherein some positions may be absent
  VARIANT 579..583: note = This region may encompass "SGGQQ," "GAGQQ," "GQGPY," "AGQQ" or "SQ," wherein some positions may be absent
  VARIANT 587..591: note = This region may encompass "SGGQQ," "GAGQQ," "GQGPY," "AGQQ" or "SQ," wherein some positions may be absent
  VARIANT 595..599: note = This region may encompass "SGGQQ," "GAGQQ," "GQGPY," "AGQQ" or "SQ," wherein some positions may be absent
  VARIANT 603..607: note = This region may encompass "SGGQQ," "GAGQQ," "GQGPY," "AGQQ" or "SQ," wherein some positions may be absent
  VARIANT 544..607: note = This region may encompass 4-8 repeating "GPG-X1" repeating units, wherein X1 is "SGGQQ," "GAGQQ," "GQGPY," "AGQQ" or "SQ," and some positions may be absent
  VARIANT 611..630: note = This region may encompass 6-20 residues
  VARIANT 637..641: note = This region may encompass "SGGQQ," "GAGQQ," "GQGPY," "AGQQ" or "SQ," wherein some positions may be absent
  VARIANT 645..649: note = This region may encompass "SGGQQ," "GAGQQ," "GQGPY," "AGQQ" or "SQ," wherein some positions may be absent
  VARIANT 653..657: note = This region may encompass "SGGQQ," "GAGQQ," "GQGPY," "AGQQ" or "SQ," wherein some positions may be absent
  VARIANT 661..665: note = This region may encompass "SGGQQ," "GAGQQ," "GQGPY," "AGQQ" or "SQ," wherein some positions may be absent
  VARIANT 669..673: note = This region may encompass "SGGQQ," "GAGQQ," "GQGPY," "AGQQ" or "SQ," wherein some positions may be absent
  VARIANT 677..681: note = This region may encompass "SGGQQ," "GAGQQ," "GQGPY," "AGQQ" or "SQ," wherein some positions may be absent
  VARIANT 685..689: note = This region may encompass "SGGQQ," "GAGQQ," "GQGPY," "AGQQ" or "SQ," wherein some positions may be absent
  VARIANT 693..697: note = This region may encompass "SGGQQ," "GAGQQ," "GQGPY," "AGQQ" or "SQ," wherein some positions may be absent
  VARIANT 634..697: note = This region may encompass 4-8 repeating "GPG-X1" repeating units, wherein X1 is "SGGQQ," "GAGQQ," "GQGPY," "AGQQ" or "SQ," and some positions may be absent
  VARIANT 701..720: note = This region may encompass 6-20 residues
  VARIANT 727..731: note = This region may encompass "SGGQQ," "GAGQQ," "GQGPY," "AGQQ" or "SQ," wherein some positions may be absent
  VARIANT 735..739: note = This region may encompass "SGGQQ," "GAGQQ," "GQGPY," "AGQQ" or "SQ," wherein some positions may be absent
  VARIANT 743..747: note = This region may encompass "SGGQQ," "GAGQQ," "GQGPY," "AGQQ" or "SQ," wherein some positions may be absent
  VARIANT 751..755: note = This region may encompass "SGGQQ," "GAGQQ," "GQGPY," "AGQQ" or "SQ," wherein some positions may be absent
  VARIANT 759..763: note = This region may encompass "SGGQQ," "GAGQQ," "GQGPY," "AGQQ" or "SQ," wherein some positions may be absent
  VARIANT 767..771: note = This region may encompass "SGGQQ," "GAGQQ," "GQGPY," "AGQQ" or "SQ," wherein some positions may be absent
  VARIANT 775..779: note = This region may encompass "SGGQQ," "GAGQQ," "GQGPY," "AGQQ" or "SQ," wherein some positions may be absent
  VARIANT 783..787: note = This region may encompass "SGGQQ," "GAGQQ," "GQGPY," "AGQQ" or "SQ," wherein some positions may be absent
  VARIANT 724..787: note = This region may encompass 4-8 repeating "GPG-X1" repeating units, wherein X1 is "SGGQQ," "GAGQQ," "GQGPY," "AGQQ" or "SQ," and some positions may be absent
  VARIANT 791..810: note = This region may encompass 6-20 residues
  VARIANT 817..821: note = This region may encompass "SGGQQ," "GAGQQ," "GQGPY," "AGQQ" or "SQ," wherein some positions may be absent
  VARIANT 825..829: note = This region may encompass "SGGQQ," "GAGQQ," "GQGPY," "AGQQ" or "SQ," wherein some positions may be absent
  VARIANT 833..837: note = This region may encompass "SGGQQ," "GAGQQ," "GQGPY," "AGQQ" or "SQ," wherein some positions may be absent
  VARIANT 841..845: note = This region may encompass "SGGQQ," "GAGQQ," "GQGPY," "AGQQ" or "SQ," wherein some positions may be absent
  VARIANT 849..853: note = This region may encompass "SGGQQ," "GAGQQ," "GQGPY," "AGQQ" or "SQ," wherein some positions may be absent
  VARIANT 857..861: note = This region may encompass "SGGQQ," "GAGQQ," "GQGPY," "AGQQ" or "SQ," wherein some positions may be absent
  VARIANT 865..869: note = This region may encompass "SGGQQ," "GAGQQ," "GQGPY," "AGQQ" or "SQ," wherein some positions may be absent
  VARIANT 873..877: note = This region may encompass "SGGQQ," "GAGQQ," "GQGPY," "AGQQ" or "SQ," wherein some positions may be absent
  VARIANT 814..877: note = This region may encompass 4-8 repeating "GPG-X1" repeating units, wherein X1 is "SGGQQ," "GAGQQ," "GQGPY," "AGQQ" or "SQ," and some positions may be absent
  VARIANT 881..900: note = This region may encompass 6-20 residues
  VARIANT 907..911: note = This region may encompass "SGGQQ," "GAGQQ," "GQGPY," "AGQQ" or "SQ," wherein some positions may be absent
  VARIANT 915..919: note = This region may encompass "SGGQQ," "GAGQQ," "GQGPY," "AGQQ" or "SQ," wherein some positions may be absent
  VARIANT 923..927: note = This region may encompass "SGGQQ," "GAGQQ," "GQGPY," "AGQQ" or "SQ," wherein some positions may be absent
  VARIANT 931..935: note = This region may encompass "SGGQQ," "GAGQQ," "GQGPY," "AGQQ" or "SQ," wherein some positions may be absent
  VARIANT 939..943: note = This region may encompass "SGGQQ," "GAGQQ," "GQGPY," "AGQQ" or "SQ," wherein some positions may be absent
  VARIANT 947..951: note = This region may encompass "SGGQQ," "GAGQQ," "GQGPY," "AGQQ" or "SQ," wherein some positions may be absent
  VARIANT 955..959: note = This region may encompass "SGGQQ," "GAGQQ," "GQGPY," "AGQQ" or "SQ," wherein some positions may be absent
  VARIANT 963..967: note = This region may encompass "SGGQQ," "GAGQQ," "GQGPY," "AGQQ" or "SQ," wherein some positions may be absent
  VARIANT 904..967: note = This region may encompass 4-8 repeating "GPG-X1" repeating units, wherein X1 is "SGGQQ," "GAGQQ," "GQGPY," "AGQQ" or "SQ," and some positions may be absent
  VARIANT 971..990: note = This region may encompass 6-20 residues
  VARIANT 997..1001: note = This region may encompass "SGGQQ," "GAGQQ," "GQGPY," "AGQQ" or "SQ," wherein some positions may be absent
  VARIANT 1005..1009: note = This region may encompass "SGGQQ," "GAGQQ," "GQGPY," "AGQQ" or "SQ," wherein some positions may be absent
  VARIANT 1013..1017: note = This region may encompass "SGGQQ," "GAGQQ," "GQGPY," "AGQQ" or "SQ," wherein some positions may be absent
  VARIANT 1021..1025: note = This region may encompass "SGGQQ," "GAGQQ," "GQGPY," "AGQQ" or "SQ," wherein some positions may be absent
  VARIANT 1029..1033: note = This region may encompass "SGGQQ," "GAGQQ," "GQGPY," "AGQQ" or "SQ," wherein some positions may be absent
  VARIANT 1037..1041: note = This region may encompass "SGGQQ," "GAGQQ," "GQGPY," "AGQQ" or "SQ," wherein some positions may be absent
  VARIANT 1045..1049: note = This region may encompass "SGGQQ," "GAGQQ," "GQGPY," "AGQQ" or "SQ," wherein some positions may be absent
  VARIANT 1053..1057: note = This region may encompass "SGGQQ," "GAGQQ," "GQGPY," "AGQQ" or "SQ," wherein some positions may be absent
  VARIANT 994..1057: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1061..1080: note = This region may encompass 6-20 residues
  VARIANT 1087..1091: note = This region may encompass "SGGQQ," "GAGQQ," "GQGPY," "AGQQ" or "SQ," wherein some positions may be absent
  VARIANT 1095..1099: note = This region may encompass "SGGQQ," "GAGQQ," "GQGPY," "AGQQ" or "SQ," wherein some positions may be absent
  VARIANT 1103..1107: note = This region may encompass "SGGQQ," "GAGQQ," "GQGPY," "AGQQ" or "SQ," wherein some positions may be absent
  VARIANT 1111..1115: note = This region may encompass "SGGQQ," "GAGQQ," "GQGPY," "AGQQ" or "SQ," wherein some positions may be absent
  VARIANT 1119..1123: note = This region may encompass "SGGQQ," "GAGQQ," "GQGPY," "AGQQ" or "SQ," wherein some positions may be absent
  VARIANT 1127..1131: note = This region may encompass "SGGQQ," "GAGQQ," "GQGPY," "AGQQ" or "SQ," wherein some positions may be absent
  VARIANT 1135..1139: note = This region may encompass "SGGQQ," "GAGQQ," "GQGPY," "AGQQ" or "SQ," wherein some positions may be absent
  VARIANT 1143..1147: note = This region may encompass "SGGQQ," "GAGQQ," "GQGPY," "AGQQ" or "SQ," wherein some positions may be absent
  VARIANT 1084..1147: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1151..1170: note = This region may encompass 6-20 residues
  VARIANT 1177..1181: note = This region may encompass "SGGQQ," "GAGQQ," "GQGPY," "AGQQ" or "SQ," wherein some positions may be absent
  VARIANT 1185..1189: note = This region may encompass "SGGQQ," "GAGQQ," "GQGPY," "AGQQ" or "SQ," wherein some positions may be absent
  VARIANT 1193..1197: note = This region may encompass "SGGQQ," "GAGQQ," "GQGPY," "AGQQ" or "SQ," wherein some positions may be absent
  VARIANT 1201..1205: note = This region may encompass "SGGQQ," "GAGQQ," "GQGPY," "AGQQ" or "SQ," wherein some positions may be absent
  VARIANT 1209..1213: note = This region may encompass "SGGQQ," "GAGQQ," "GQGPY," "AGQQ" or "SQ," wherein some positions may be absent
  VARIANT 1217..1221: note = This region may encompass "SGGQQ," "GAGQQ," "GQGPY," "AGQQ" or "SQ," wherein some positions may be absent
  VARIANT 1225..1229: note = This region may encompass "SGGQQ," "GAGQQ," "GQGPY," "AGQQ" or "SQ," wherein some positions may be absent
  VARIANT 1233..1237: note = This region may encompass "SGGQQ," "GAGQQ," "GQGPY," "AGQQ" or "SQ," wherein some positions may be absent
  VARIANT 1174..1237: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1241..1260: note = This region may encompass 6-20 residues
  VARIANT 1267..1271: note = This region may encompass "SGGQQ," "GAGQQ," "GQGPY," "AGQQ" or "SQ," wherein some positions may be absent
  VARIANT 1275..1279: note = This region may encompass "SGGQQ," "GAGQQ," "GQGPY," "AGQQ" or "SQ," wherein some positions may be absent
  VARIANT 1283..1287: note = This region may encompass "SGGQQ," "GAGQQ," "GQGPY," "AGQQ" or "SQ," wherein some positions may be absent
  VARIANT 1291..1295: note = This region may encompass "SGGQQ," "GAGQQ," "GQGPY," "AGQQ" or "SQ," wherein some positions may be absent
  VARIANT 1299..1303: note = This region may encompass "SGGQQ," "GAGQQ," "GQGPY," "AGQQ" or "SQ," wherein some positions may be absent
  VARIANT 1307..1311: note = This region may encompass "SGGQQ," "GAGQQ," "GQGPY," "AGQQ" or "SQ," wherein some positions may be absent
  VARIANT 1315..1319: note = This region may encompass "SGGQQ," "GAGQQ," "GQGPY," "AGQQ" or "SQ," wherein some positions may be absent
  VARIANT 1323..1327: note = This region may encompass "SGGQQ," "GAGQQ," "GQGPY," "AGQQ" or "SQ," wherein some positions may be absent
  VARIANT 1264..1327: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1331..1350: note = This region may encompass 6-20 residues
  VARIANT 1357..1361: note = This region may encompass "SGGQQ," "GAGQQ," "GQGPY," "AGQQ" or "SQ," wherein some positions may be absent
  VARIANT 1365..1369: note = This region may encompass "SGGQQ," "GAGQQ," "GQGPY," "AGQQ" or "SQ," wherein some positions may be absent
  VARIANT 1373..1377: note = This region may encompass "SGGQQ," "GAGQQ," "GQGPY," "AGQQ" or "SQ," wherein some positions may be absent
  VARIANT 1381..1385: note = This region may encompass "SGGQQ," "GAGQQ," "GQGPY," "AGQQ" or "SQ," wherein some positions may be absent
  VARIANT 1389..1393: note = This region may encompass "SGGQQ," "GAGQQ," "GQGPY," "AGQQ" or "SQ," wherein some positions may be absent
  VARIANT 1397..1401: note = This region may encompass "SGGQQ," "GAGQQ," "GQGPY," "AGQQ" or "SQ," wherein some positions may be absent
  VARIANT 1405..1409: note = This region may encompass "SGGQQ," "GAGQQ," "GQGPY," "AGQQ" or "SQ," wherein some positions may be absent
  VARIANT 1413..1417: note = This region may encompass "SGGQQ," "GAGQQ," "GQGPY," "AGQQ" or "SQ," wherein some positions may be absent
  VARIANT 1354..1417: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1421..1440: note = This region may encompass 6-20 residues
  VARIANT 1447..1451: note = This region may encompass "SGGQQ," "GAGQQ," "GQGPY," "AGQQ" or "SQ," wherein some positions may be absent
  VARIANT 1455..1459: note = This region may encompass "SGGQQ," "GAGQQ," "GQGPY," "AGQQ" or "SQ," wherein some positions may be absent
  VARIANT 1463..1467: note = This region may encompass "SGGQQ," "GAGQQ," "GQGPY," "AGQQ" or "SQ," wherein some positions may be absent
  VARIANT 1471..1475: note = This region may encompass "SGGQQ," "GAGQQ," "GQGPY," "AGQQ" or "SQ," wherein some positions may be absent
  VARIANT 1479..1483: note = This region may encompass "SGGQQ," "GAGQQ," "GQGPY," "AGQQ" or "SQ," wherein some positions may be absent
  VARIANT 1487..1491: note = This region may encompass "SGGQQ," "GAGQQ," "GQGPY," "AGQQ" or "SQ," wherein some positions may be absent
  VARIANT 1495..1499: note = This region may encompass "SGGQQ," "GAGQQ," "GQGPY," "AGQQ" or "SQ," wherein some positions may be absent
  VARIANT 1503..1507: note = This region may encompass "SGGQQ," "GAGQQ," "GQGPY," "AGQQ" or "SQ," wherein some positions may be absent
  VARIANT 1444..1507: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1511..1530: note = This region may encompass 6-20 residues
  VARIANT 1537..1541: note = This region may encompass "SGGQQ," "GAGQQ," "GQGPY," "AGQQ" or "SQ," wherein some positions may be absent
  VARIANT 1545..1549: note = This region may encompass "SGGQQ," "GAGQQ," "GQGPY," "AGQQ" or "SQ," wherein some positions may be absent
  VARIANT 1553..1557: note = This region may encompass "SGGQQ," "GAGQQ," "GQGPY," "AGQQ" or "SQ," wherein some positions may be absent
  VARIANT 1561..1565: note = This region may encompass "SGGQQ," "GAGQQ," "GQGPY," "AGQQ" or "SQ," wherein some positions may be absent
  VARIANT 1569..1573: note = This region may encompass "SGGQQ," "GAGQQ," "GQGPY," "AGQQ" or "SQ," wherein some positions may be absent
  VARIANT 1577..1581: note = This region may encompass "SGGQQ," "GAGQQ," "GQGPY," "AGQQ" or "SQ," wherein some positions may be absent
  VARIANT 1585..1589: note = This region may encompass "SGGQQ," "GAGQQ," "GQGPY," "AGQQ" or "SQ," wherein some positions may be absent
  VARIANT 1593..1597: note = This region may encompass "SGGQQ," "GAGQQ," "GQGPY," "AGQQ" or "SQ," wherein some positions may be absent
  VARIANT 1534..1597: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1601..1620: note = This region may encompass 6-20 residues
  VARIANT 1627..1631: note = This region may encompass "SGGQQ," "GAGQQ," "GQGPY," "AGQQ" or "SQ," wherein some positions may be absent
  VARIANT 1635..1639: note = This region may encompass "SGGQQ," "GAGQQ," "GQGPY," "AGQQ" or "SQ," wherein some positions may be absent
  VARIANT 1643..1647: note = This region may encompass "SGGQQ," "GAGQQ," "GQGPY," "AGQQ" or "SQ," wherein some positions may be absent
  VARIANT 1651..1655: note = This region may encompass "SGGQQ," "GAGQQ," "GQGPY," "AGQQ" or "SQ," wherein some positions may be absent
  VARIANT 1659..1663: note = This region may encompass "SGGQQ," "GAGQQ," "GQGPY," "AGQQ" or "SQ," wherein some positions may be absent
  VARIANT 1667..1671: note = This region may encompass "SGGQQ," "GAGQQ," "GQGPY," "AGQQ" or "SQ," wherein some positions may be absent
  VARIANT 1675..1679: note = This region may encompass "SGGQQ," "GAGQQ," "GQGPY," "AGQQ" or "SQ," wherein some positions may be absent
  VARIANT 1683..1687: note = This region may encompass "SGGQQ," "GAGQQ," "GQGPY," "AGQQ" or "SQ," wherein some positions may be absent
  VARIANT 1624..1687: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1691..1710: note = This region may encompass 6-20 residues
  VARIANT 1717..1721: note = This region may encompass "SGGQQ," "GAGQQ," "GQGPY," "AGQQ" or "SQ," wherein some positions may be absent
  VARIANT 1725..1729: note = This region may encompass "SGGQQ," "GAGQQ," "GQGPY," "AGQQ" or "SQ," wherein some positions may be absent
  VARIANT 1733..1737: note = This region may encompass "SGGQQ," "GAGQQ," "GQGPY," "AGQQ" or "SQ," wherein some positions may be absent
  VARIANT 1741..1745: note = This region may encompass "SGGQQ," "GAGQQ," "GQGPY," "AGQQ" or "SQ," wherein some positions may be absent
  VARIANT 1749..1753: note = This region may encompass "SGGQQ," "GAGQQ," "GQGPY," "AGQQ" or "SQ," wherein some positions may be absent
  VARIANT 1757..1761: note = This region may encompass "SGGQQ," "GAGQQ," "GQGPY," "AGQQ" or "SQ," wherein some positions may be absent
  VARIANT 1765..1769: note = This region may encompass "SGGQQ," "GAGQQ," "GQGPY," "AGQQ" or "SQ," wherein some positions may be absent
  VARIANT 1773..1777: note = This region may encompass "SGGQQ," "GAGQQ," "GQGPY," "AGQQ" or "SQ," wherein some positions may be absent
  VARIANT 1714..1777: note = This region may encompass 4-8 repeating "GPG-X1" repeating units, wherein X1 is "SGGQQ," "GAGQQ," "GQGPY," "AGQQ" or "SQ," and some positions may be absent
  VARIANT 1781..1800: note = This region may encompass 6-20 residues
  VARIANT 1..1800: note = This sequence may encompass 2-20 "GGY-[GPG-X1]n1-GPS-(A)n2" repeating units, wherein X1 is "SGGQQ," "GAGQQ," "GQGPY," "AGQQ" or "SQ," n1 is 4-8 and n2 is 6-20 and some positions may be absent
GGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAA

SEQ ID NO: 150 - protein, 5 aa, synthetic construct
SGGQQ

SEQ ID NO: 151 - protein, 5 aa, synthetic construct
GAGQQ

SEQ ID NO: 152 - protein, 5 aa, synthetic construct
GQGPY

SEQ ID NO: 153 - protein, 4 aa, synthetic construct
AGQQ

SEQ ID NO: 154 - protein, 8 aa, synthetic construct
  VARIANT 1..8: note = This sequence may encompass 6-8 residues
HHHHHHHH